(19) United States
(12) Patent Application Publication
(10) Pub. No.: US 2016/0025704 A1
(43) Pub. Date: Jan. 28, 2016

(54) METHODS FOR IDENTIFYING NEUROPROTECTIVE PKC ACTIVATORS

(71) Applicant: Daniel L. ALKON, (US)

(72) Inventor: Daniel L. ALKON, Chevy Chase, MD (US)

(21) Appl. No.: 14/774,809

(22) PCT Filed: Mar. 15, 2014
(86) PCT No.: PCT/US2014/030055

§ 371 Date: Sep. 11, 2015

Related U.S. Application Data
(60) Provisional application No. 61/791,758, filed on Mar. 15, 2013.

(51) Int. Cl. G01N 33/50; G01N 33/573; C12Q 1/68

(52) U.S. Cl. CPC G01N 33/5008; C12Q 1/6883; G01N 33/573; G01N 2500/10; G01N 2500/20; G01N 2500/04; G01N 2333/912; C12Q 2600/158; C12Q 2600/136

(57) ABSTRACT

The present disclosure is directed to methods of identifying neuroprotective PKC activators comprising analyzing candidate PKC activators to determine if they are non-tumorigenic, non-toxic, accessible to the brain, have α and ε specificity, result in minimal down regulation of the ε isozyme, are synapatogenic, and are anti-apoptotic. The methods disclosed herein may further comprise analyzing candidate PKC activators to determine whether they are neuroprotective against ASPD, protect against in vivo neurodegeneration, enhance learning and memory in normal animal models, induce downstream synaptogenic biochemical events, activate A-β degrading enzymes, inhibit GSK-3β, and/or activate alpha-secretase.

This application claims the benefit of priority under 35 U.S.C. §119 to U.S. Provisional Application No. 61/791,758, filed on Mar. 15, 2013, the content of which is incorporated by reference in its entirety.

PKC is one of the largest gene families of non-receptor serine-threonine protein kinases. Since the discovery of PKC in the early eighties and its identification as a major receptor for phorbol esters, a multitude of physiological signaling mechanisms have been ascribed to this enzyme. Kikkawa et al., J. Biol. Chem. (1982), vol. 257, pp. 13341-13348; Ashendel et al., Cancer Res. (1983), vol. 43: 4333-4337. The interest in PKC stems from its unique ability to be activated in vitro by calcium and diacylglycerol (and phorbol ester mimetics), an effector whose formation is coupled to phospholipid turnover by the action of growth and differentiation factors. Activation of PKC involves binding of 1,2-diacylglycerol (DAG) and/or 1,2-diacyl-sn-glycero-3-phospho-L-serine (phosphatidyl-L-serine, PS) at different binding sites. An alternative approach to activating PKC directly is through indirect PKC activation, e.g., by activating phospholipases such as phospholipase Cy, by stimulating the Ser/Thr kinase Akt by way of phosphatidylinositol 3-kinase (PI3K), or by increasing the levels of DAG, the endogenous activator. Nelson et al., Trends in Biochem. Sci. (2009) vol. 34, pp. 136-145. Diacylglycerol kinase inhibitors, for example, may enhance the levels of the endogenous ligand diacylglycerol, thereby producing activation of PKC. Meinhardt et al., Anti-Cancer Drugs (2002), vol. 13, pp. 725-733. Phorbol esters are not suitable compounds for eventual drug development because of their tumor promotion activity. Ibarreta et al. Neuroreport (1999), vol. 10, pp. 1035-1040).

The PKC gene family consists of 11 genes, which are divided into four subgroups: (1) classical PKC α, β1, β2 (β1 and β2 are alternatively spliced forms of the same gene) and γ; (2) novel PKC δ, ε, η, and θ; (3) atypical PKC and ζ, τ/λ; and (4) PKC μ. PKC μ resembles the novel PKC isoforms but differs by having a putative transmembrane domain. Blobe et al. Cancer Metastasis Rev. (1994), vol. 13, pp. 411-431; Hug et al. Biochem. J. (1993) vol. 291, pp. 329-343; Kikkawa et al. Ann. Rev. Biochem. (1989), vol. 58, pp. 31-44. The classical PKC isoforms α, β1, β2, and γ are Ca²⁺, phospholipid, and diacylglycerol-dependent, whereas the other isoforms are activated by phospholipid, diacylglycerol but are not dependent on Ca²⁺ and no activator may be necessary. All isoforms encompass 5 variable (VI-V5) regions, and the α, β, and γ isoforms contain four (C1-C4) structural domains which are highly conserved. All isoforms except PKC α, β, and γ lack the C2 domain, the τ/λ and η isoforms also lack nine of two cysteine-rich zinc finger domains in C1 to which diacylglycerol binds. The C1 domain also contains the pseudosubstrate sequence which is highly conserved among all isoforms, and which serves an autoregulatory function by blocking the substrate-binding site to produce an inactive conformation of the enzyme. House et al., Science (1987), vol. 238, pp. 1726-1728.

Because of these structural features, diverse PKC isoforms are thought to have highly specialized roles in signal transduction in response to physiological stimuli as well as in neoplastic transformation and differentiation. Nishizuka, Cancer (1989), vol. 10, pp. 1892-1903; Glazer, pp. 171-198 in Protein Kinase C, 1.F. Kuo, ed., Oxford U. Press, 1994. For a discussion of PKC modulators see, for example, International Application No. PCT/US97/08141 (WO 97/43268) and U.S. Pat. Nos. 5,652,232; 6,080,784; 5,891,906; 5,962,498; 5,955,501; 5,891,870 and 5,962,504, each is incorporated by reference herein in its entirety.

The activation of PKC has been shown to improve learning and memory. See, e.g., Hongpaisan et al., Proc. Natl. Acad. Sci. (2007) vol. 104, pp. 19571-19578; International Application Nos. PCT/US2003/007101 (WO 2003/075850); PCT/US2003/020820 (WO 2004/004641); PCT/US2005/028522 (WO 2006/031337); PCT/US2006/029110 (WO 2007/016202); PCT/US2007/002454 (WO 2008/013573); PCT/US2008/001755 (WO 2008/100449); PCT/US2008/006158 (WO 2008/143880); PCT/US2009/051927 (WO 2010/014585); and PCT/US2011/000315; and U.S. application Ser. Nos. 12/068,732; 10/167,491 (now U.S. Pat. No. 6,825,229); Ser. Nos. 12/851,222; 11/802,723; 12/068,742; and 12/510,681; each is incorporated by reference herein in its entirety. PKC activators have been used to treat memory and learning deficits induced by stroke upon administration 24 hours or more after inducing global cerebral ischemia through two-vessel occlusion combined with a short term (˜14 minutes) systemic hypoxia. Sun et al., Proc. Natl. Acad. Sci. (2008) vol. 105, pp. 13620-13625; Sun et al., Proc. Natl. Acad. Sci. (2009) vol. 106, pp. 14676-14680.

PKC Activators

PKC activators include, for example, macrocyclic lactones, bryologs, isoprenoids, daphnane-type diterpenes, bicyclic triterpenoids, napthalenesulfonamides, 8-[2-(2-pentylcyclopropyl)methyl]-cyclopropaneoctanoic acid (DCP-LA), diacylglycerol kinase inhibitors, growth factors, growth factor activators, monounsaturated fatty acids, and polyunsaturated fatty acids.

Further for example, macrocyclic lactone include, but are not limited to, bryostatin, for example, bryostatin-1, bryostatin-2, bryostatin-3, bryostatin-4, bryostatin-5, bryostatin-6, bryostatin-7, bryostatin-8, bryostatin-9, bryostatin-10, bryostatin-11, bryostatin-12, bryostatin-13, bryostatin-14, bryostatin-15, bryostatin-16, bryostatin-17, and bryostatin-18, or a neristatin, for example, neristatin-1.

Bryologs (analogs of bryostatin) are known in the art. See e.g., Wender et al., Curr. Drug Discov. Technol. (2004), vol. 1, pp. 1-11; Wender et al. Proc. Natl. Acad. Sci. (1998), vol. 95, pp. 6624-6629; Wender et al., J. Am. Chem. Soc. (2002), vol. 124, pp. 13648-13649; Wender et al., Org Lett. (2006), vol. 8, pp. 5299-5302, all incorporated by reference herein in their entireties. Bryologs are also described, for example, in U.S. Pat. Nos. 6,624,189 and 7,256,286. Non-limiting examples of bryologs include A-ring and B-ring bryologs.

Isoprenoids are PKC activators also suitable for the present disclosure, such as farnesyl thiotriazole as described in Gilbert et al., Biochemistry (1995), vol. 34, pp. 3916-3920; incorporated by reference herein in its entirety. Another example is octylindolactam V, a non-phorbol protein kinase C activator related to teleocidin, such as described in Fujiki et al. Adv. Cancer Res. (1987), vol. 49 pp. 223-264; Collins et al. Biochem. Biophys. Res. Commun. (1982), vol. 104, pp. 1159-4166, incorporated by reference herein in its entirety. Non-limiting examples of diterpenes include gnidimacrin and ingenol, and examples of triterpenoids include iripallidal. Napthalenesulfonamides, including N-(n-heptyl)-5-chloro-1-naphthalenesulfonamide (SC-10) and N-(6-phenylhexyl)-5-chloro-1-naphthalenesulfonamide, are members of another class of PKC activators, such as described by Ito et al., Biochemistry (1986), vol. 25, pp. 4179-4184, incorporated by reference herein. Diacylglycerol kinase inhibitors may also be suitable as PKC activators in the present disclosure by indirectly activating PKC, for example, 6-(2-(4-[(4-fluorophenyl)phenylmethylene]-1-piperidinyl)ethyl)-7-methyl-5H-thiazolo[3,2-a]pyrimidin-5-one (R59022) and [3-[2-[4-(bis-(4-fluorophenyl)methylene]piperidin-1-yl)ethyl]-2,3-dihydro-2-thioxo-4(1H)-quinazolinone (R59949).

A variety of growth factors, such as fibroblast growth factor 18 (FGF-18) and insulin growth factor, function through the PKC pathway, and are suitable for the methods disclosed herein. Moreover, growth factor activators include, but are not limited to 4-methyl catechol derivatives, like 4-methylcatechol acetic acid (MCBA), that stimulate the synthesis and/or activation of growth factors such as NGF and BDNF, are included herein.

Polyunsaturated fatty acids (“PUFAs”), such as arachidonic acid and 2-hydroxy-9-cis-octadecenoic acid (i.e., minerval), and PUFA derivatives, such as CPAA (cyclopropanated arachidonic acid), DCPLA (i.e., linoleic acid derivative), AA-CP4 methyl ester (i.e., arachidonic acid derivative), DHA-CP6 methyl ester (i.e., docosahexaenoic acid derivative), EPA-CP5 methyl ester (i.e., eicosapentaenoic acid derivative), and Omega-5 and Omega-7 PUFA derivatives chosen from cyclopropanated rumenic acid, cyclopropanated alphaelostearic acid, cyclopropanated catalpic acid, and cyclopropanated punicic acid, are non-limiting examples of candidate PKC activators disclosed herein.

Another class of PKC-activating fatty acids are monounsaturated fatty acid (“MUFA”) derivatives, for instance cyclopropanated oleic acid, cyclopropanated elaidic acid (shown below), and epoxylated compounds such as trans-9,10-epoxystearic acid.

In addition, cyclopropanated PUFA and MUFA fatty alcohols, cyclopropanated PUFA and MUFA fatty esters, are included as non-limiting examples of candidate PKC activator compounds.

Optimal activation of protein kinase C (“PKC”) plays a part in many molecular mechanisms that affect cognition in normal and diseased states. As such, there is a need to screen potential compounds that may be deemed neuroprotective PKC activators using various assays that test specific parameters to find suitable compounds for eventual drug development, for example, in the treatment of Alzheimer's disease. The methods of the present disclosure fulfill these needs and for example, will greatly improve the clinical treatment for Alzheimer's disease and other neurodegenerative diseases, as well as, provide for improved cognitive enhancement prophylactically.

Provided herein are methods for identifying neuroprotective PKC activators capable of protecting cells from neurodegeneration and/or for treating CNS disorders such as Alzheimer's disease. The methods disclosed herein include analyzing potential compounds to determine whether the compounds comprise certain attributes needed to protect cells from neurodegeneration and/or for treating CNS disorders such as Alzheimer's disease.

Thus, the instant disclosure is directed to methods of identifying neuroprotective PKC activators useful in the treatment of Alzheimer's disease. The disclosed methods screen PKC activator compound candidates according to the following listed criteria, referred to herein as (1) non-tumorgenicity; (2) non-toxicity; (3) brain accessibility; (4) PKC-α and PKC-ε activity; (5) minimal downregulation of PKC-ε; (6) synaptogenicity; (7) anti-apoptosis; (8) neuroprotection against ASPDs; (9) protection against in-vivo neurodegeneration; (10) enhancement of learning and memory in normal animal models; (11) induction of downstream synaptogenic biochemical events; (12) increases of activity of A-β degrading enzymes; (13) inhibition of GSK3B-phosphorylation of Tau; and (14) activation of alpha-secretase.

According to the methods disclosed herein, the candidate PKC activator is assessed using the following five criteria: brain accessibility, demonstrating PKC-α and PKC-ε activity, minimal down regulation of PKC-ε, synaptogenicity, and anti-apoptosis potential. Moreover, to be therapeutically useful, the candidate PKC activator comprises the ability to be non-tumorigenic and non-toxic. Thus, at a minimum, the candidate PKC comprises at least seven of the listed criteria in order to qualify as a neuroprotective PKC activator.

In another embodiment, the disclosed methods comprise the candidate PKC activator meeting the seven criteria defined above, but may further comprise the candidate PKC activator meeting at least one other additional criteria, for example, meeting at least eight, nine, ten, eleven, twelve, thirteen, or fourteen of the listed criteria, in order to qualify as a neuroprotective PKC activator. In at least one embodiment, the disclosed methods comprise the candidate PKC activator to be brain accessible, demonstrate PKC-α and PKC-ε activity, have minimal down regulation of PKC-ε, induce synaptogenicity, have anti-apoptosis potential, be non-tumorigenic and non-toxic, and at least one other criteria, for example, protect against ASPDs or protect agains in vivo neurodegeneration.

### BRIEF DESCRIPTION OF FIGURES

FIG. 1 shows the blood plasma levels in mice after a single intravenous injection of bryostatin.

FIG. 2 shows the difference in PKC downregulation between bryostatin levels in the brain versus bryostatin in the plasma.

FIG. 3 shows in vivo brain accessibility of PKC-ε in mice.

FIG. 4 shows the dose dependence of PKC-α and PKC-ε translocation 30 minutes after administration of bryostatin.

FIG. 5 shows the dose dependence of PKC-α and PKC-ε translocation 120 minutes after administration of bryostatin.

FIG. 6 shows the activation of various PKC isozymes by DHA-CP6, DCPLA, and DCPLA methyl ester.

FIG. 7 shows that PKC-ε activation induces synaptogenesis in primary human neurons treated with either DCPLA methyl ester or bryostatin.

FIG. 8 shows that PKC-ε activation induces neuritic branching and connections in primary human neurons treated with either DCPLA methyl ester or bryostatin.

FIG. 9 shows that PKC-ε activation induces synaptogenesis in HCN-2 cells treated with either DCPLA methyl ester or bryostatin.

FIG. 10 shows that human primary neurons treated with either DCPLA methyl ester or byrostatin prevents apoptosis.

FIG. 11 (A-C) shows that that bryostatin and DCPLA methyl ester prevents apoptotic cell death in neurons in the CA1 hippocampal area.

FIG. 12 shows a flowchart of Aβ degradation in vivo by ECE via PKC activation.

FIG. 13 (A-B) shows results of ECE activity in SH-SY5Y cells and cultured neurons by bryostatin, DCPLA, DHA-CP6, EPA-CP5, and AA-CP4.

FIG. 14 shows that primary hippocampal neuron treated with bryostatin recovers NTF mRNA expression decreased by Aβ.

FIG. 15 shows that primary hippocampal neuron treated with DCPLA recovers NTF mRNA expression decreased by Aβ.

FIG. 16 shows that primary hippocampal neuron treated with DCPLA methyl ester recovers NTF mRNA expression decreased by Aβ.

FIG. 17 shows that SH-SY5Y cells treated with bryostatin recovers membrane localization of neprilysin protein inhibited by Aβ.

FIG. 18 shows that SH-hNEP cells treated with bryostatin induces Aβ peptide degradation through neprilysin activation in vitro.

FIG. 19 (A-J) shows that bryostatin protects against the loss of postsynaptic dendritic spines and synapses in the hippocampal CA1 area in Tg2576 mice at 5 months old.

FIG. 20 (A-I) shows that DCPLA prevents synaptic loss in hippocampal CA1 area in SXFAD mice at 5 months old.

FIG. 21 (A-F) shows that bryostatin and DCPLA prevent learning and memory deficits and amyloid plaque formation in 5XFAD mice at 5 months old.

FIG. 22 (A-G) shows that bryostatin rescues learning experience and memory after cerebral ischemia is induced.

FIG. 23 (A-G) shows that bryostatin rescues learning experience and memory but not sensorimotor ability after cerebral ischemia is induced.

FIG. 24 (A-E) shows that chronic bryostatin-1 rescues pyramidal cells, neurotrophic activity, and synaptic strength in the dorsal hippocampal CA1 area from ischemia-induced damage.

FIG. 25 (A-B) shows the dose dependency of bryostatin administration in treating traumatic brain injury in rats.

FIG. 26 (A-F) shows that bryostatin restores the number of synapses in fragile X transgenic mice.

FIG. 27 (A-D) shows that bryostatin enhances mushroom spine formation in healthy rats after water maze training.

FIG. 28 (A-H) shows that bryostatin enhances memory-specific mushroom spine formation within an individual CA1 pyramidal neuron in health rats after water maze training.

FIG. 29 (A-F) shows that activated PKC induces stability in BDNF, NGF, and NT-3 transcripts.

FIG. 30 (A-H) shows that activated PKC enhances binding of HuD proteins to target NTF mRNA and increases NTF protein expression.

FIG. 31 (A-E) shows that bryostatin induces sustained activation of PKC-α dependent mRNA-stabilizing proteins ELAV or Hu and increases in dendritic spine formation and presynaptic concentration in healthy rats after water maze training.

FIG. 32 (A-B) shows that bryostatin increases neprilysin activity in brain neurons.

FIG. 33 (A-B) shows that bryostatin enhances neprilysin membrane localization and increases neprilysin activity in brain neurons.

FIG. 34 shows that bryostatin, DCPLA, and DHA-CP6 activate ECE in SH-SY5Y cells.

FIG. 35 shows that bryostatin increases phosphorylation of GSK-3β in the hippocampus of fragile X mice.

FIG. 36 (A-B) shows the variation in secretion of APP-α in human fibroblasts between bryostatin, benzolactam, and stauropsorin.

### DESCRIPTION

The methods disclosed herein are used to identify neuroprotective PKC activators capable of protecting cells from neurodegeneration and/or for treating CNS disorders such as Alzheimer's disease. Alzheimer's disease (AD), the most common form of dementia, begins with the loss of recent memory and is associated with two main pathological hallmarks in the brain: extracellular amyloid plaques and intracellular neurofibrillary tangles. These are typically associated with a significant loss of synapses. Amyloid plaques are formed by the aggregation of Aβ peptide oligomers which are generated from cleavage of the amyloid precursor protein (APP) by the β-secretase and γ-secretase pathway, while a secretase generates the non-toxic, synaptogenic soluble APP-α. Accumulated observations indicate that Protein kinase C (PKC) isozymes -α and -ε directly activate the α-secretase mediated cleavage of APP directly (Slack et al., 1993; Kinouchi et al., 1995; Jolly-Tornetta and Wolf 2000; Yeon et al., 2001. Lanni et al., 2004), and/or indirectly through phosphorylation of the extracellular signal regulated kinase (ERK½) (Devari et al., 2006, Alkon et al., 2007).

Many observations have also indicated that PKC signaling pathways regulate events in neurodegenerative pathophysiology of AD such as the endothelin converting enzyme (ECE)-mediated degradation of Aβ (Nelson et al., 2009). In vivo over-expression of PKC-ε in AD-transgenic mice reduced amyloid plaques (Choi et al., 2006).

Other studies have provided evidence that AD specific pathological abnormalities can be found in tissues other than brain which include blood, skin fibroblasts, and ocular tissues (Gurreiro et al., 2007, Ray et al., 2007). In AD skin fibroblasts, for example, defects were found of specific K⁺ channels (Etcheberrigaray et al., 1993; 1994), PKC isozymes (Govoni et al., 1993, Favit et al., 1998), Ca⁺ signaling (Ito et al., 1994), MAP kinase Erk½ phosphorylation (Zhao et al., 2002; Khan and Alkon, 2006), and PP2A (Zhao et al., 2003).

For familial AD patients, skin fibroblasts showed enhanced secretion of Aβ (Citron et al., 1994; Johnston et al., 1994) while AD-specific reduction of specific K+ channels was induced by Aβ₁₋₄₀ in normal human fibroblasts (Etcheberrigaray, et al., 1993; 1994). For example, an autopsy confirmed, internally controlled, phosphorylated Erk½ peripheral biomarker in skin fibroblasts was shown to have promising sensitivity and specificity (Khan and Alkon, 2006; 2010). Still other studies have suggested deficits of PKC in particular brain regions of AD patients (Masliah et al., 1991).

Finally, it has also been demonstrated that pharmacologic activators of PKC-α and -ε can protect two different strains of AD mice from all of the pathologic and cognitive abnormalities characteristics of AD (Hongpaisan et al., 2011). Consistent with these observations, PKC-α and -ε were found to be significantly reduced in AD transgenic mice and were restored to normal levels by treatment with pharmacologic activators of PKC-α and -ε (Hongpaisan et al., 2011).

As described above, the pathology of Alzheimer's disease is just one example of a neurological disorder that can be observed by the presence of numerous biomarkers. A benefit of drug development for treatment of neurological disorders, such as Alzheimer's disease, is to understand the effects of PKC activators on the pathology of the neurological disorder to be treated, such as, how the PKC activator affects the enhanced secretion of Aβ, and the overall effect that has on AD patients. Thus, the methods disclosed herein analyze potential neuroprotective PKC activators using various assays that test specific parameters to find suitable compounds for eventual drug development, for example, in the treatment of Alzheimer's disease.

### DEFINITIONS

As used herein, “up regulating” or “up regulation” means increasing the amount or activity of an agent, such as PKC protein or transcript, relative to a baseline state, through any mechanism including, but not limited to increased transcription, translation and/or increased stability of the transcript or protein product.

As used herein, “down regulating” or “down regulation” means decreasing the amount or activity of an agent, such as PKC protein or transcript, relative to a baseline state, through any mechanism including, but not limited to decreased transcription, translation and/or decreased stability of the transcript or protein product.

“Neurodegeneration” refers to the progressive loss of structure or function of neurons, including death of neurons.

“Synapses” are functional connections between neurons, or between neurons and other types of cells. Synapses generally connect axons to dendrites, but also connect axons to cell bodies, axons to axons, and dendrites to dendrites.

As used herein, “synaptogenesis” refers to the formation of a synapse, i.e., a process involving the formation of a neurotransmitter release site in the presynaptic neuron and a receptive field at the postsynaptic neuron. The presynaptic terminal, or synaptic bouton, is a terminal bulb at the end of an axon of the presynaptic cell that contains neurotransmitters enclosed in small membrane-bound spheres called synaptic vesicles. The dendrites of postsynaptic neurons contain neurotransmitter receptors, which are connected to a network of proteins called the postsynaptic density (PSD). Proteins in the PSD are involved in anchoring and trafficking neurotransmitter receptors and modulating the activity of these receptors. The receptors and PSDs are often found in specialized protrusions from the main dendritic shaft called dendritic spines.

The terms “therapeutically useful PKC activator” refers to a candidate PKC activator compound that results in a measurable therapeutic response. A therapeutic response may be any response that a user (e.g., a clinician) will recognize as an effective response to the therapy, including improvement of symptoms and surrogate clinical markers. Thus, a therapeutic response will generally be an amelioration or inhibition of one or more symptoms of a disease or condition e.g., AD. A measurable therapeutic response also includes a finding that a symptom or disease is prevented or has a delayed onset, or is otherwise attenuated by the therapeutic agent.

For purposes of the present disclosure, a “neurological disease” refers to any central nervous system (CNS) or peripheral nervous system (PNS) disease that is associated with the β-amyloidogenic processing of APP. This may result in neuronal or glial cell defects including, but not limited to, neuronal loss, neuronal degeneration, neuronal demyelination, gliosis (i.e., astrogliosis), or neuronal or extraneuronal accumulation of aberrant proteins or toxins (e.g., Aβ). One exemplary neurological disease is Alzheimer's Disease (AD). Another exemplary neurological disease is congophilic angiopathy (CAA), also referred to as cerebral amyloid angiopathy.

Criteria

The following subsections define criteria used to determine whether PKC compound candidate qualifies as therapeutically useful PKC activators in protecting against neurodegeneration and/or treating CNS disorders.

According to at least one embodiment of the present disclosure, the candidate PKC activator compound comprises, at a minimum, seven of the listed criteria chosen from brain accessibility, demonstrating PKC-α and PKC-ε activity, minimal down regulation of PKC-ε, synaptogenicity, anti-apoptosis potential, and be non-tumorigenic and non-toxic. In other embodiments, for example, at least eight, nine, ten, eleven, twelve, thirteen, or fourteen of the listed criteria must be met to qualify as a neuroprotective PKC activator. For example, the candidate PKC activator compound comprises the seven criteria listed above and further comprises at least one additional criteria chosen from protection against ASPD, protection against in vivo neurodegeneration, enhancement of learning and memory in a normal animal model, induction of downstream synaptogenic biochemical events, activation of A-β degrading enzymes, inhibition of GSK3β, and activation of alpha-secretase.

Non-Tumorgenicity

To be useful for therapy in CNS disorders, the candidate PKC activators are non-tumorigenic. According to the present disclosure, therefore, the candidate PKC activator is non-tumorigenic. Meaning, when the candidate PKC activator is evaluated or assessed for tumorgenicity, it results in non-tumorigenic.

Several PKC activators have been identified but some PKC activators, for example, phorbol esters, are not suitable compounds for eventual drug development because of their tumor promotion activity, (Ibarreta et al. (1999) Neuro Report 10(5&6): 1035-40). Byrostatin, unlike phorbol esters, does not promote tumor growth (proven in clinical trials) and counteracts tumor-promoting activity of phorbol esters (not proven in trials). (Phase II trial of Bryostatin 1 in Patients with Relapse Low-Grade Non-Hodgkin's Lymphoma and Chronic Lymphocytic Leukemia, Varterasian et al., Clinical Cancer Research, Vol. 6, pp. 825-28 (2000)).

Unlike tumorigenic activators, such as phorbol esters, non-tumorigenic activators do not induce macrophage-like differentiation of HL-60 cells. For example, bryostatin has been shown to block phorbol ester-induced differentiation of HL-60 cells and, if applied within 48 hours, halts further differentiation in a dose-dependent fashion. (Kraft, et al. (1986) PNAS 83(5): 1334-1338). Bryostatin has also been shown to restore the differentiation response to phorbol esters and block the induction of cellular adherence by phorbol ester. (Dell'Aquila et al. (1987) Cancer Research 47(22): 6006-6009). Structural differences may account for the differences in tumor promotion seen by various PKC activators. (Kozikowski, A P et al. (1997) J. Med. Chem. 40: 1316-1326).

PUFAs also activate PKC and are known to possess strong protection against cancer in low to moderate concentrations. (Cremonezzi, et al. (2004) Food Chem Toxicol. 42(12): 1999-2007); Silva, et al. (1995) Prostaglandins Leukot Essent Fatty Acids, 53(4): 273-277); Silva et al. (2000) Exp Toxicol Pathol. 52(1): 11-6).

One test for demonstrating non-tumorigenicity is the AMES test. The AMES test is a rapid screening of the mutagenic potential of chemical compounds. A positive test indicates that the chemical compound is mutagenic and therefore may act as a carcinogen, since cancer is often linked to mutation. Between 50% and 70% of all known carcinogens test positive in the AMES test.

Accordingly, in at least one embodiment, a candidate PKC activator compound that results in, for example, a statistically significant negative AMES test result indicates that the PKC activator can continue with the analysis of the remaining criteria in order to make a determination whether the compound is therapeutically useful in the treatment of CNS disorders. Contrariwise, if a candidate PKC activator compound results in a positive AMES test result, that candidate is not considered therapeutically useful for the methods disclosed herein.

Non-Toxicity

To be useful for therapy in CNS disorders, the potential PKC activator compounds are non-toxic. Therefore, according to the present disclosure, the candidate PKC activator is non-toxic.

Non-toxicity can be measured by administering a dose of the PKC activator and comparing changes in levels of particular biomarkers to control samples. For example, changes in internal levels of biomarkers such as proteins, lymphocytes, minerals, triglycerides, etc., may indicate toxicity and thus, is not an appropriate therapeutic option for treating CNS disorders.

Accordingly, in at least one embodiment, a PKC activator that results in, for example, a statistically significant difference in normal cellular levels of biomarkers after an effective dose of a candidate PKC activator compound is administered, indicates that the candidate PKC activator is toxic, and therefore not therapeutically useful for treating CNS disorders.

Brain Accessibility

To qualify as a useful PKC activator in protecting against neurodegeneration and in the treatment of CNS disorders, a PKC activator is access the brain. Therefore, candidate PKC activators comprise the ability to be accessible to the brain in accordance with the methods disclosed herein.

One way to measure whether a PKC activator has accessed the brain is via measurement of the PKC activator in the plasma vs. brain after administration of the PKC activator. If significant levels of the PKC activator are present in the brain after administration of the PKC activator, then that activator is brain accessible. For example, if, after a period of time after administration of the candidate PKC activator compound, the PKC activator is still present in the brain, for instance, for a time period ranging from 20 minutes to 80 minutes, such as from 30 minutes to 60 minutes, then that candidate compound is considered to have brain accessibility.

Another measure of brain accessibility is activation of PKC-ε and increased translocation. Thus, a calculated % of PKC-ε translocation in the brain as compared to control is another biomarker for identifying therapeutically useful PKC activators.

PKC-α and PKC-ε Specificity

According to the methods disclosed herein, the candidate PKC activator comprises the ability to be protective against neurodegeneration and in the treatment of CNS disorders.

It has been demonstrated that pharmacologic activators of PKC-α and -ε can protect two different strains of Alzheimer's Disease mice from all of the pathologic and cognitive abnormalities characteristics of AD (Hongpaisan et al., 2011). Consistent with these observations, PKC-α and -ε were found to be significantly reduced in AD transgenic mice and were restored to normal levels by treatment with pharmacologic activators of PKC-α and -ε (Hongpaisan et al., 2011).

Collectively, these and other previous studies have two important implications: 1) AD has systemic pathologic expression with symptomatic consequences limited to brain function, and 2) PKC isozymes particularly -α and -ε, play a critical role in regulating the major aspects of AD pathology including the loss of synapses, the generation of Aβ and amyloid plaques, and the GSK-3β-mediated hyperphosphorylation of tau in neurofibrilliary tangles.

Activation of PKC-ε by a PKC activator compound is another marker for identifying therapeutically useful PKC activators according to the methods herein. For example, measurement of PKC-ε activity levels in cells can be determined by for example, Western Blot assay, ELISA. In at least one embodiment, a PKC activator qualifies as a useful activator if it activates PKC-ε±15% and/or 30% PKC-α, PKC-δ activity, for instance activates PKC-ε ±15% PKC-α, PKC-δ activity.

Minimal Down Regulation

To qualify as a therapeutic PKC activator in the treatment of CNS disorders, a PKC activator induces minimal down regulation of PKC.

Synaptogenicity

PKC activators that induce synaptogenicity are therapeutically useful in preventing neurodegeneration and in treating CNS disorders. Thus, according to the methods disclosed herein, candidate PKC activator compounds induce synaptogenicity to be identified as therapeutically useful activators.

Memories are thought to be a result of lasting synaptic modification in the brain structures related to information processing. Synapses are considered a critical site at final targets through which memory-related events realize their functional expression, whether the events involve changed gene expression and protein translation, altered kinase activities, or modified signaling cascades. A few proteins have been implicated in associative memory including Ca2+/calmodulin II kinases, PKC, calexcitin, a 22-kDa learning-associated Ca2+ binding protein, and type II ryanodine receptors. Specifically, PKC-ε activators have been shown to enhance learning and memory as well as structurally specific synaptic changes in rat spatial maze learning (Hongpaisan and Alkon, 2007). The modulation of PKC through the administration of macrocyclic lactones is also thought to provide a mechanism to effect synaptic modification.

Activation of PKC-ε induces neurite/synaptic growth, including increasing neuritic branching and connections, increased punctate colocalization of PSD-95 and synaptophysin, and number of synapses. Those factors can be analyzed via Western Blot analysis and visualized with microscopic methods. Candidate PKC activators that show a statistically significant increase in any of the factors listed above is a positive result.

Anti-Apoptosis

PKC activators that inhibit apoptosis are therapeutically useful in preventing neurodegeneration and in treating CNS disorders. Thus, according to the methods disclosed herein, candidate PKC activator compounds inhibit apoptosis to be therapeutically useful activators.

PKC-δ and PKC-θ are often regarded as having a pro-apoptotic function because they are components of the caspase apoptosis pathway. PKC-ε, by contrast, has an opposite role: its activation promotes proliferation and cell survival, and inhibits apoptosis. See Nelson et al., Trends in Biochemical Sciences, 2009, 34(3): 136-145. Activation of PKCε may also induce synaptogenesis or prevent apoptosis following stroke or in Alzheimer's disease. For example, activation of PKC-ε protects against neurotoxic amylospheroids (ASPD)-induced apoptosis. Thus, the inhibition of apoptosis is therapeutically useful in treating CNS disorders like stroke and Alzheimer's disease.

In order to identify candidate PKC activators' potential in inhibiting apoptosis, cells can be treated with candidate PKC activator compounds and then analyzed via, for example, Western Blot analysis and visualized with microscopic methods to detect the level of apoptotic cells. Candidate PKC activators that show, for example, a statistically significant decrease in the level apoptotic cells is a positive result.

Neuroprotection Against ASPDs

PKC activators that protect against ASPDs are therapeutically useful in preventing neurodegeneration and in treating CNS disorders. Thus, according to the methods disclosed herein, candidate PKC activator compounds may also protect against ASPDs.

Amyloid plaques are one of the hallmarks of Alzheimer's disease. They are formed by the aggregation of Aβ peptide oligomers (ASPDs) which are generated from cleavage of the amyloid precursor protein (APP) by the β-secretase and γ-secretase pathway. Many observations have indicated that PKC signaling pathways regulate important events in neurodegenerative pathophysiology of AD such as the endothelin converting enzyme (ECE)-mediated degradation of Aβ (Nelson et al., 2009).

PKC signaling pathways regulate important events in neurodegenerative pathophysiology of AD such as the endothelin converting enzyme (ECE)-mediated degradation of Aβ (Nelson et al., 2009). It is possible that the different forms of toxic Aβ oligomers affect the PKC-ε levels in the cells, which is responsible for regulating the ECE, that degrades Aβ. These proteins play an important role in Aβ clearance. Thus, a reasonable hypothesis is that abnormal accumulation of Aβ due to higher β-, γ-secretase activity causes a decrease in PKC-ε that then participates in a feedback loop to cause further Aβ elevation.

An increase in ASPD levels leads to a decrease in neurotrophic factor cells (NTFs) like brain derived neurotrophic factor (BDNF), nerve growth factor (NGF), neurotrophin (NT-3), growth associated protein-43 (GAP-43), and inhibits membrane localization of neprilysin protein. PKC activators are reported to provide neuroprotection against ASPDs, possibly by activating TACE (tumor necrosis factor-α converting enzyme) and Aβ-degrading enzymes such as ECE, insulin degrading enzyme or neprilysin, or by stimulating synaptogenesis.

According to at least one embodiment in the present disclosure, therapeutically useful PKC activators will activate ECE, recover NTF mRNA expression decreased by ASPDs, and/or recover membrane localization of neprilysin protein inhibited by Aβ ologomer in neurons. A candidate PKC activator compound that results in, for example, a statistically significant increase in any of the factors listed above, is a positive result in accordance with the methods disclosed herein.

Protection Against In Vivo Neurodegeneration

A characteristic of a neuroprotective PKC activator is one that protects against in vivo neurodegeneration. Various neurological diseases or disorders can lead to neurodegeneration, such as Alzheimer's disease, stroke, traumatic brain injury, and mental retardation. Therefore, candidate PKC activator compounds may also protect against in vivo neurodegeneration in accordance with the methods disclosed herein.

One method for protecting against in vivo neurodegeneration is by protecting against neuronal loss, such as the rescue of pyramidal cells, and protecting against synaptic loss in the hippocampal CA1 area, such as the loss of postsynaptic dendritic spines, for example spinophilin; and presynaptic vesicles, for instance synaptophysin. For example, postischemic/hypoxic treatment with bryostatin-1 effectively rescued ischemia-induced deficits in synaptogenesis, neurotrophic activity, and spatial learning and memory. Sun and Alkon, Proc Natl Acad Sci USA, 2008, 105(36):13620-136255. This effect was accompanied by increases in levels of synaptic proteins spinophilin and synaptophysin, and structural changes in synaptic morphology. Hongpaisan and Alkon, Proc Natl Acad Sci USA, 2007, 104:19571-19576.

Turnover of dendritic spines has been implicated in learning and memory. In particular, long-term memory is mediated in part by the growth of new dendritic spines and the enlargement of pre-existing spines. Learning increases formation of mushroom spines, which are known to provide structural storage sites for long-term associative memory and sites for memory-specific synaptogenesis. High rates of spine turnover have also been associated with increased learning capacity, while spine persistence has been associated with memory stabilization.

Changes in dendritic spine density affect learning- and memory-induced changes in synaptic structure that increase synaptic strength. Long-term memory, for example, is mediated, in part, by the growth of new dendritic spines to reinforce a particular neural pathway. By strengthening the connection between two neurons, the ability of the presynaptic cell to activate the postsynaptic cell is enhanced. Several other mechanisms are also involved in learning- and memory-induced changes in synaptic structure, including changes in the amount of neurotransmitter released into a synapse and changes in how effectively cells respond to those neurotransmitters (Gaiarsa et al., 2002). Because memory is produced by interconnected networks of synapses in the brain, such changes provide the neurochemical foundations of learning and memory.

Changes in dendritic spine morphology are also associated with synaptic loss during ageing. The density of both excitatory (asymmetric) and inhibitory (symmetric) synapses in certain areas of the frontal cortex of Rhesus monkeys decreased by 30% from 5 to 30 years of age. Peters et al., Neuroscience, 2008, 152(4):970-81. This correlated with cognitive impairment. Similar synaptic loss has been observed in autopsies of Alzheimer's disease patients and is the best pathologic correlation to cognitive decline.

Another method of protecting against in vivo neurodegeneration is by activating neurotrophin production. Neurotrophins, particularly brain-derived neurotrophic factor (BDNF) and nerve growth factor (NGF), are key growth factors that initiate repair and regrowth of damaged neurons and synapses. Activation of some PKC isozymes, particularly PKC-ε and PKC-α, has been shown to protect against neurological injury, most likely by upregulating the production of neurotrophins. Weinreb et al., The FASEB Journal, 2004, 18:1471-1473). PKC activators are also reported to induce expression of tyrosine hydroxylase and induce neuronal survival and neurite outgrowth. Du and Iacovitti, J. Neurochem., 1997, 68: 564-69; Hongpaisan and Alkon, Proc Natl Acad Sci USA, 2007, 104:19571-19576; Lallemend et al., J. Cell Sci., 2005, 118:4511-25.

According to at least one embodiment of the present disclosure, therapeutically useful PKC activators reverse a decrease in dendritic spine density, such as by measuring the level of protein-marker spinophilin or synaptophysin, and preventing a decrease in pyramidal cells, mushroom spine-shape dendritic spines, and synapses, such as by using known measuring techniques in the art. According to another embodiment, in vivo studies with candidate PKC activators can be used to determine the candidate's effectiveness, for instance by evaluating performance in a quadrant test or memory retention trial to determine whether the candidate prevented learning and memory deficits. A positive result is found when the candidate PKC activator compounds result in a statistically significant increase in spinophilin or synaptophysin, or in pyramidal cells, dendritic spines and synapses.

Enhancement of Learning and Memory in Normal Animal Model

According to the present disclosure, therapeutically useful PKC activators enhance learning and memory in normal (i.e., healthy) animal models. As discussed in the section above, the formation of mushroom spines is known to provide structural storage sites for long-term associative memory and sites for memory-specific synaptogenesis. Thus, mushroom spine density may be used as another marker for identifying a PKC activator that enhances learning and memory in normal subjects and therefore, may be used to identify therapeutically useful PKC activators according to the methods herein. For example, measurement of mushroom spine density in healthy rat cells can be determined by known techniques in the art. In at least one embodiment, a candidate PKC activator that results in, for example, a statistically significant increase in the number or density of mushroom dendritic spines and synapses is a positive result.

Induction of Downstream Synaptogenic Biochemical Events

As discussed above, PKC activates neurotrophin production, for example, neurotrophins, particularly brain-derived neurotrophic factor (BDNF) and nerve growth factor (NGF). PKC activators also increase the relative amount of non-amyloidogenic soluble APP (sAPP) secreted by cells. For example, bryostatin-activation of PKC has been shown to activate the alpha-secretase that cleaves the amyloid precursor protein (APP) to generate the non-toxic fragments sAPP from human fibroblasts (Etcheberrigaray et al. (2004) Proc. Natl. Acad. Sci. 101:11141-11146).

According to the present disclosure, therapeutically useful PKC activators may induce downstream synaptogenic biochemical events, such as the induction of growth factors, for example NGF, BDNF, and IGF, and proteins such as GAP43, neurotrophin-3 (NT-3) sAPPα, and ELAV (ELAV proteins are generally involved in the post-transcriptional regulation of gene expression).

The presence of the protein MRNA of the neurotrophic factor may be used as a marker to identify therapeutically useful PKC activators according to the methods herein. Thus, a candidate PKC activator compound that results in, for example, a statistically significant increase in the level of NGF, BDNF, IGF, GAP43, neurotrophin-3 (NT-3), sAPPα, and ELAV, is a positive result.

Increases in Activity of A-β Degrading Enzymes

β-amyloid (“Aβ”) is a 4 kDa peptide produced by the proteolytic cleavage of amyloid precursor protein (“APP”) by β- and γ-secretases. Oligomers of Aβ are considered to be most toxic, while fibrillar Aβ is largely inert. Monomeric Aβ is found in normal patients and has an as-yet undetermined function.

It is known that PKC activators can reduce the levels of Aβ and prolong survival of AD transgenic mice. See Etcheberrigaray et al., 1992, Proc. Nat. Acad. Sci. USA, 89: 7184-7188. PKC-ε has been shown to be most effective at suppressing Aβ production. See Zhu et al., Biochem. Biophys. Res. Commun., 2001, 285: 997-1006. Accordingly, isoform-specific PKC activators are highly desirable as potential anti-AD drugs.

According to the present disclosure, therapeutically useful PKC activators may increase the activity of A-β degrading enzymes, such as ECE and neprilysin. The candidate PKC activator compounds that result in, for example, a statistically significant increase in the activity of neprilysin and ECE indicate a positive result.

Inhibition of GSK3β-Phosphorylation of Tau

PKC isozymes particularly -α and -ε, play a critical role in regulating the GSK-3β-mediated hyperphosphorylation of tau in neurofibrilliary tangles, and therefore, protect neurons from Aβ-mediated neurotoxicity, a major aspect of Alzheimer's disease pathology and Fragile X. Thus, GSK-3β is a key enzyme in the production of hyperphosphorylated tau protein, and phosphorylation of the Ser-9 residue causes GSK-3β inhibition, increasing phosphorylation of GSK-3β at Ser-9 by PKC could also enhance the protective effect of the PKC activators. Accordingly, a PKC activator that inhibits GSK3β-phosporylation of tau protein is a desirable characteristic for drug therapy.

According to the present disclosure, therefore, therapeutically useful PKC activators may inhibit GSK-3β phosphorylation of tau protein. The free GSK-3β protein and the phosphorylated GSK-3β protein can be used as markers for measuring increased phosphorylated GSK-3β. For example, candidate PKC activators that result in, for example, a statistically significant increase in phosphorylated GSK-3β is a positive result

Activation of α-Secretase

PKC activation results in an enhanced or favored a-secretase, non-amyloidogenic pathway. Therefore PKC activation is an attractive approach for activating the α-secretase pathway for the production of non-deleterious sAPP.

According to the present disclosure, therefore, therapeutically useful PKC activators may activate the α-secretase pathway. The level of sAPP-α protein can be used as a marker for measuring activated α-secretase. For instance, candidate PKC activators that result in, for example, a statistically significant increase in the level of sAPP-α protein indicates a positive result.

Certain embodiments provided herein can be illustrated by the following Examples, which are not intended to limit the full extent of disclosure provided herein in any ways.

### Examples

### Non-Tumorgenicity—Ames Tests of Pkc Activators

Protocol:

AMES testing for bryostatin, cyclopropanated arachidonic acid, DCPLA, and DHACP6, shown in Tables 1-4 below, did not result in a statistically significant positive response. The tests results indicate that bryostatin, cyclopropanated arachidonic acid, DCPLA, and DHACP6 are not mutagenic and therefore non-carcinogenic.

TABLE 1
Bryostatin Mutagenic Potential in TA 100 cells & TA 1535 cells
TA100 Cells

| Plate | Negative | Positive |
|---|---|---|
| Blank | 96 | 0 |
| Background | 89 | 7 |
| Background | 81 | 15 |
| Pos. Control | 2 | 94 |
| Bryostatin 0.125 mM | 87 | 9 |
| Bryostatin 0.25 mM | 80 | 16 |
| Bryostatin 0.5 mM | 82 | 14 |
| Bryostatin 1 mM | 83 | 13 |

Result: Not mutagenic, p > 0.05

TABLE 2
Cyclopropanated Arachidonic Acid Mutagenic Potential in
TA 100 cells & TA 1535 cells
Cyclopropanated Arachidonic Acid

| TA 100 Cells | | | TA 1535 Cells | | |
|---|---|---|---|---|---|
| Plate | Negative | Positive | Plate | Negative | Positive |
| Blank | 96 | 0 | Blank | 96 | 0 |
| Background | 89 | 7 | Background | 96 | 0 |
| Background | 81 | 15 | Background | 95 | 1 |
| Pos. | 2 | 94 | Pos. | 6 | 90 |
| Control | | | Control | | |
| BR121 | 87 | 9 | BR121 | 95 | 1 |
| 0.125 mM | | | 0.125 mM | | |
| BR121 | 80 | 145 | BR121 | 93 | 3 |
| 0.05 mM | | | 0.05 mM | | |
| BR121 | 82 | 14 | BR121 | 94 | 2 |
| 0.5 mM | | | 0.5 mM | | |
| BR121 | 83 | 13 | BR121 | 93 | 3 |
| 1 mM | | | 1 mM | | |

Result: Not mutagenic, p > 0.05

TABLE 3
DCPLA Mutagenic Potential in TA 100 cells & TA 1535 cells

| | | |
|---|---|---|
| Plate | Negative | Positive |

TA100 Cells

| | | |
|---|---|---|
| Blank | 96 | 0 |
| Background | 89 | 7 |
| Brackground | 81 | 15 |
| Pos. Control | 2 | 94 |
| DCPLA (not ester) 0.125 mM | 87 | 9 |
| DCPLA (not ester) 0.25 mM | 80 | 16 |
| DCPLA (not ester) 0.5 mM | 82 | 14 |
| DCPLA (not ester) 1 mM | 83 | 13 |

TA1535 Cells

| | | |
|---|---|---|
| Blank | 96 | 0 |
| Background | 96 | 0 |
| Brackground | 95 | 1 |
| Pos. Control | 6 | 90 |
| DCPLA 0.125 mM | 95 | 1 |
| DCPLA 0.25 mM | 93 | 3 |
| DCPLA 0.5 mM | 94 | 2 |
| DCPLA 1 mM | 93 | 3 |

Result: Not mutagenic, p > 0.05

TABLE 4
DHA-CP6 Mutagenic Potential in TA 100 cells & TA 98 cells

| | | |
|---|---|---|
| Plate | Negative | Positive |

TA100 Cells

| | | |
|---|---|---|
| Blank | 96 | 0 |
| Background | 83 | 13 |
| Brackground | 86 | 10 |
| Pos. Control | 2 | 94 |
| DHA-CP6 0.0625 mM | 88 | 8 |
| DHA-CP6 0.125 mM | 86 | 10 |
| DHA-CP6 0.25 mM | 92 | 4 |
| DHA-CP6 0.5 mM | 94 | 2 |

TA98 Cells

| | | |
|---|---|---|
| Blank | 96 | 0 |
| Background | 95 | 1 |
| Brackground | 94 | 2 |
| Pos. Control | 2 | 94 |
| DHA-CP6 0.0625 mM | 95 | 1 |
| DHA-CP6 0.125 mM | 96 | 0 |
| DHA-CP6 0.25 mM | 94 | 2 |
| DHA-CP6 0.5 mM | 95 | 1 |

Result: Not mutagenic, p > 0.05

Non-Toxicity—Internal Toxicity Studies

Protocol

Internal tests measuring various biological markers were performed 24 hours after administering a PKC activator (bryostatin, cyclopropanated arachidonic acid, DCPLA, and DHACP6) at 10× the therapeutic dose. The results are shown below in Tables 5-7. The results indicate that bryostatin, cyclopropanated arachidonic acid, DCPLA, and DHACP6, did not demonstrate statistically significant differences in levels of biological markers as compared to normal levels, and therefore, qualify as non-toxic PKC activators.

TABLE 5
Clinical Chemistry Panel & Hematology Panel for
Cyclopropanated Arachidonic acid and Bryostatin
(150 μg/m2)

| | | |
|---|---|---|
| | Cyclopropanated | |
| Test | Arachidonic Acid | Bryostatin i.v. 150 μg/m² |

Clinical Chemistry Panel

| | | | |
|---|---|---|---|
| | Control | | |
| ALB | 100 ± 0.9 | 97.0 ± 2.6 | 91.1 ± 0.8** |
| ALT | 100 ± 5.5 | 92.7 ± 5.4 | 63.6 ± 3.6** |
| ALP | 100 ± 13.3 | 93.8 ± 6.6 | 62.8 ± 10.0 |
| AST | 100 ± 12.3 | 107.0 ± 10.3 | 75.2 ± 11.3 |
| CO2-LC | 100 ± 4.2 | 100.0 ± 5.4 | 91.1 ± 4.2 |
| TBILI | 100 ± 6.5 | 78.2 ± 6.5 | 78.2 ± 13.0 |
| CA | 100 ± 1.5 | 101.0 ± 3.0 | 89.2 ± 6.1 |
| CREAT | 100 ± 5.3 | 92.9 ± 5.2 | 80.7 ± 3.5* |
| GLU | 100 ± 3.6 | 84.8 ± 2.4* | 77.6 ± 8.4 |
| TPROT | 100 ± 14.4 | 91.3 ± 1.4 | 91.3 ± 2.8 |
| BUN | 100 ± 6.8 | 92.0 ± 6.7 | 117.0 ± 4.5 |
| NA | 100 ± 0.6 | 99.3 ± 0.6 | 94.1 ± 1.9* |
| K | 100 ± 9.8 | 82.9 ± 3.6 | 101.0 ± 3.6 |
| CL | 100 ± 0.5 | 98.1 ± 0.5 | 95.4 ± 2.0 |

Hematology Panel

| | | | |
|---|---|---|---|
| | Normal | | |
| WBC | 100 ± 40.9 | 136.0 ± 42.6 | 142.3 ± 57.3 |
| NE | 100 ± 51.7 | 148.3 ± 51.7 | 144.8 ± 55.1 |
| LV | 100 ± 31.0 | 124.1 ± 34.4 | 134.4 ± 55.1 |
| MO | 100 ± 33.3 | 66.6 ± 33.3 | 133.3 ± 50.0 |
| EO | 100 ± 22.5 | 500.0 ± 250.0 | 375.0 ± 250.0 |
| BA | 100 ± 60.0 | 400.0 ± 200.0 | 300.0 ± 200.0 |
| RBC | 100 ± 3.0 | 89.2 ± 3.0 | 95.3 ± 20.0 |
| Hb | 100 ± 4.5 | 91.7 ± 6.0 | 83.4 ± 19.5 |
| HCT | 100 ± 1.4 | 93.3 ± 2.3 | 97.3 ± 21.9 |
| MCV | 100 ± 1.6 | 105.0 ± 2.0 | 101.8 ± 2.8 |
| MCH | 100 ± 7.3 | 103.0 ± 10.7 | 86.7 ± 4.9 |
| MCHC | 100 ± 5.5 | 98.1 ± 8.3 | 85.1 ± 2.8 |
| RDW | 100 ± 1.7 | 92.0 ± 0.5* | 106.8 ± 6.2 |
| MPV | 100 ± 12.3 | 82.7 ± 3.7 | 96.2 ± 2.4 |
| PLT | 100 ± 64.8 | 164.5 ± 3.7 | 109.4 ± 43.9 |

Toxicity observations at 10X therapeutic levels for multiple routes of entry.
Values are normalized to Control.
n = 5-6 for each cohort,
*= p < 0.05 vs Control,
**= p < 0.01 vs Control
ALB = Albumin
CREAT = Creatine
WBC = White blood count
HCT = Hematocrit (Low = Anemia)
ALT = Alanine aminotransferase
GLU = Glucose
NE = Neutrophils
MCV = Mean Corpuscular volume
ALP = Alkaline phosphatase
TPROT = Total Proteins
LY = Lymphocytes (lymphocytopenia)
MCH = Mean Corpuscular hemoglobin
AST = Aspertate aminotransferase
TRIG = Triglycerides
MO = Monocytes
MCH = Mean Corpuscular Hemoglobin Conc.
C02-LC = Bicarbonate
BUN = Blood Urea Nitrogen
EO = Eosinophils
RDW = Red cell distribution with
TBILI = Total Billirubin
NA = Sodium
BA = Basophils
PLT = Platelet count (Thrombocytopenia)
CA = Calcium
K = Potassium
RBC = Red Blood Cells (Low = Anemia)
MPV = Mean platelet volume
CHOL = Cholesterol
CL = Chloride
Hb = Hemoglobin (Low = Anemia)

TABLE 6
Clinical Chemistry Panel & Hematology Panel for
DCPLA (10 mg/kg) and DHA-CP6 (10 mg/kg)

| | | |
|---|---|---|
| | DCP-LA | DHA-CP6 |
| Test | 10 mg/kg | 10 mg/kg |

Toxicity Study: Clinical Chemistry Results 10 × Therapeutic Dose

| | | | |
|---|---|---|---|
| | Control | | |
| AST | 100 ± 12.3 | 107 ± 10.3 | 89.6 ± 11.3 |
| ALT | 100 ± 5.45 | 92.7 ± 5.4 | 74.5 ± 3.6* |
| ALP | 100 ± 13.3 | 93.8 ± 6.6 | 79.8 ± 10.0 |
| TBILI | 100 ± 6.52 | 78.2 ± 6.5 | 100 ± 13.0 |
| GLU | 100 ± 3.61 | 84.8 ± 2.4* | 115.4 ± 8.4 |
| TPROT | 100 ± 14.4 | 91.3 ± 1.4 | 97.1 ± 2.8 |
| CREAT | 100 ± 5.26 | 92.9 ± 5.2 | 89.4 ± 3.5 |
| CA | 100 ± 1.53 | 101 ± 3.0 | 103.0 ± 6.1 |
| BUN | 100 ± 6.76 | 92 ± 6.7 | 102.2 ± 4.5 |
| CO2-LC | 100 ± 4.21 | 100 ± 5.4 | 84.3 ± 4.2 |
| ALB | 100 ± 0.882 | 97.0 ± 2.6 | 97.0 ± 0.8 |
| NA | 100 ± 0.645 | 99.3 ± 0.6 | 100.6 ± 1.9 |
| K | 100 ± 9.75 | 82.9 ± 3.6 | 108.5 ± 3.6 |
| CL | 100 ± 0.545 | 98.1 ± 0.5 | 100 ± 2.0 |

Toxicity Study: Hematology Results 10 × Therapeutic Dose

| | | | |
|---|---|---|---|
| | Normal | | |
| WBC | 100 ± 40.9 | 136.0 ± 42.6 | 157.3 ± 34.4 |
| NE | 100 ± 51.7 | 148.2 ± 51.7 | 155.1 ± 34.4 |
| LY | 100 ± 31.0 | 124.1 ± 34.4 | 158.6 ± 34.4 |
| MO | 100 ± 33.3 | 66.6 ± 33.3 | 100 ± 33.3 |
| EO | 100 ± 22.5 | 500 ± 250 | 250 ± 125 |
| BA | 100 ± 60 | 400 ± 200 | 600 ± 300 |
| RBC | 100 ± 3.0 | 89.2 ± 3.0 | 95.3 ± 4.6 |
| Hb | 100 ± 4.5 | 91.7 ± 6.0 | 95.4 ± 4.5 |
| HCT | 100 ± 1.4 | 93.3 ± 2.3 | 100 ± 4.3 |
| MCV | 100 ± 1.6 | 105 ± 2.0 | 104.9 ± 0.9 |
| MCH | 100 ± 7.3 | 103 ± 10.7 | 100.4 ± 6.3 |
| MCHC | 100 ± 5.5 | 98.1 ± 8.3 | 95.5 ± 5.4 |
| RDW | 100 ± 1.7 | 92 ± 0.5* | 97.1 ± 2.8 |
| PLT | 100 ± 64.8 | 164.5 ± 62.5 | 213.1 ± 18.0 |
| MPV | 100 ± 12.3 | 82.7 ± 3.7 | 320.9 ± 246.9 |

AST—Aspartate aminotransferase (SGOT)
ALT—Alkaline aminotransferase (SGPT)
ALP—Alkaline phosphatase
TBILI—Total bilirubin
GLU—Glucose
TPROT—Total protein
CREAT—Creatinine
CA—calcium
BUN—Blood urea nitrogen
CO2-L—Bicarbonate
ALB—Albumin
NA—sodium
K—potassium
CL—chloride
*p < 0.05,
**p < 0.01
Leukocytes
WBC—White blood cells
NE Neutrophils
LY Lymphocytes (lymphocytopenia)
MO Monocytes
EO Eosinophils
BA Basophils
Thrombocytes
PLT—Platelet Count (Thrombocytopenia)
MPV Mean platelet volume
Erythrocytes
RBC Red Blood Cells (Low - anemia)
Hb Hemoglobin
HCT Hematocrit (Low - anemia)
MCV Mean corpuscular volume
MCH Mean corpuscular hemoglobin
MCHC Mean corpuscular hemoglobin concentration
RDW Red cell distribution width

TABLE 7
Clinical Chemistry Panel & Hematology Panel for various PKC activators

| | | | |
|---|---|---|---|
| Test | AA-CP4 10 mg/kg | DHA-CP6 10 mg/kg | Bryo 150 μg/m² |

Clinical Chemistry Results 10 × Therapeutic Dose

| | | | | |
|---|---|---|---|---|
| | Control | | | |
| AST | 100 ± 12.3 | 107 ± 10.3 | 89.6 ± 11.3 | 75.2 ± 11.3 |
| ALT | 100 ± 5.45 | 92.7 ± 5.4 | 74.5 ± 3.6* | 63.6 ± 3.6 |
| ALP | 100 ± 13.3 | 93.8 ± 6.6 | 79.8 ± 10.0 | 62.8 ± 10.0 |
| TBILI | 100 ± 6.52 | 78.2 ± 6.5 | 100 ± 13.0 | 78.2 ± 13.0 |
| GLU | 100 ± 3.61 | 84.8 ± 2.4* | 115.4 ± 8.4 | 77.8 ± 8.4 |
| TPROT | 100 ± 14.4 | 91.3 ± 1.4 | 97.1 ± 2.8 | 91.3 ± 2.8 |
| CREAT | 100 ± 5.26 | 92.9 ± 5.2 | 89.4 ± 3.5 | 80.7 ± 3.5 |
| CA | 100 ± 1.53 | 101 ± 3.0 | 103.0 ± 6.1 | 89.2 ± 6.1 |
| BUN | 100 ± 6.76 | 92 ± 6.7 | 102.2 ± 4.5 | 117. ± 4.5 |
| CO2-LC | 100 ± 4.21 | 100 ± 5.4 | 84.3 ± 4.2 | 91.1 ± 4.2 |
| ALB | 100 ± 0.882 | 97.0 ± 2.6 | 97.0 ± 0.8 | 91.1 ± 0.8 |
| NA | 100 ± 0.645 | 99.3 ± 0.6 | 100.6 ± 1.9 | 94.1 ± 1.9 |
| K | 100 ± 9.75 | 82.9 ± 3.6 | 108.5 ± 3.6 | 101. ± 3.6 |
| CL | 100 ± 0.545 | 98.1 ± 0.5 | 100 ± 2.0 | 95.4 ± 2.0 |

Hematology Results 10 × Therapeutic Dose

| | | | | |
|---|---|---|---|---|
| | Normal | | | |
| WBC | 100 ± 40.9 | 136.0 ± 42.6 | 157.3 ± 34.4 | 142.6 ± 57.3 |
| NE | 100 ± 51.7 | 148.2 ± 51.7 | 155.1 ± 34.4 | 144.81 ± 55.1 |
| LY | 100 ± 31.0 | 124.1 ± 34.4 | 158.6 ± 34.4 | 134.4 ± 55.1 |
| MO | 100 ± 33.3 | 66.6 ± 33.3 | 100 ± 33.3 | 133.3 ± 50 |
| EO | 100 ± 22.5 | 500 ± 250 | 250 ± 125 | 375 ± 250 |
| BA | 100 ± 60 | 400 ± 200 | 600 ± 300 | 300 ± 200 |
| RBC | 100 ± 3.0 | 89.2 ± 3.0 | 95.3 ± 4.6 | 95.3 ± 20 |
| Hb | 100 ± 4.5 | 91.7 ± 6.0 | 95.4 ± 4.5 | 83.4 ± 19.5 |
| HCT | 100 ± 1.4 | 93.3 ± 2.3 | 100 ± 4.3 | 97.3 ± 21.9 |
| MCV | 100 ± 1.6 | 105 ± 2.0 | 104.9 ± 0.9 | 101.8 ± 2.8 |
| MCH | 100 ± 7.3 | 103 ± 10.7 | 100.4 ± 6.3 | 86.7 + 4.9 |
| MCHC | 100 ± 5.5 | 98.1 ± 8.3 | 95.5 ± 5.4 | 85.1 ± 2.8 |
| RDW | 100 ± 1.7 | 92 ± 0.5* | 97.1 ± 2.8 | 106.8 + 6.2 |
| PLT | 100 ± 64.8 | 164.5 ± 62.5 | 213.1 ± 18.0 | 109.4 ± 43.9 |
| MPV | 100 ± 12.3 | 82.7 ± 3.7 | 320.9 ± 246.9 | 96.21 ± 2.4 |

AST = Aspartate aminotransferase (SGOT)
ALT = Alkaline aminotransferase (SGPT)
ALP = Alkaline phosphatase
TBILI = Total bilirubin
GLU = Glucose
TPROT = Total protein
CREAT = creatinine
BUN = Blood urea nitrogen
C02-L = Bicarbonate
ALB = Albumin
NA = Sodium
K = Potassium
CL = Chloride
*= p < 0.05
**= p < 0.01
Leukocytes
WBC—White blood cells
NE Neutrophils
LY Lymphocytes (lymphocytopenia)
MO Monocytes
EO Eosinophils
BA Basophils
Thrombocytes
PLT—Platelet Count (Thrombocytopenia)
MPV Mean platelet volume
Erythrocytes
RBC Red Blood Cells (Low - anemia)
Hb Hemoglobin
HCT Hematocrit (Low - anemia)
MCV Mean corpuscular volume
MCH Mean corpuscular hemoglobin
MCHC Mean corpuscular hemoglobin concentration
RDW Red cell distribution width

Brain Accessibility

Single IV Injections of Bryostatin

Protocol:

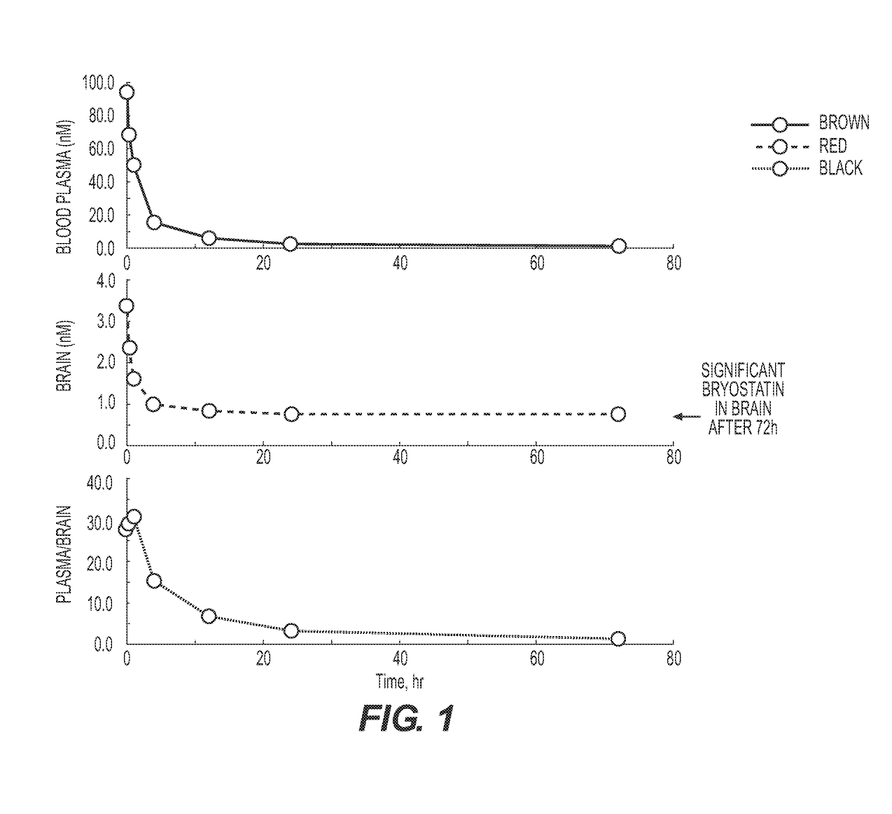

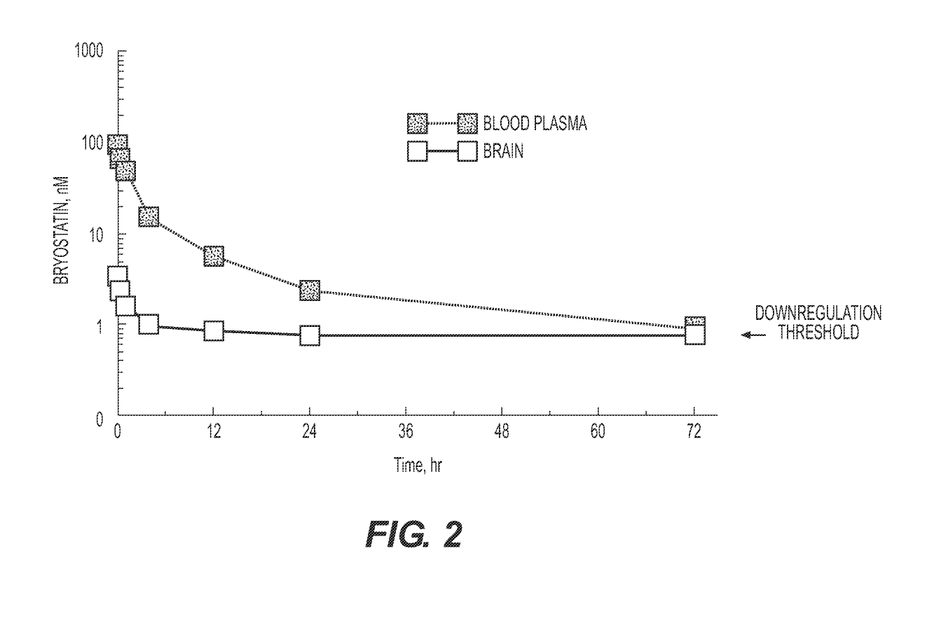

Measurements of bryostatin were analyzed at different time points subsequent to administration of a high dose of bryostatin (114 μg/m2). As shown in the middle curve in FIG. 1 below, bryostatin has an extremely long half-life in the brain as compared to in plasma. The plasma/brain ratio can be greater than 30. In addition, as shown in FIG. 2, brain bryostatin is below PKC downregulation in comparison to pla

[symbol]

PKC-ε Activation by Bryostatin in Mouse Brain

Protocol: Male C57BL/6M mice (15-20 g, Charles River) were acclimatized for 7-8 days in a non-enriched environment, three mice per cage. Bryostatin (Tocris) was dissolved in DMSO, diluted into 0.9% saline, and injected into the tail vein at doses of 10 and 15 μg/m2. After a fixed period, the mice were anesthetized with CO2 and the brain was frozen on dry ice. Blood was mixed with 0.2 ml 1 mM EDTA in PBS, centrifuged at 100 g for 30 min, and plasma was frozen on dry ice. In some experiments, blood lymphocyte fractions were collected using Ficoll-Paque Plus reagent using the procedure recommended by the manufacturer. All animal procedures were approved by the institutional IACUC.

Activation and translocation of PKC-ε were measured by Western blotting after subcellular fractionation into cytosol and particulate fractions. Homogenates were centrifuged at 100,000×g for 20 min and cytosolic and particulate fractions were separated on 4-20% Tris-glycine SDS polyacrylamide gels, blotted onto nitrocellulose, and probed with isozyme specific antibodies. The blots were photographed in a GE ImageQuant at 16 bits/pixel and analyzed by vertical strip densitometry using Imal Unix software.

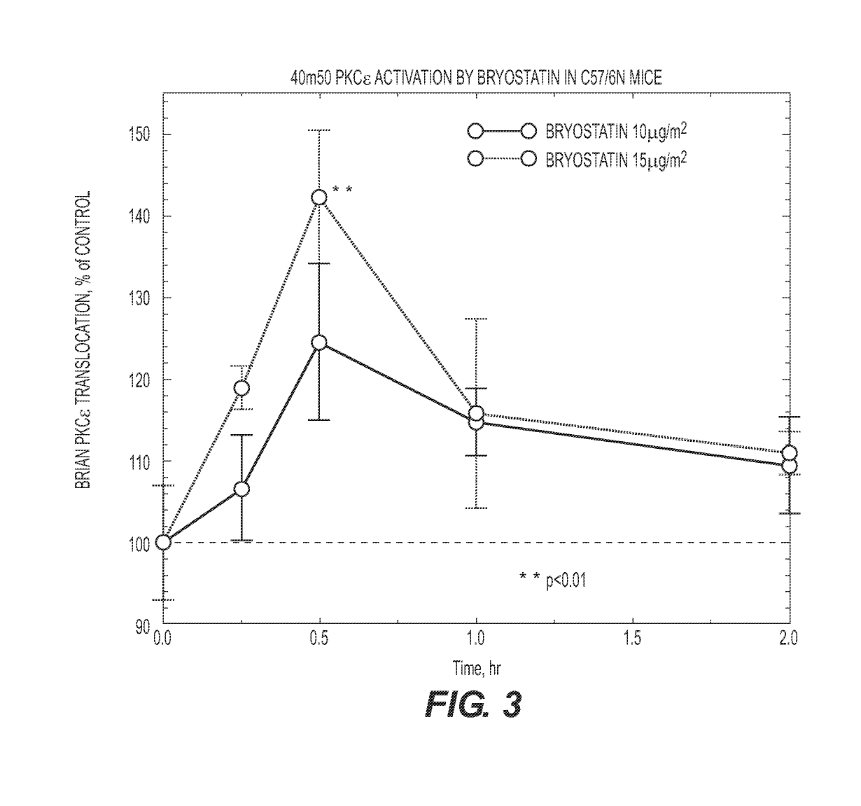

Bryostatin was injected into the tail vein of C57BL/6N mice at 10 and 15 μg/m2 (equivalent to 3.50 and 5.25 μg/kg), and brain PKC-ε concentration was measured using Western blots. Brain PKC-ε activation was biphasic, peaking at 0.5 h and slowly declined toward resting levels, even though bryostatin levels continued to increase. This is consistent with the short-lived activation of PKC established previously. No downregulation below starting values was observed. The bryostatin concentration at 0.5 h was 0.029 nM. The results are shown in FIG. 3 below.

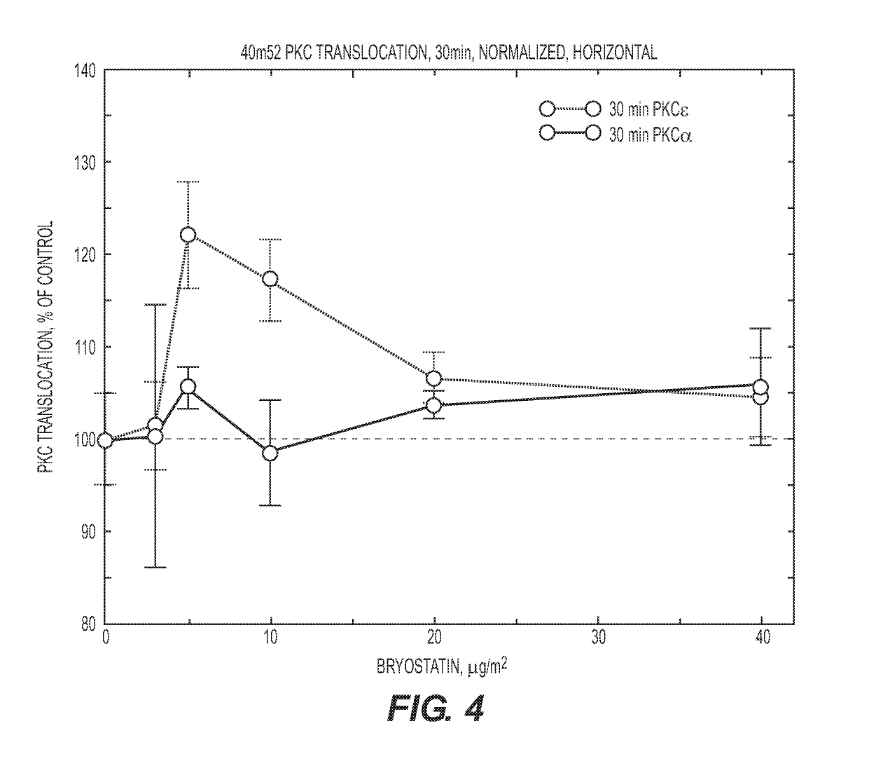

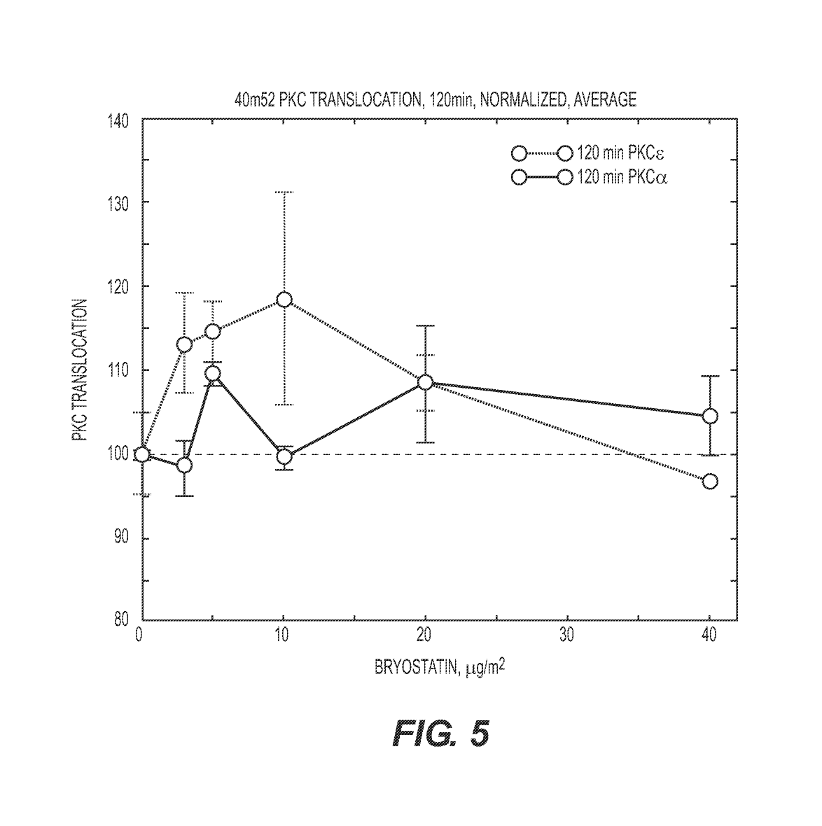

The results of a dose dependence study of activation of PKCα and PKCε translocation by bryostatin, are shown below in FIG. 4 (measured 30 minutes after administration), and FIG. 5 (measured 120 minutes after administration). The effect of bryostatin on brain PKC translocation (an indicator of enzymatic activation) was also biphasic, with maximal effects observed at doses between 5 and 10 μg/m2. In contrast to in vitro measurements with purified PKC isozymes, for which bryostatin activates the α isoform and ε isoform equally, in mouse brain, translocation was only observed by PKC-ε.

PKC-α and PKC-ε Specificity

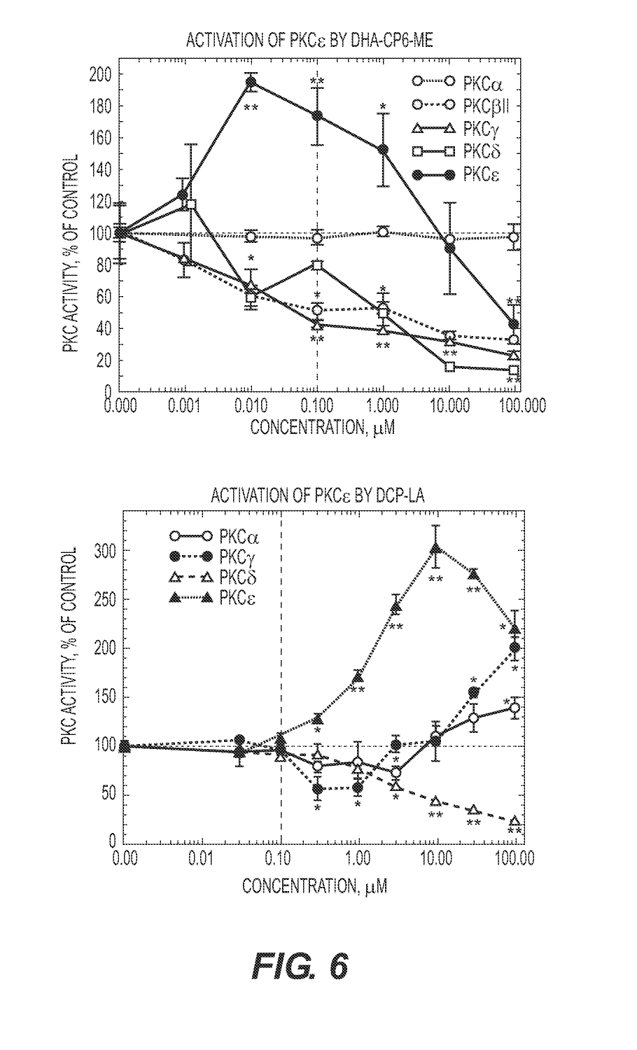

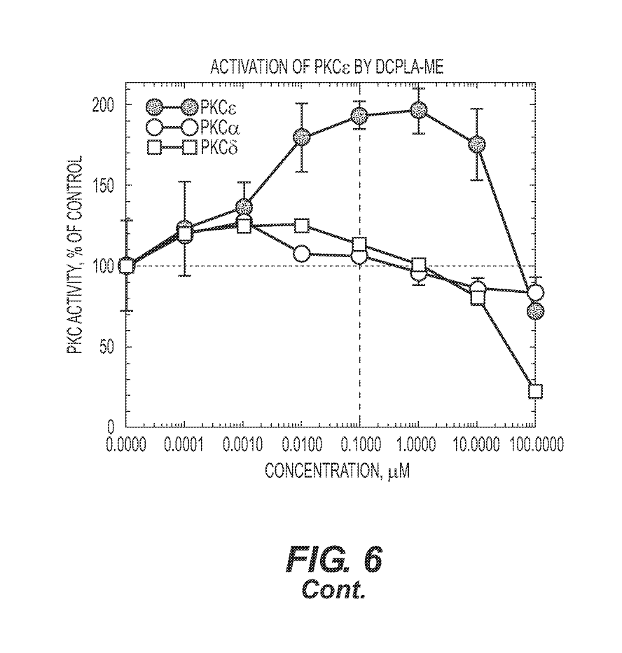

Protocol: Purified PKC-α, βII, γ, δ, or ε (9 ng) was preincubated for 5 minutes at room temperature with the following PKC activators: (A) DHA-CP6, (B) EPA-CP5, (C) AA-CP4, (D) DCP-LA, (E) “other cyclopropaneated and epoxidized fatty acids, alcohols, and methyl esters.” followed by measurement of PKC activity as described under Experimental Procedures. Results are shown below in FIG. 6. As shown in FIG. 6, DHA-CP6-methyl ester, DCP-LA, and DCPLA-methyl ester show a PKC-ε specificity ±15% PKC-α and PKC-δ.

Synaptogenicity

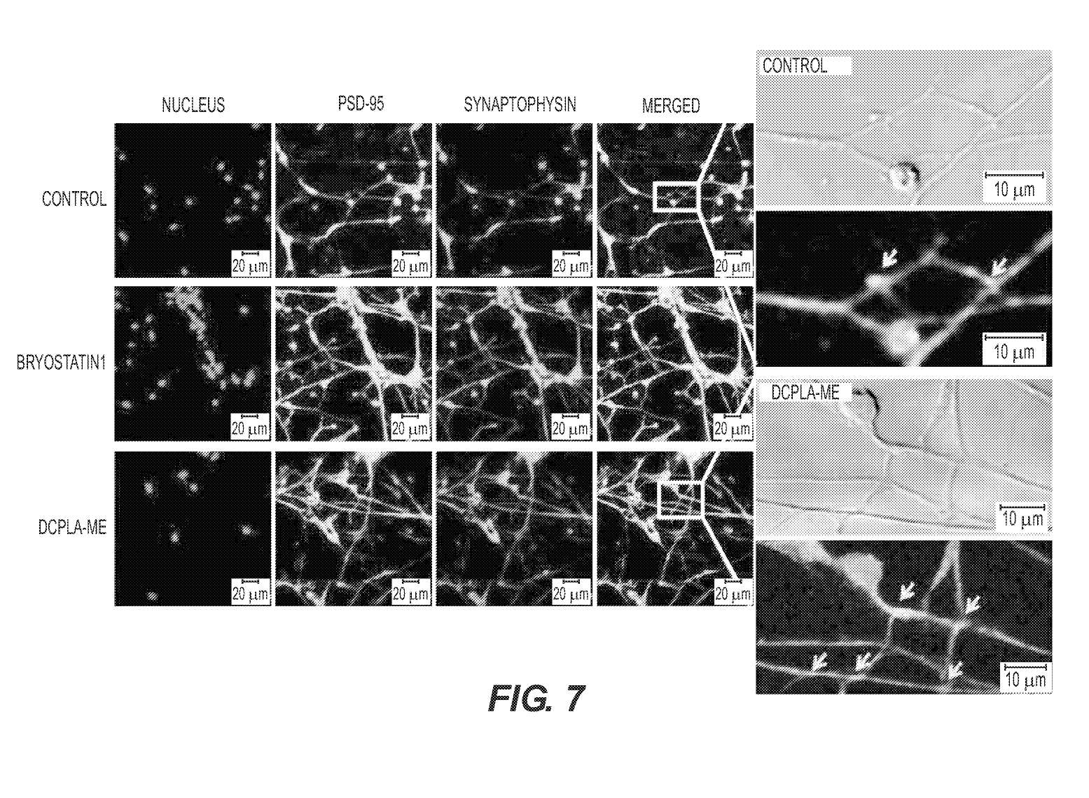

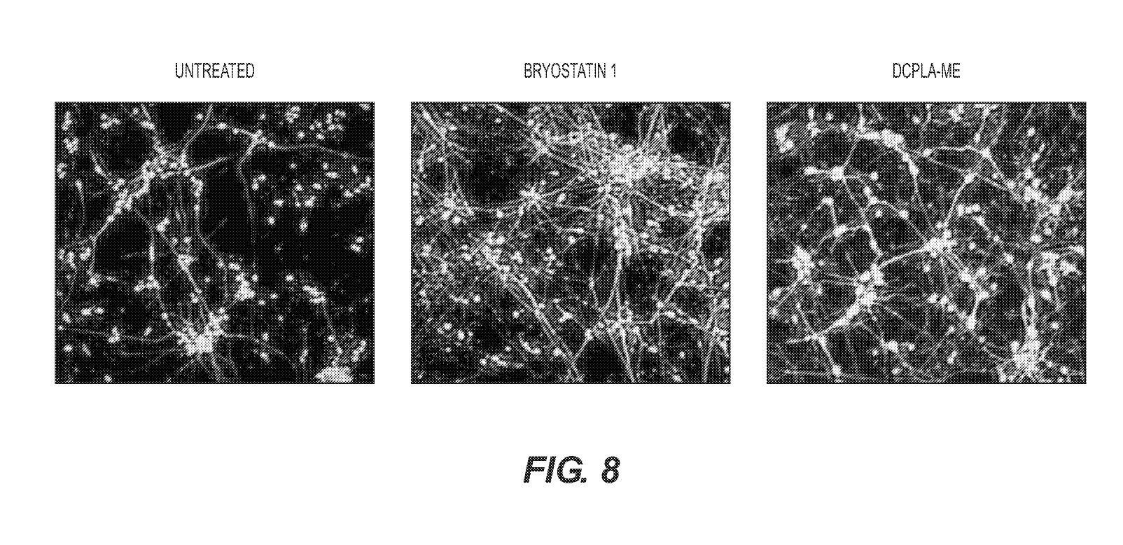

Protocol: Primary human neurons were treated with either DCPLA-methyl ester (100 nM) or bryostatin-1 (0.27 nM). As shown in FIG. 7, cells treated with either DCPLA-methyl ester or bryostatin-1 for 30 days showed an increase in co-localized staining of PSD-95 and synaptophysin in puncta, indicating an increase in the number of synapses (the figures to the right illustrate a typical synapse). As shown in FIG. 8, cells treated with either DCPLA-methyl ester or bryostatin-1 for 40 days showed an improved survival with increased neuritic branching and connections. In contrast, untreated cells showed degeneration after 20 days.

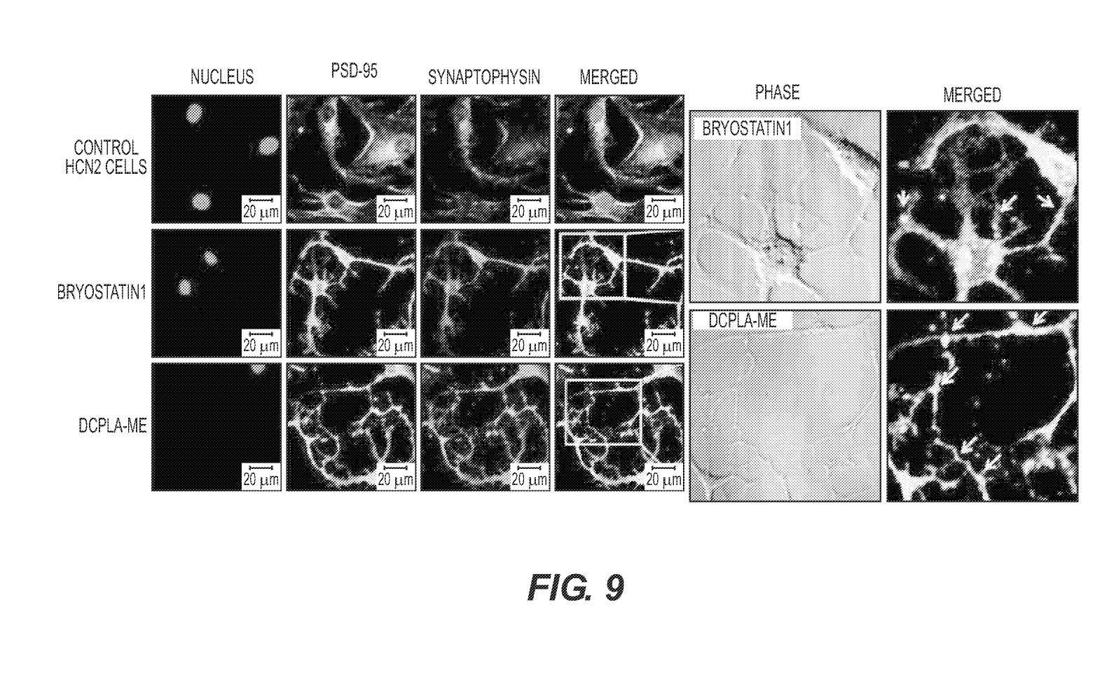

FIG. 9 illustrates that activation of PKC-ε induces synaptogenesis in HCN-2 cells. The HCN-2 cell line was derived from cortical tissue removed from a 7 year old patient undergoing hemispherectomy for intractable seizures associated with Rasmussen's encephalitis. The cells were treated with either DCPLA-methyl ester or bryostatin-1 for 10 days. As shown in FIG. 9, HCN-2 cells treated with either DCPLA-methyl ester or bryostatin-1 showed significant differentiation with neuronal branching and increased punctate colocalization of PSD-95 and synaptophysin indicating synapsin formation. Untreated cells showed fibroblast-like morphology without branching and punctate staining of PSD-95 and synaptophysin. Thus, PKC-ε activation can induce synaptogenesis in both embryonic and adult neuronal cells.

Anti-Apoptosis

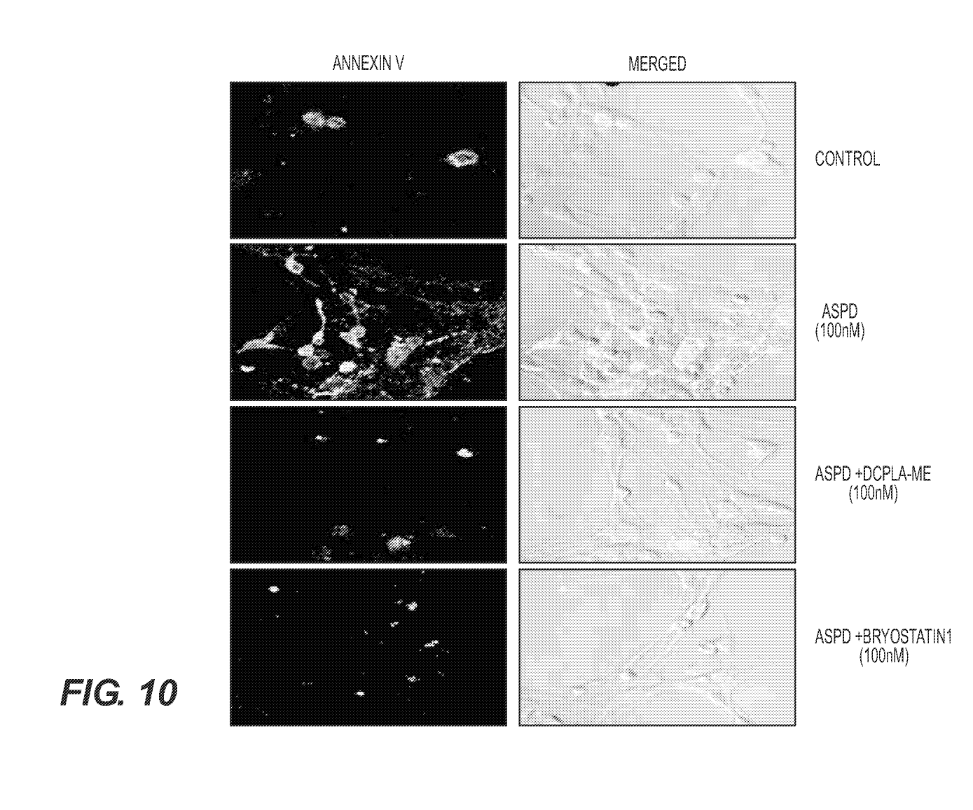

Protocol: Human primary neurons were grown on chambered slides and treated with vehicle (Control), 100 nM ASPD, ASPD+DCPLA-ME (100 nM), ASPD+bryostatin 1 (0.27 nM) and ASPD+DCPLA-ME (100 nM) or ASPD+bryostatin 1 (0.27 nM) in presence of PKC-ε inhibitor. Following 24 hours of incubation, cells were stained using Annexin-V Fluorescein to detect apoptotic cells and results are shown in FIG. 10 below. ASPD-induced apoptosis and PKC activators protected against ASPD-induced apoptosis. Data are mean±SEM of three independent experiments. (*p<0.05;** p<0.005 and *** p<0.0005).

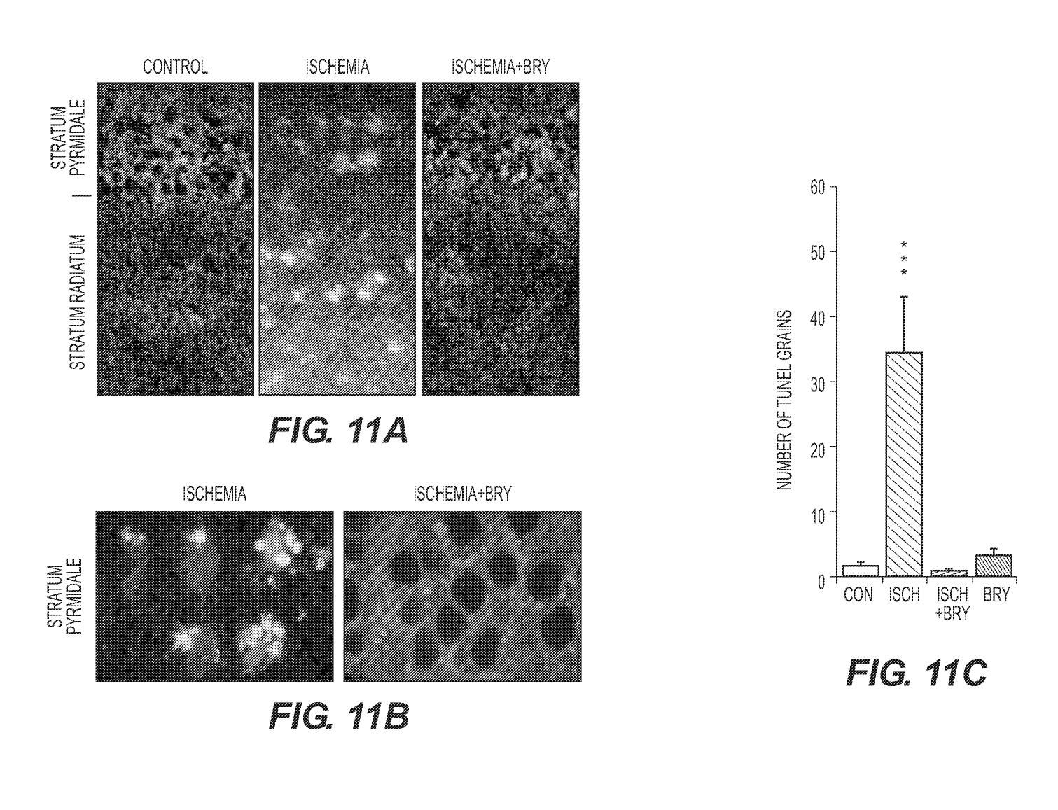

Protocol: Bryostatin-1 (15 μg/m²) was administered through a tail vein (2 doses/week, for 10 doses), starting 24 hours after the end of the ischemic (2-VO)/hypoxic event. Staining for apoptotic cell death in the hippocampal CA1 area was performed 9 day after the last bryostatin-1 dose. FIG. 11 below shows results of low (A) and high (B) magnification of apoptotic cell death in CA1 hippocampal area, detected by terminal deoxynucleotidyl transferase (TdT)-mediated dUTP nick end labeling (TUNEL) and visualized by a confocal microscope (a double-blind study). (C) Quantification of TUNEL staining in stratum radiatum (n=3 animals; n=30 confocal images). Con=control; Bry=bryostatin-1; Isch=cerebral ischemia; ***, P<0.001.

Neuroprotection Against Aspds

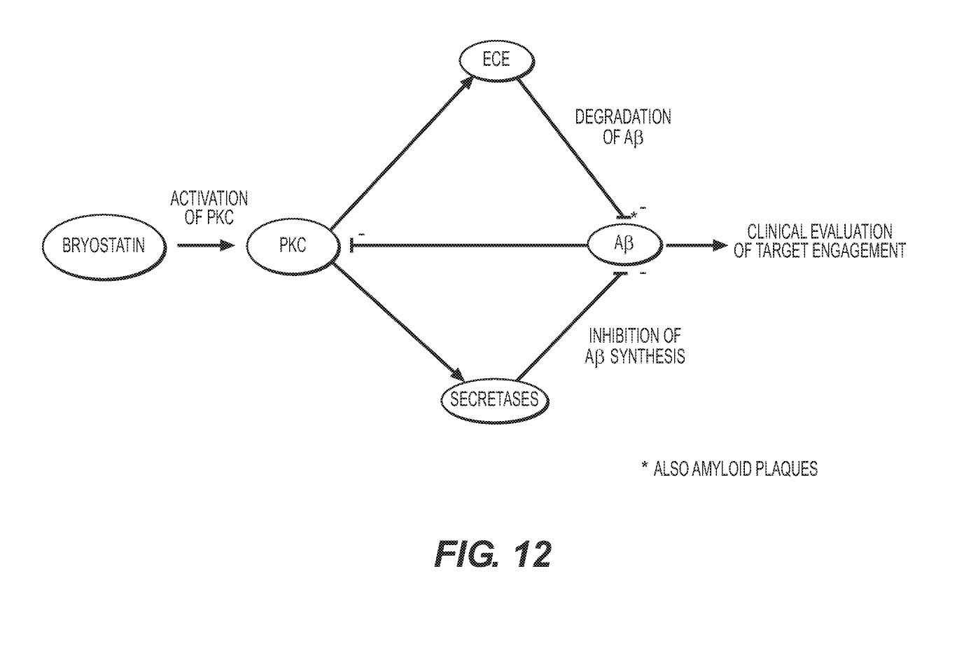

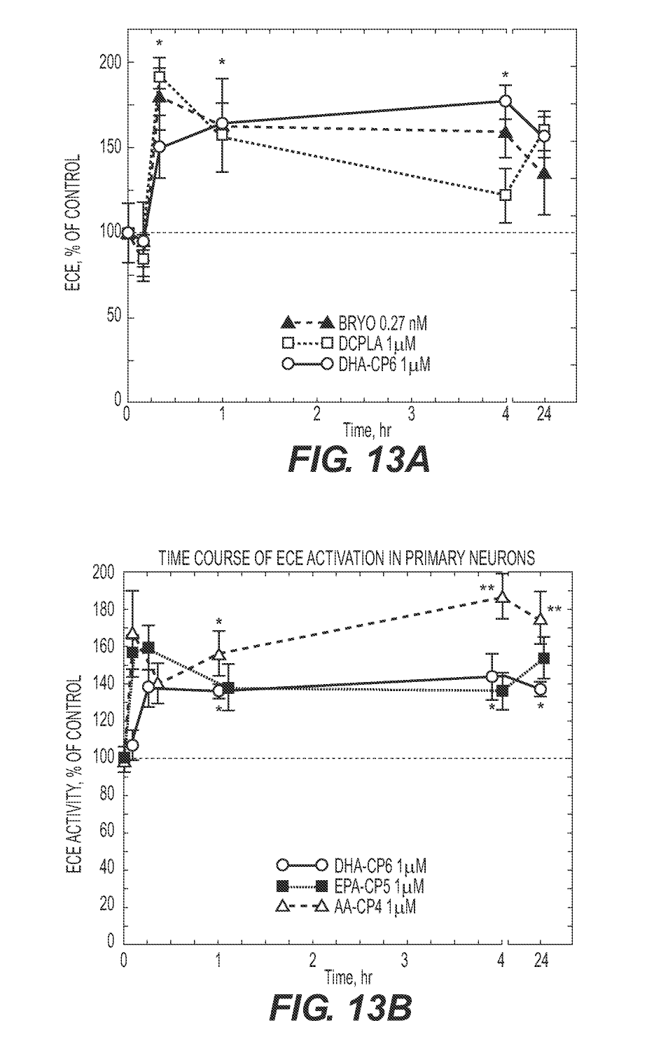

Aβ can be degraded in vivo by a number of enzymes, including insulin degrading enzyme (insulysin), neprilysin, and ECE (FIG. 12). Because PKC-ε overexpression has been reported to activate ECE (Choi et al., Proc. Natl. Acad. Sci. USA. 2006; 103: 8215-20), the effect of PKC activators on ECE was analyzed. Candidate PKC activators were added to either SH-SY5Y cells (Bryo—0.27 nM, DCP-LA—1 μM, and DHA-CP6 1 μM) or cultured neurons (DHA-CP6—1 μM, EPA-CP5— 1 μM, and AA-CP4— 1 μM), and grown on either 12- or 24-well plates. After various time periods, the cells were collected and ECE activity was measured fluorometrically. Results are shown below in FIG. 13. *, p<0.05; **, p<0.001. All test PKC activators produced an increase in ECE activity (as compared to ethanol alone). Since ECE does not possess a diacylglycerol-binding CI domain, this suggests that the activation by bryostatin was not due to direct activation of ECE, but must have resulted from phosphorylation of ECE or some ECE-activating intermediate by PKC. This result also suggests that indirect activation ECE by PKC activators could be a useful means of reducing the levels of Aβ in patients.

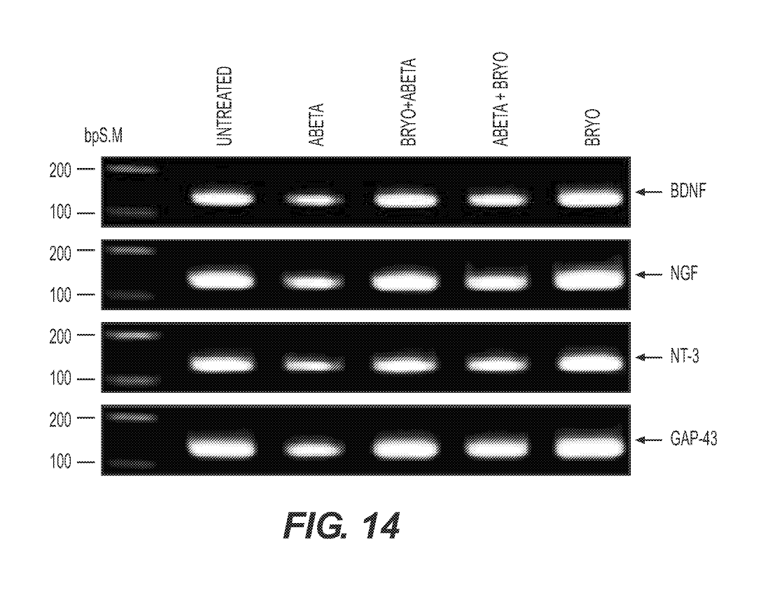

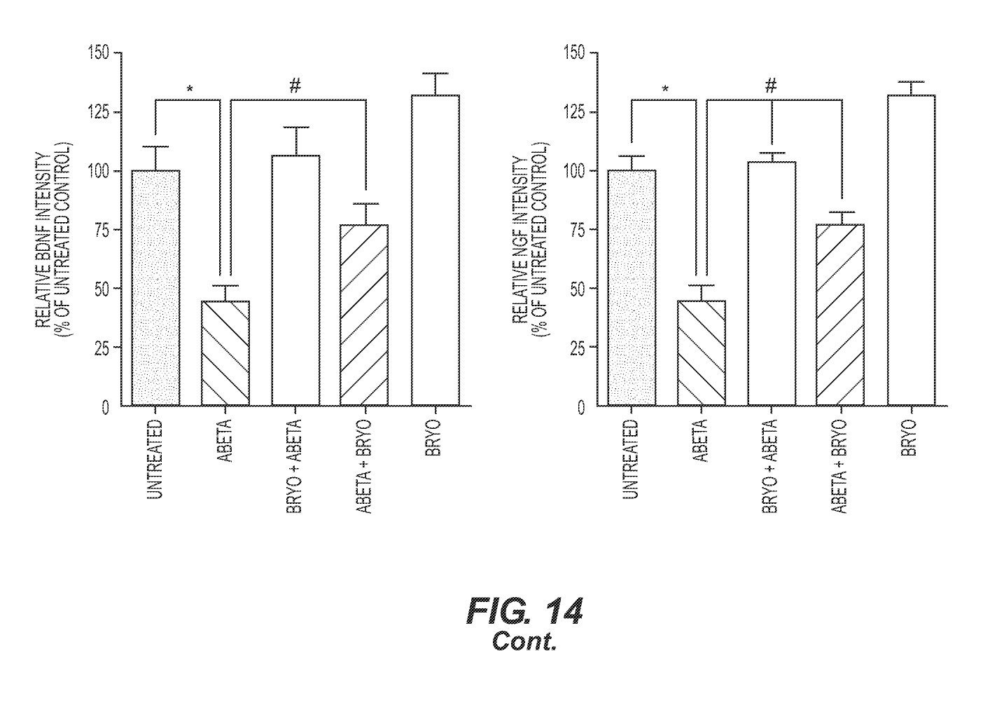

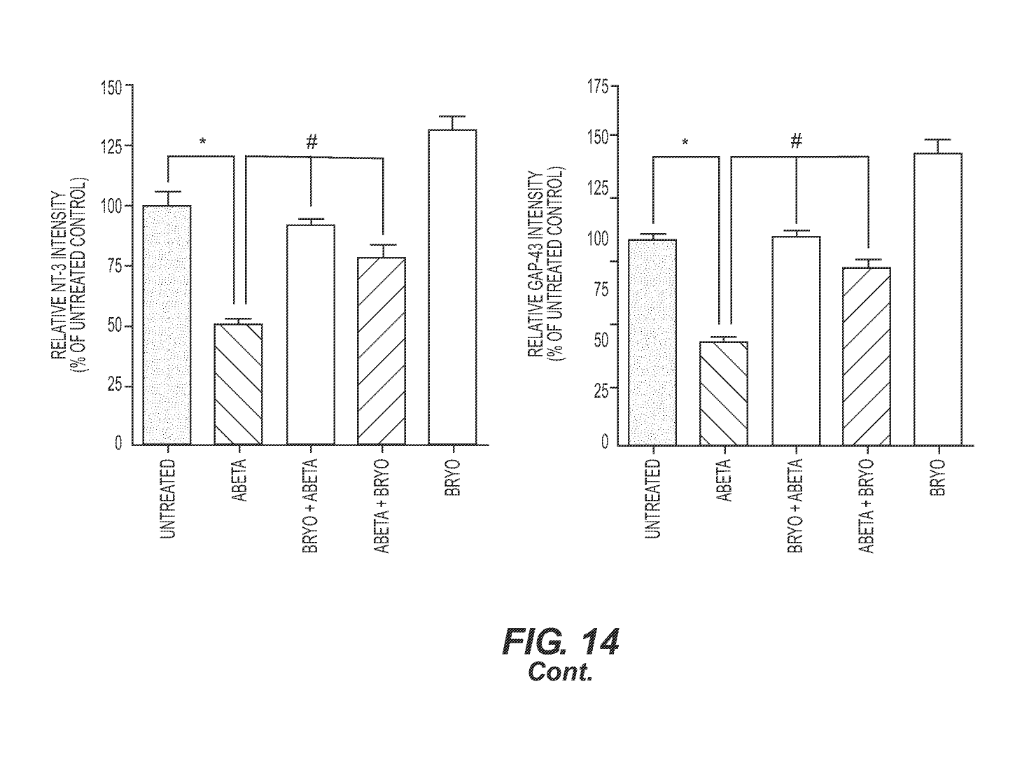

Protocol: Primary hippocampal neurons were treated with control buffer (Untreated), Aβ (1 μM, oligomeric form), or 0.5 nM bryostatin for 24 hours. Some cells were co-treated with Aβ plus 0.5 nM bryostatin (Bryo+Aβ) for 24 hours, or pre-treated with Aβ for 12 hours, washed out, and then treated with bryostatin for additional 12 hours (Aβ+Bryo). Cells were then lysed and total RNA was isolated. Relative expression change of BDNF, NGF, NT-3, and GAP-43 mRNA was quantitatively measured from real time PCR using specific primers against rat BDNF, NGF, NT-3, and GAP-43 mRNA (FIG. 14). A representative gel image is also shown below in FIG. 14 (Mean+SEM, *P<0.05, Aβ compared with untreated; #P<0.05, Bryo+Aβ or Aβ+Bryo compared with Aβ only).

Protocols:

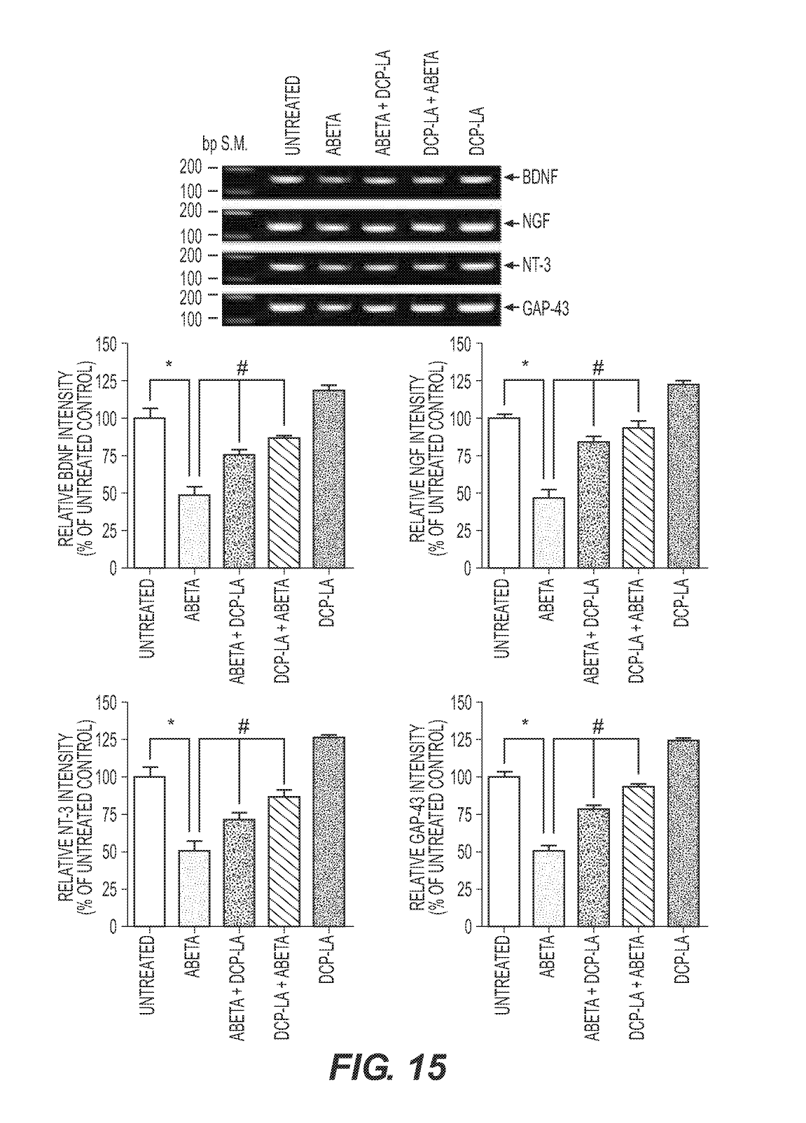

Test A—DCPLA (500 nM): Primary hippocampal neurons were treated with control buffer (Untreated), Aβ (1 μM, oligomer), or 500 nM DCPLA for 24 hours. Some cells were pre-treated with Aβ for 12 hr and then treated with 500 nM DCPLA for 12 hours (Aβ+DCP-LA), or co-treated with Aβ plus 500 nM DCP-LA (DCP-LA+Aβ) for 24 hours. Total RNA was isolated and relative expression change of BDNF, NGF, NT-3, and GAP-43 mRNA was quantitatively measured from real time RT-PCR using specific primers against rat BDNF, NGF, NT-3, and GAP-43 mRNA (FIG. 15). A representative gel image is also shown below in FIG. 15 (Mean+SEM of three independent experiments, *P<0.05, Aβ compared with untreated; #P<0.05, Aβ+Bryo or Bryo+Aβ compared with Aβ only).

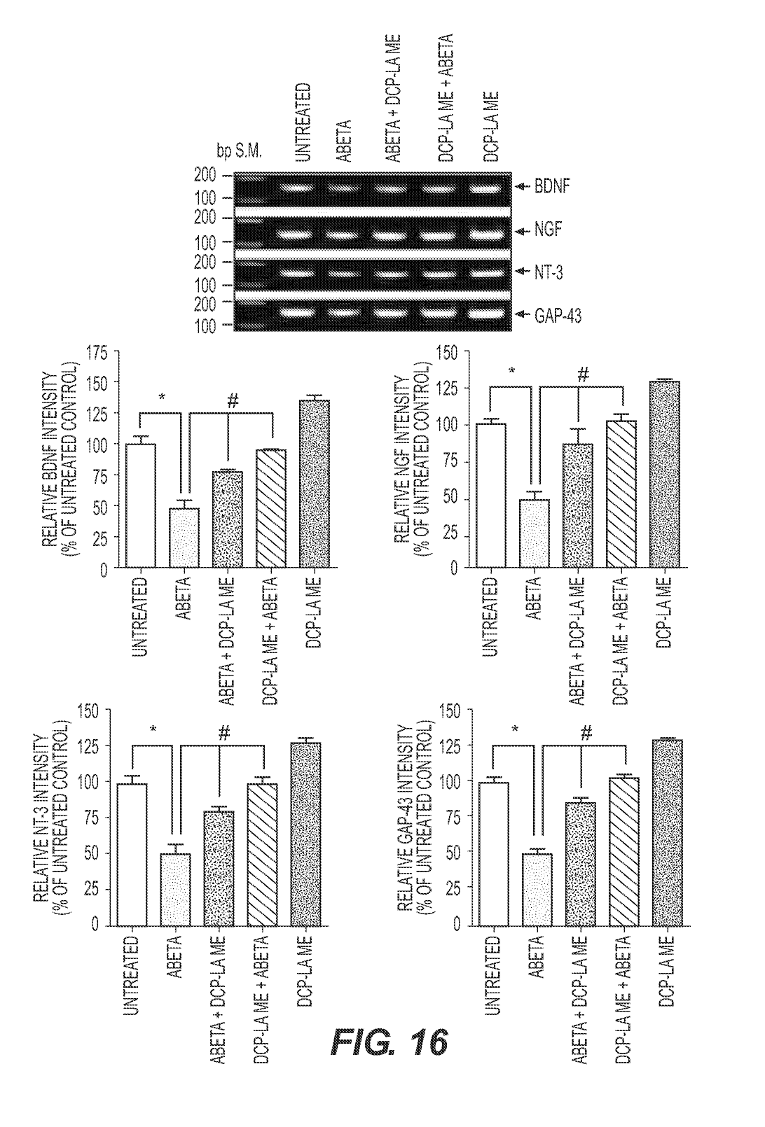

Test B—DCPLA-methyl ester (100 nM): Primary hippocampal neurons were treated with control buffer (Untreated), 1 μM Aβ, or 100 nM DCP-LA ME (the methyl ester form of DCP-LA) for 24 hours. Some cells were pre-treated with Aβ for 12 hr and then treated with 100 nM DCP-LA ME for 12 hours (Aβ+DCP-LA ME), or co-treated with Aβ plus 500 nM DCPLA ME (DCP-LA ME+Aβ) for 24 hours. Total RNA was isolated and relative expression change of BDNF, NGF, NT-3, and GAP-43 mRNA was quantitatively measured from real time RT-PCR using specific primers against rat BDNF, NGF, NT-3, and GAP-43 mRNA (FIG. 16). A representative gel image is also shown below in FIG. 16 (Mean+SEM of three independent experiments, *P<0.05, Aβ compared with untreated; #P<0.05, Aβ+Bryo or Bryo+Aβ compared with Aβ only).

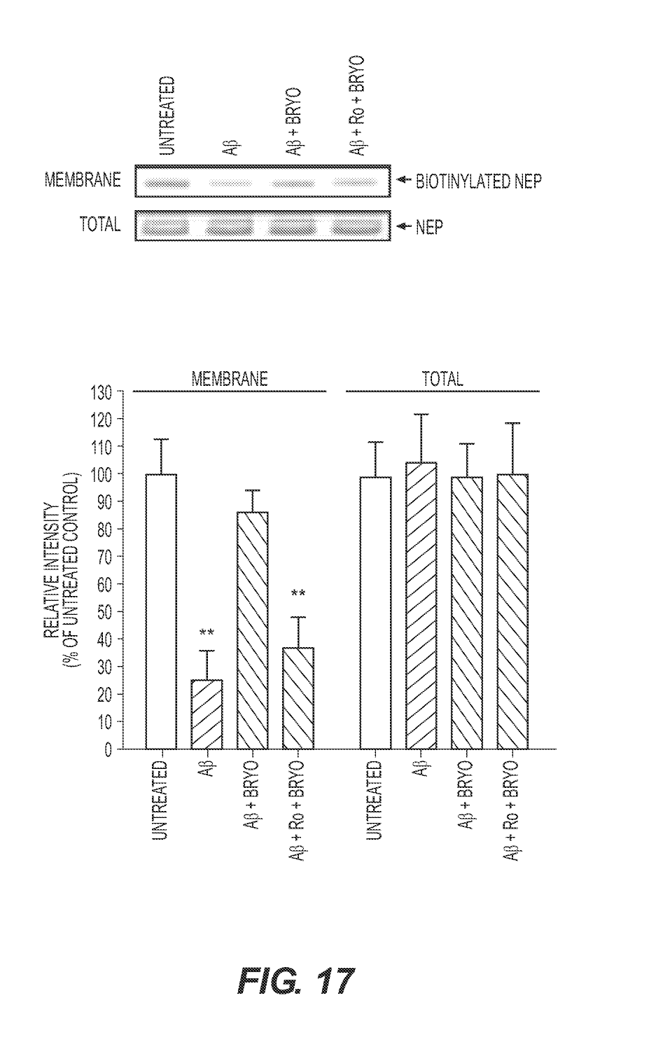

Protocols: SHSYSY cells overexpressing human neprilysin (SH+hNEP cells) were incubated with 1 μM oligomeric Aβ(1-42) for 4 hours in the absence or presence of bryostatin (Bryo, 1 nM). Some cells were pre-treated with Ro 32-0432 (Ro, 2 μM, a PKC inhibitor) for 30 min and then treated with Bryo. Cell surface-located proteins were then biotinylated and extracted using streptavidin beads, followed by immunoprecipitation using a neprilysin antibody Immunoprecipitates were subjected to Western blot analysis using neprilysin antibody (Mean+SEM of three independent experiments, **P<0.01, Aβ compared with untreated; #P<0.01, Ro+Bryo compared with Bryo). Results are shown below in FIG. 17.

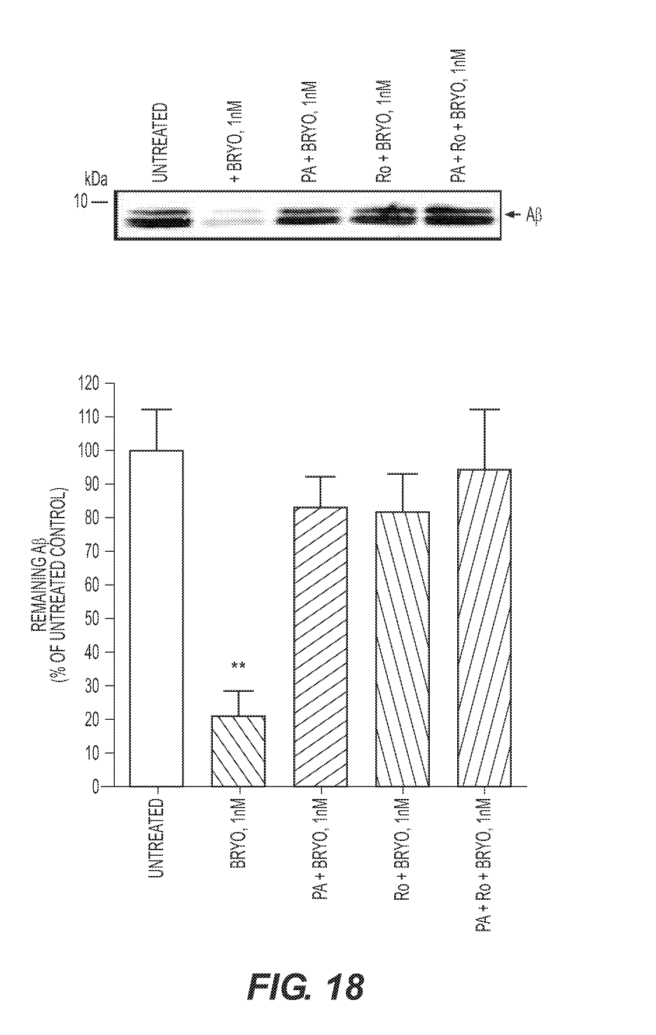

Protocol: Intact SH+hNEP cells were incubated with 2.5 μg of monomeric Aβ(1-42) for 4 hours in the absence or presence of bryostatin (Bryo, 1 nM). Some cells were cotreated with phosphoramidon (PA, 10 μM, a specific neprilysin inhibitor), Ro 32-0432 (Ro, 2 μM, a PKC inhibitor), or PA+Ro in the presence of bryostatin (1 nM) for 4 hours. Aβ peptide was then precipitated from the reactions by 20% trichloroacetic acid and immunoblotted with use of Aβ peptide 1-16 antibody (6E10). Arrow indicates the size of monomeric Aβ peptide (FIG. 18), which is around 4 kDa. Densitometry measurements of developed protein bands on Western blots were made and assigned as relative expression change (Mean+SEM of three independent experiments, **P<0.01, Bryo compared with untreated; #P<0.01, PA, Ro, or PA+Ro compared with Bryo.).

Protection Against In Vivo Neurodegeneration

Alzheimer's Disease:

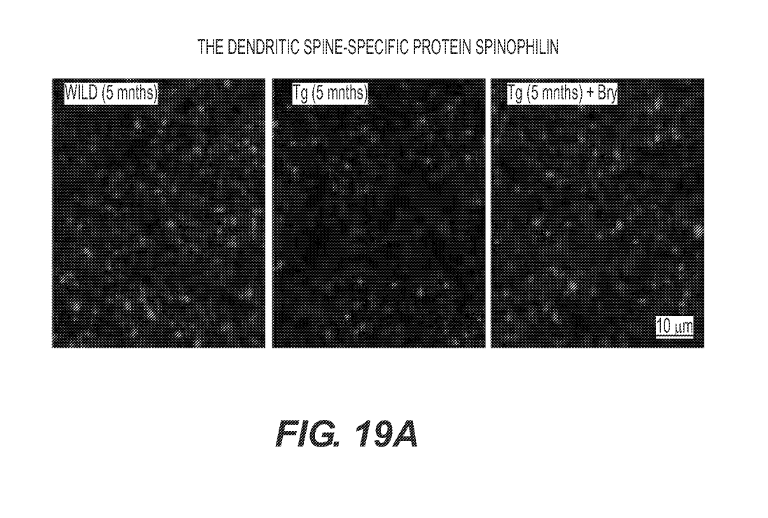

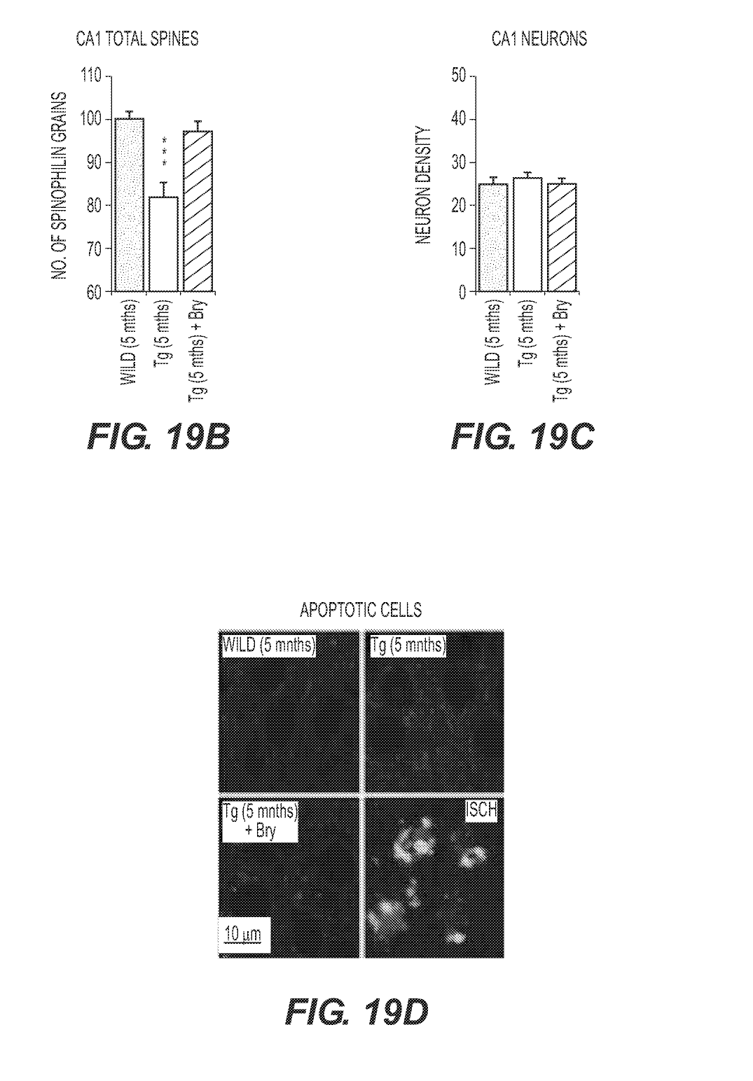

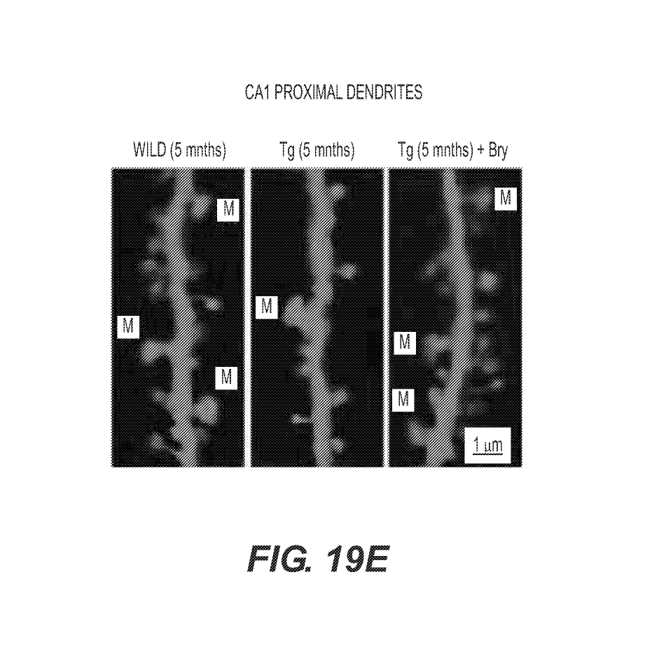

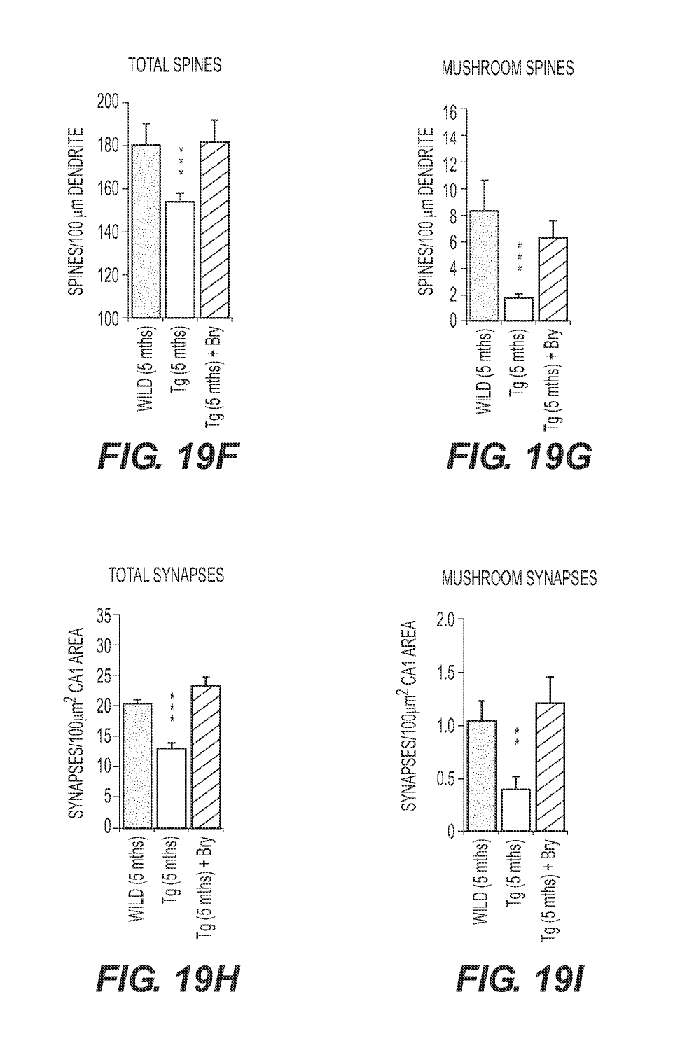

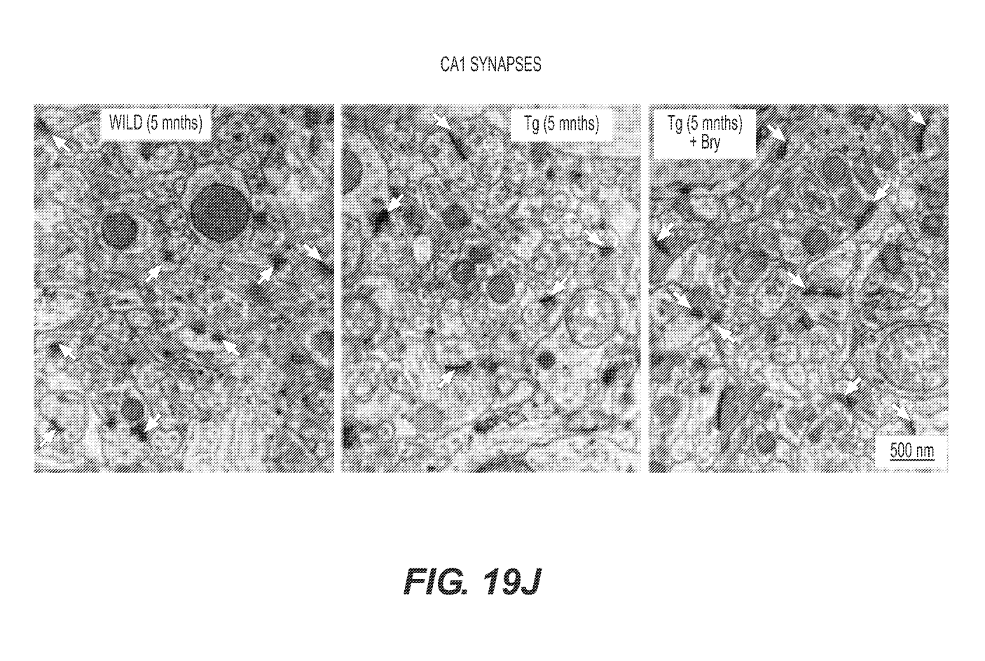

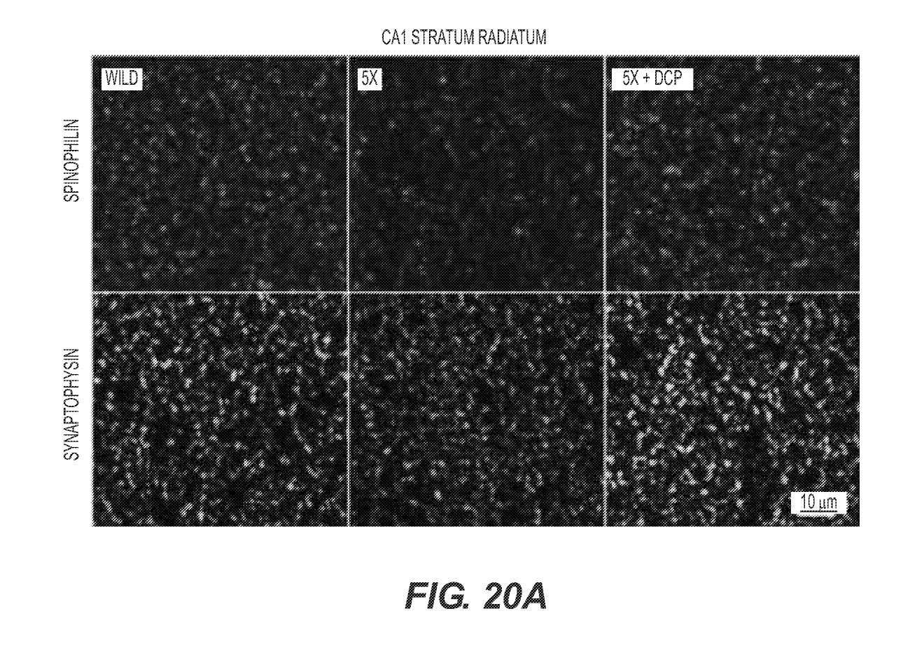

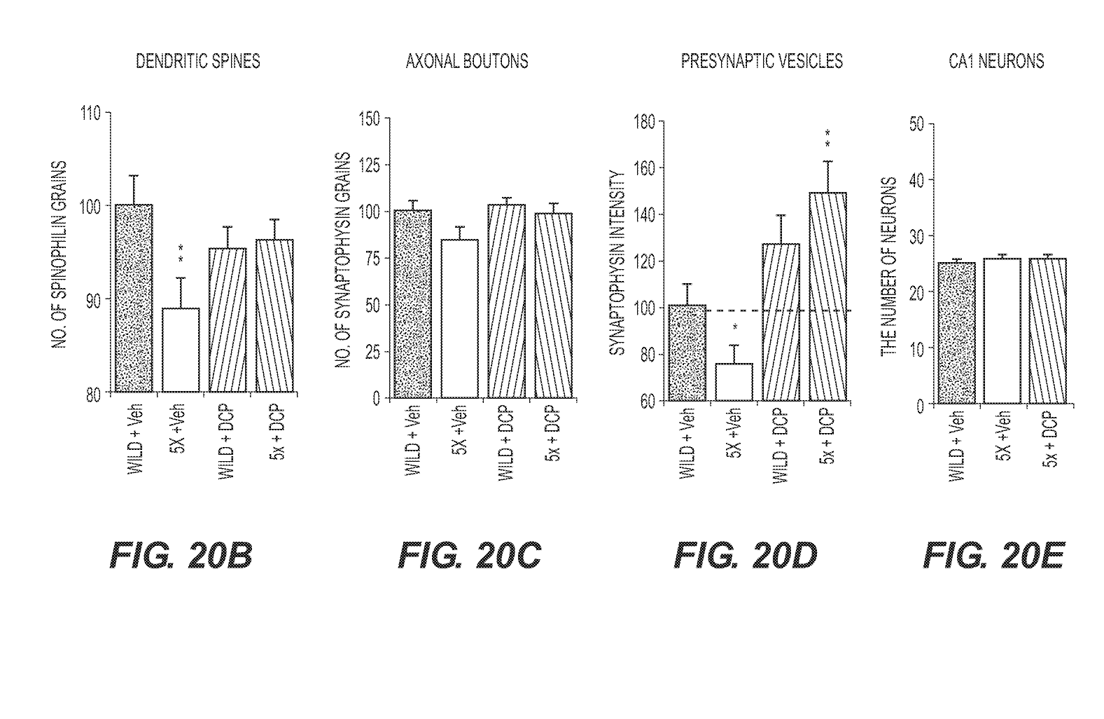

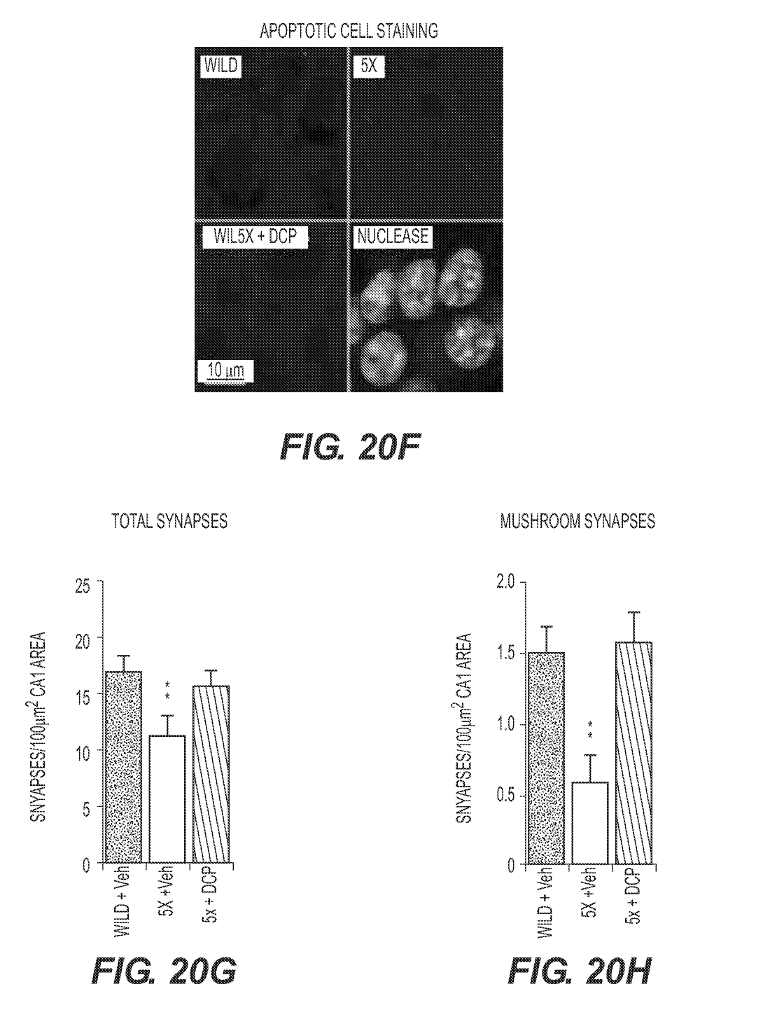

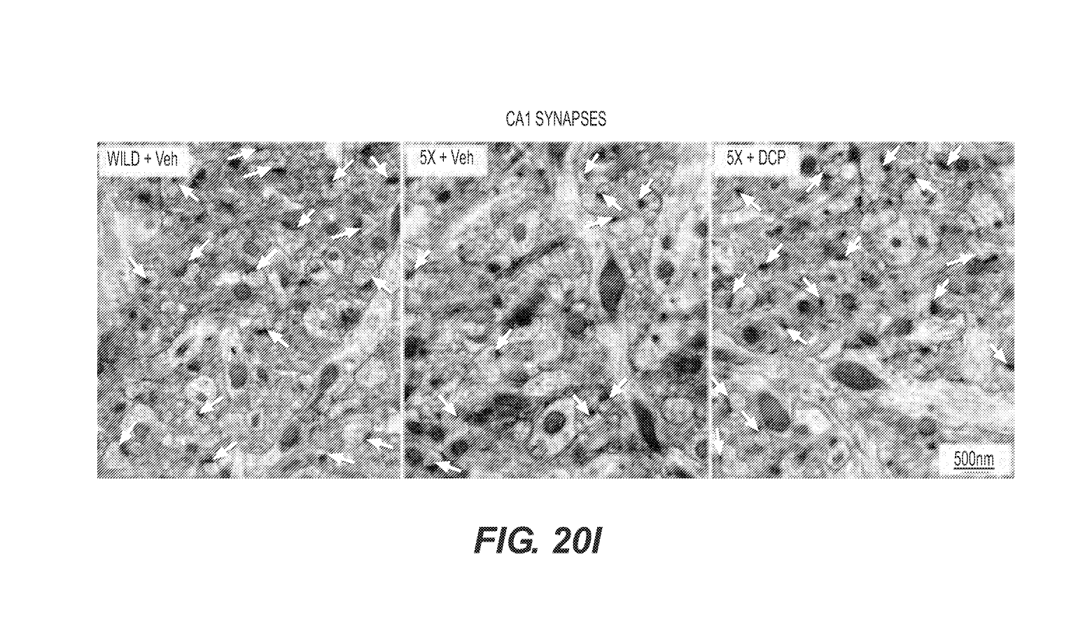

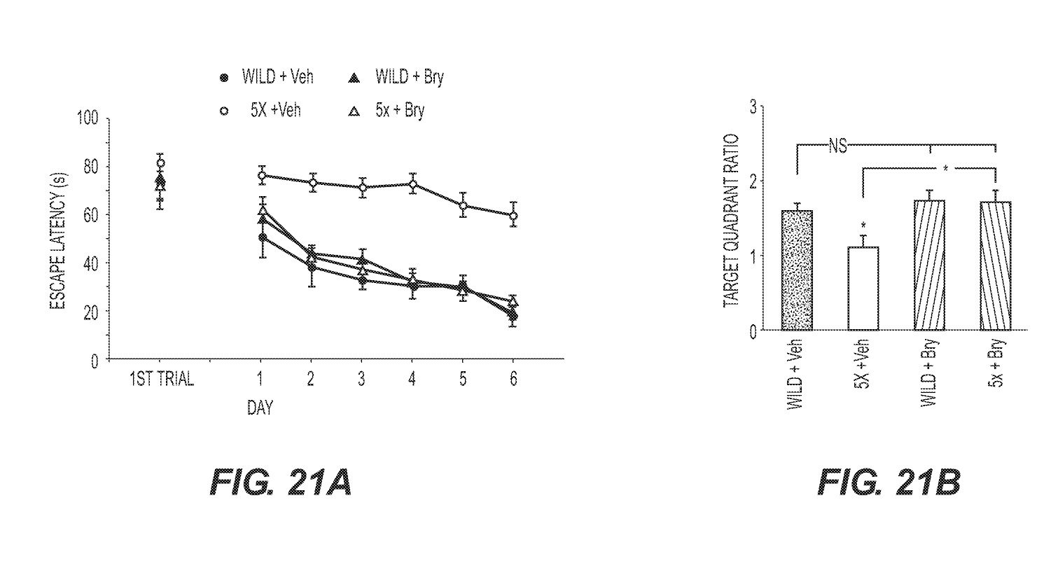

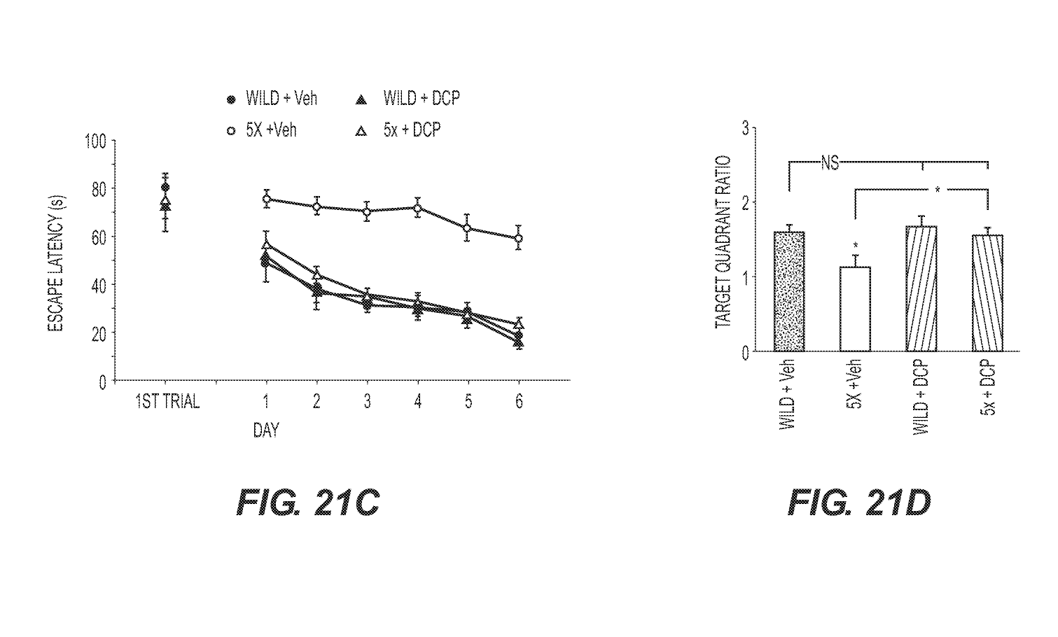

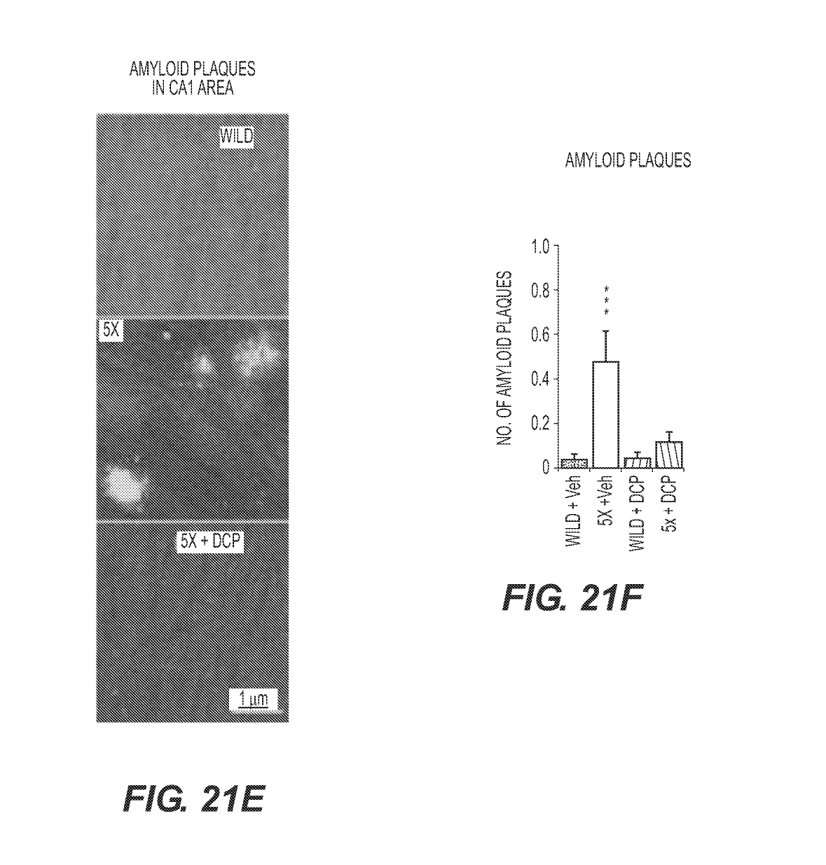

PKC activators in accordance with the present disclosure can reverse in vivo signs of neurodegeneration, such as protect against the losses of postsynaptic dendritic spines and synapses in the hippocampal area, and protect against the loss of presynaptic vesicles in Alzheimer's disease mice. (FIGS. 19 and 20). The PKC activators can also prevent learning and memory deficits and amyloid plaque formation in Alzheimer's disease mice. (FIG. 21).

Protocol: Bryostatin-1 (30 μg/kg, intraperitoneal injection) was administered to 2-month old Tg2576 mice twice a week. At five months old, hippocampal slices from the brains of the mice were processed for immunohistochemistry and confocal microscopy analysis. Results are shown for analysis of spinophilin density (A, B) not caused by neuronal loss (C, D). Bryostatin also prevented decreases in mushroom spine-shape dendritic spines (E-G), as evaluated with DiI staining and confocal microscopy; and synapses (H-J), as assayed with electron microscopy. Non-treated groups (wild-type and transgenic (Tg) mice) received the same vehicle volumes, mechanism of delivery, and frequency of administration as the treated groups.

Protocol: DCPLA (20 mg/m², tail vein injection) was administered to 2-month old 5XFAD mice twice a week. At five months old, hippocampal slices from the brains of the mice were processed for immunohistochemistry and confocal microscopy analysis. Results are shown for analysis of spinophilin density (A, B), synaptophysin density (A, D) not caused by axonal bouton (synaptophysin granules) (C) and neuronal loss (E, F). DCPLA also protected the decrease in mushroom spine shape dendritic spines and synapses (G, I). Non-treated groups (wild-type and transgenic (Tg) mice) received the same vehicle volumes, mechanism of delivery, and frequency of administration as the treated groups.

Protocol: 5-month old 5XFAD mice were trained for 5-6 days (3-4 trials/day) to find a hidden platform (9 cm diameter), submerged about 2 cm below the water surface in the a maze pool with 114 cm diameter. After the training trials, a probe trial (a quadrant test or memory retention trial) was given with the platform removed to assess memory retention for its location by the distance the mouse moved in the quadrants. Treatment (started at 2 months old, 2 times/week) of bryostatin (30 μg/kg, intraperitoneal injection) or DCP-LA (20 mg/m2, tail vein injection) prevented learning (A, C) and memory deficits (B, D), and reduced amyloid deposition (E, F). Non-treated groups (wild-type and transgenic (5×) mice) received the same vehicle volumes, mechanism of delivery, and frequency of administration as the treated groups

Stroke:

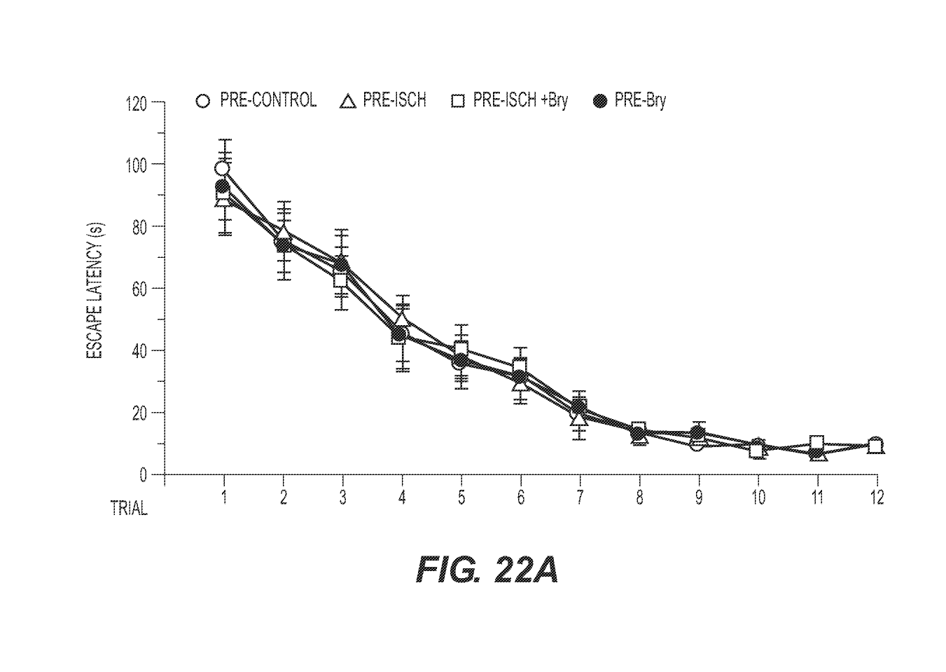

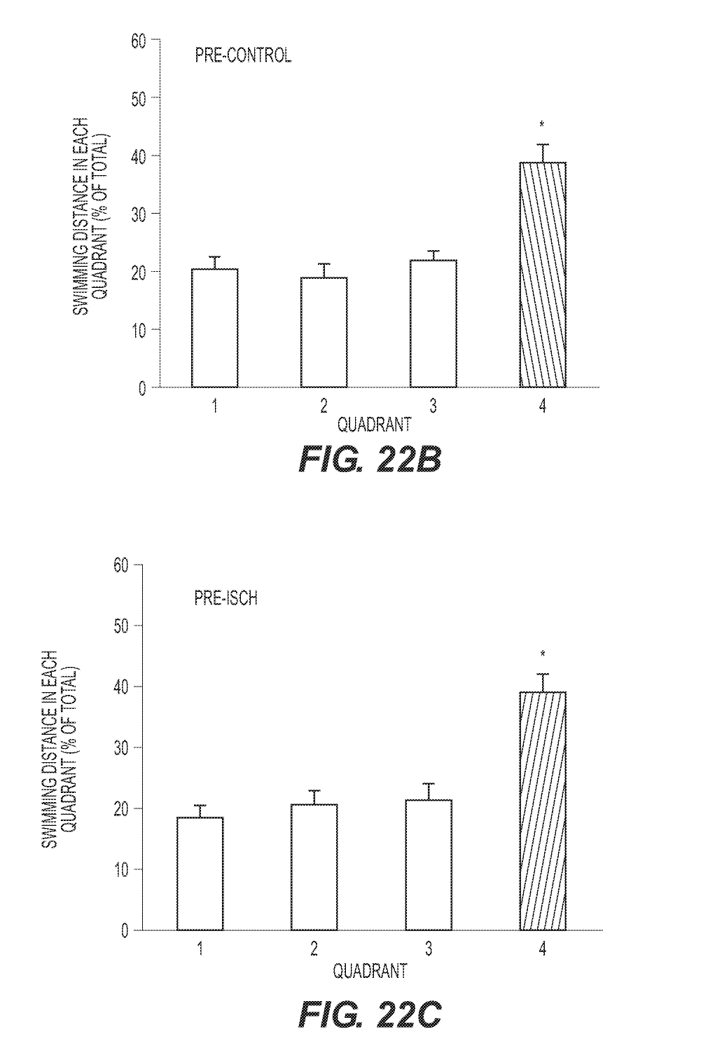

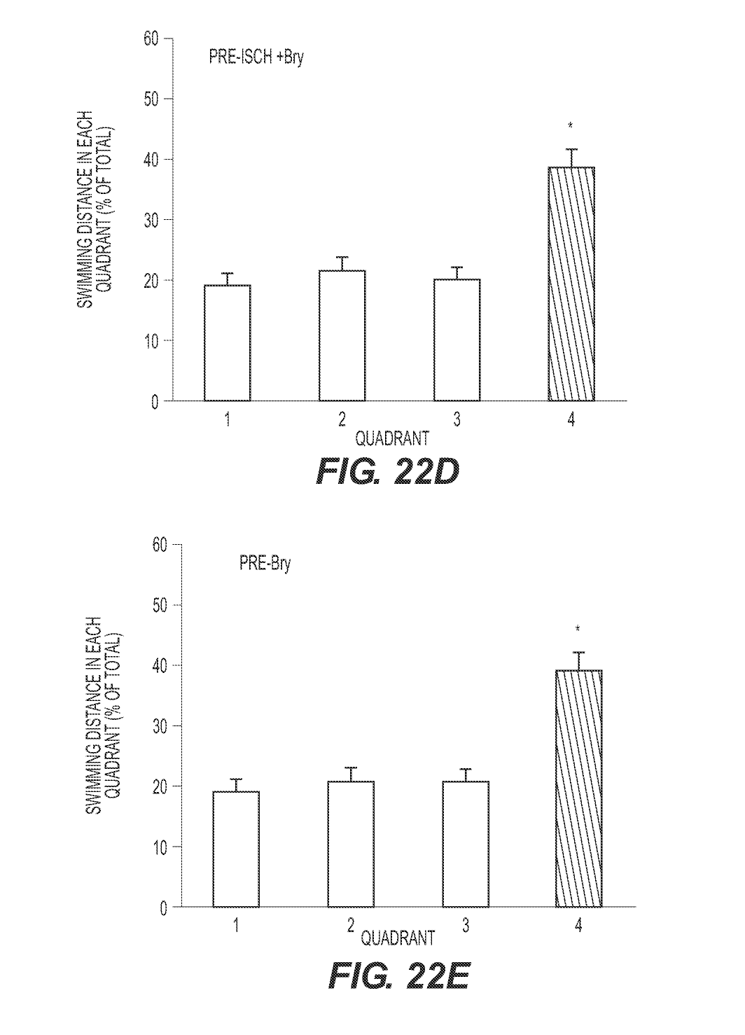

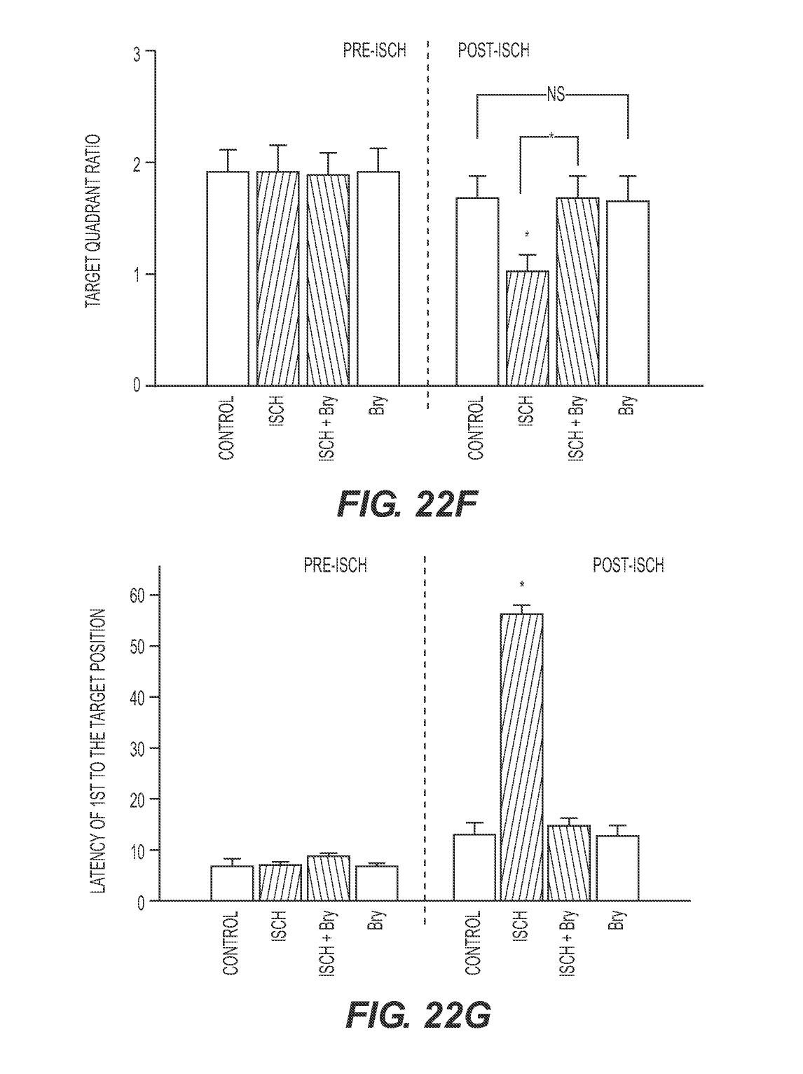

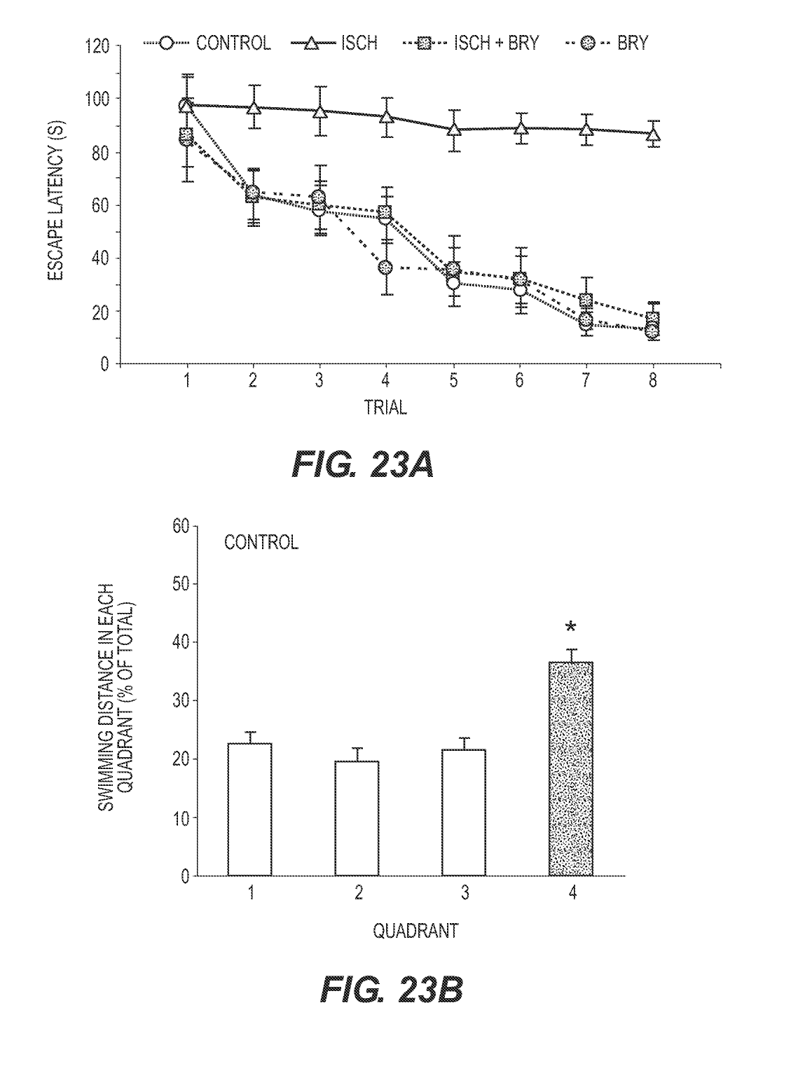

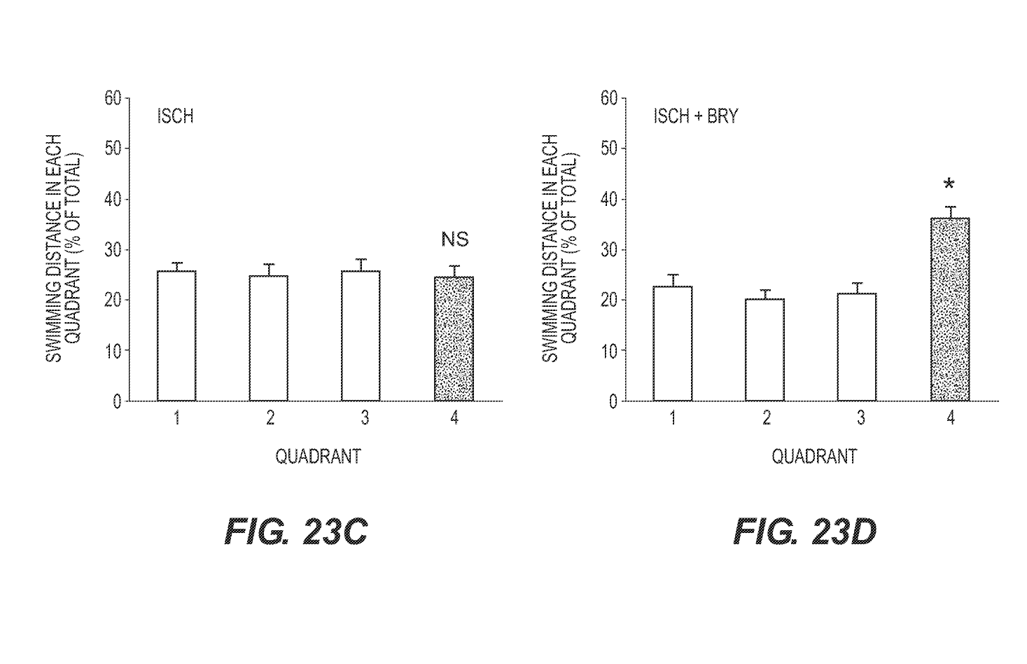

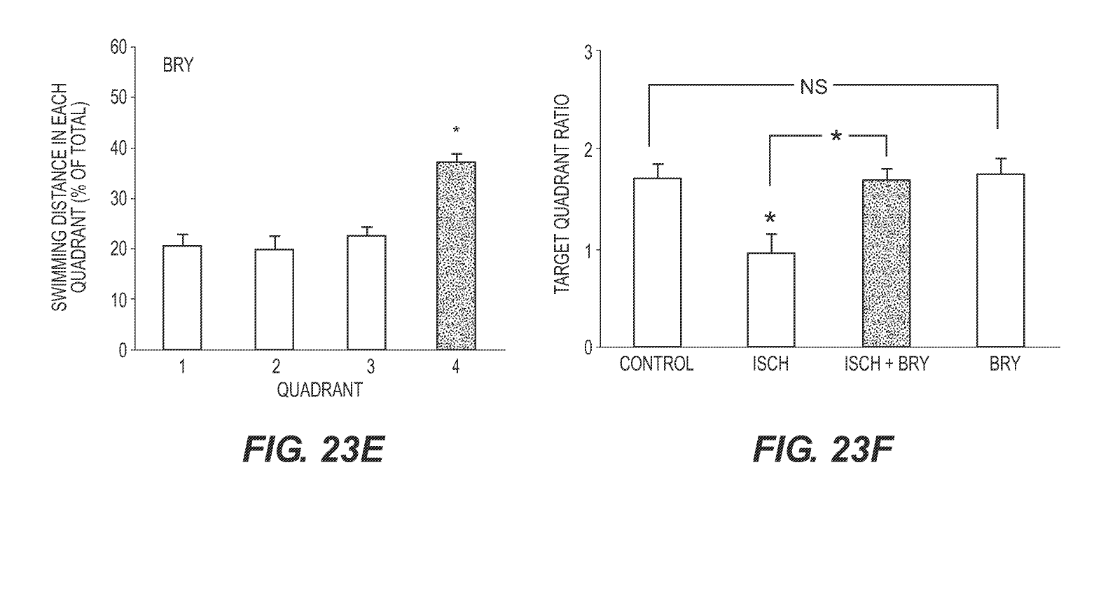

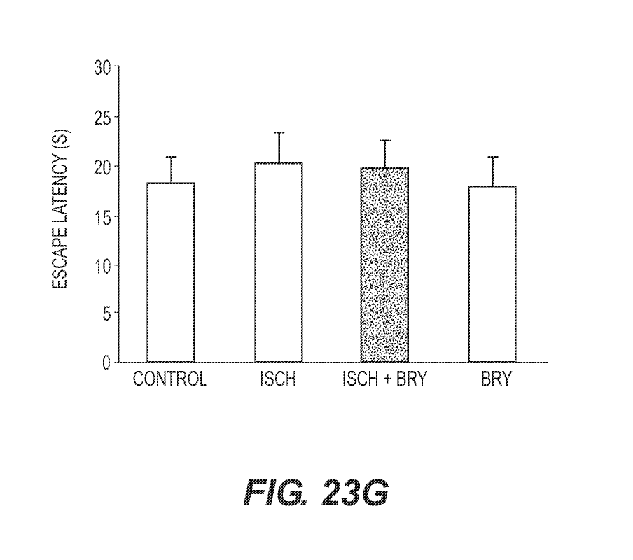

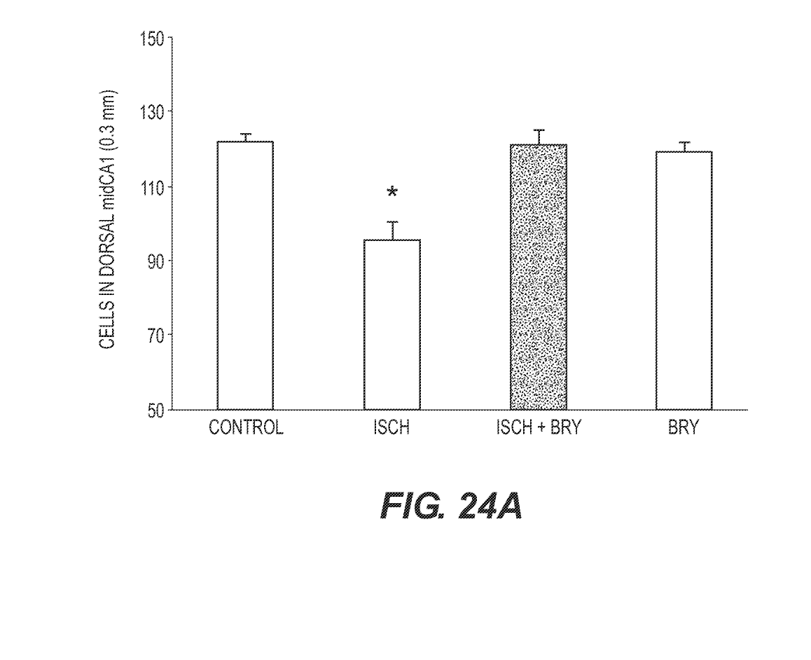

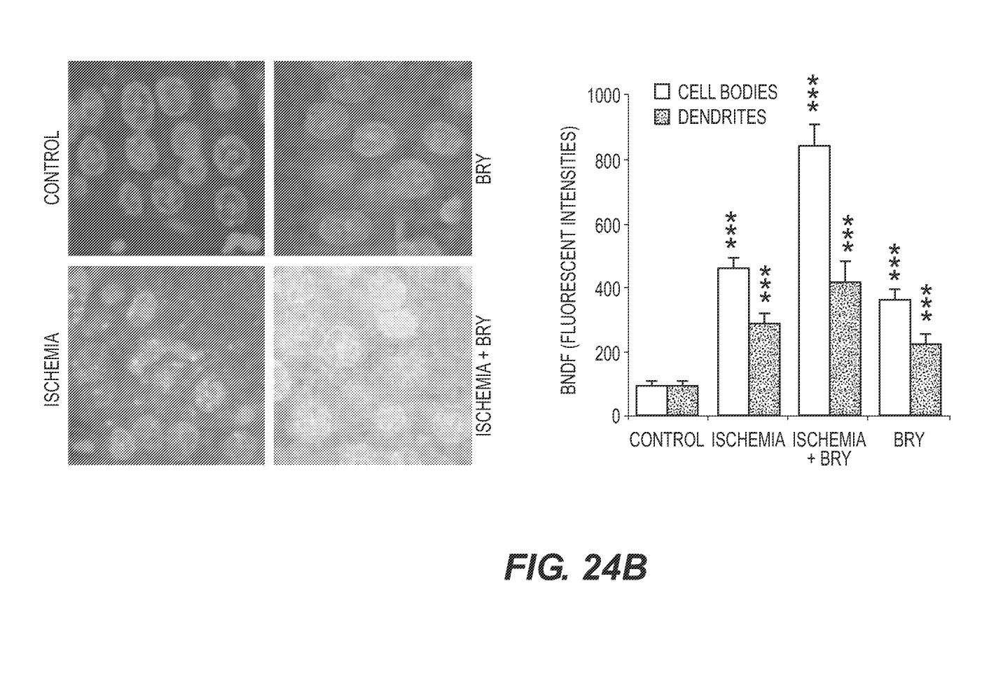

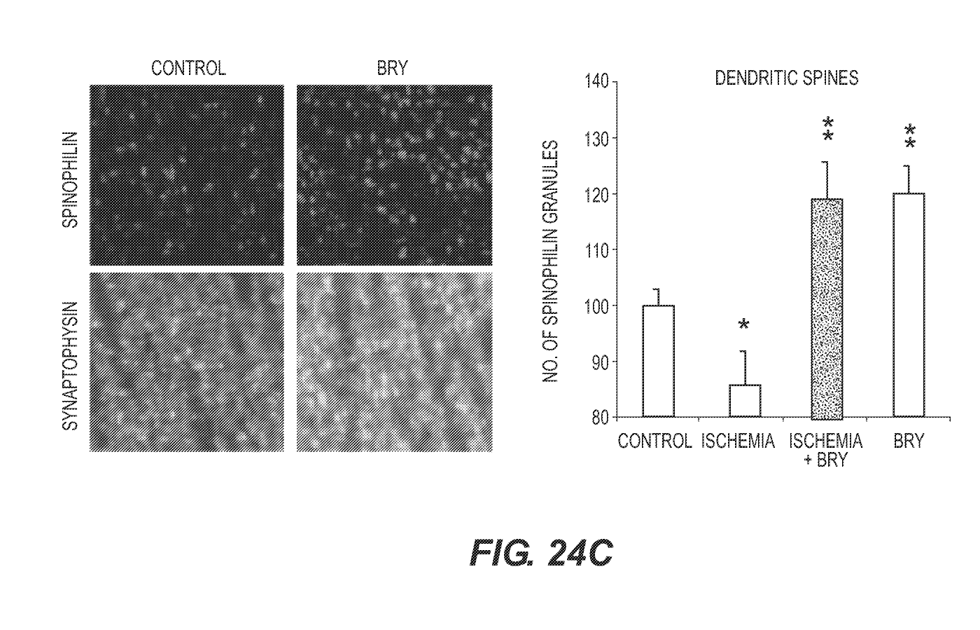

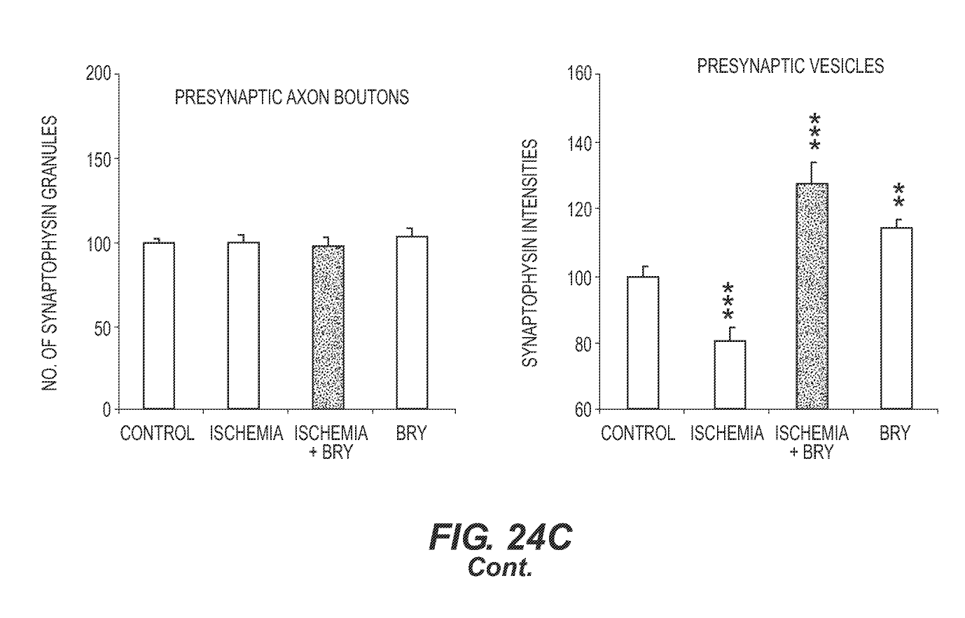

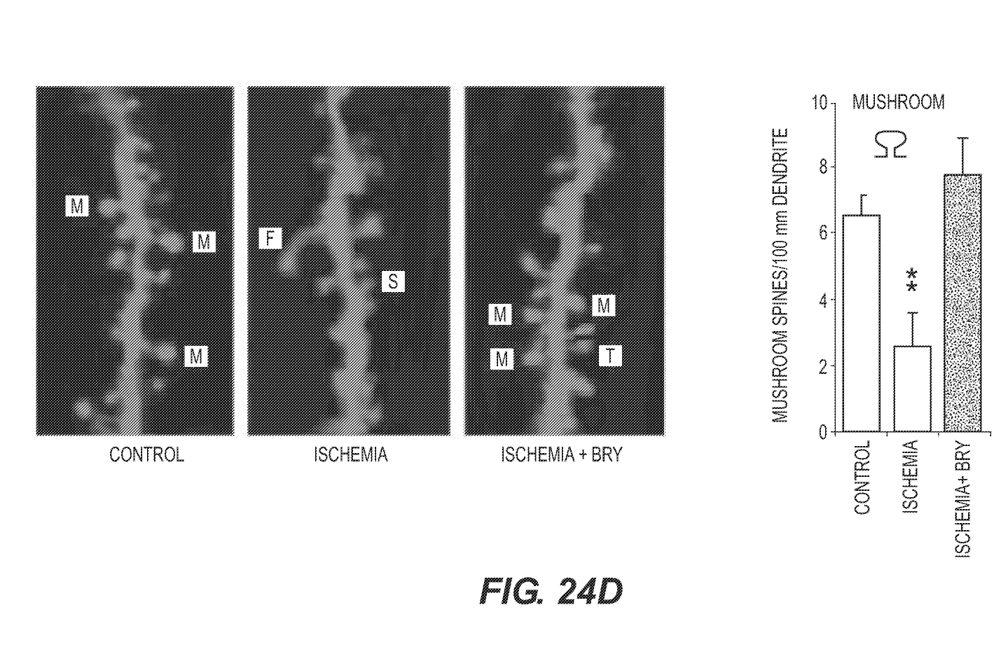

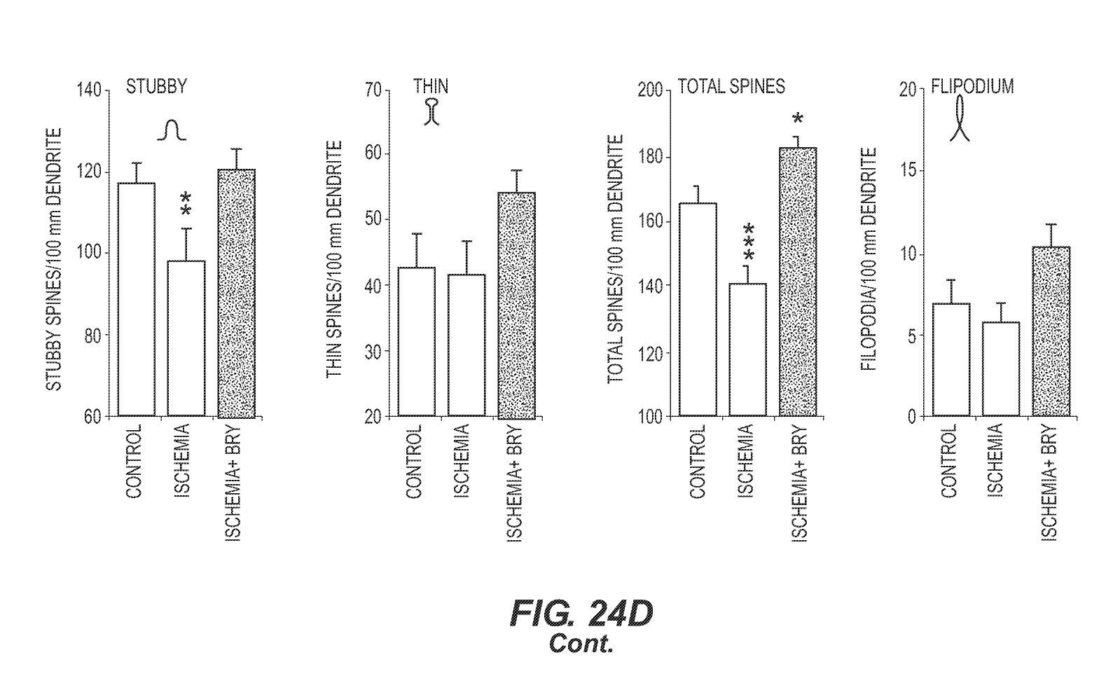

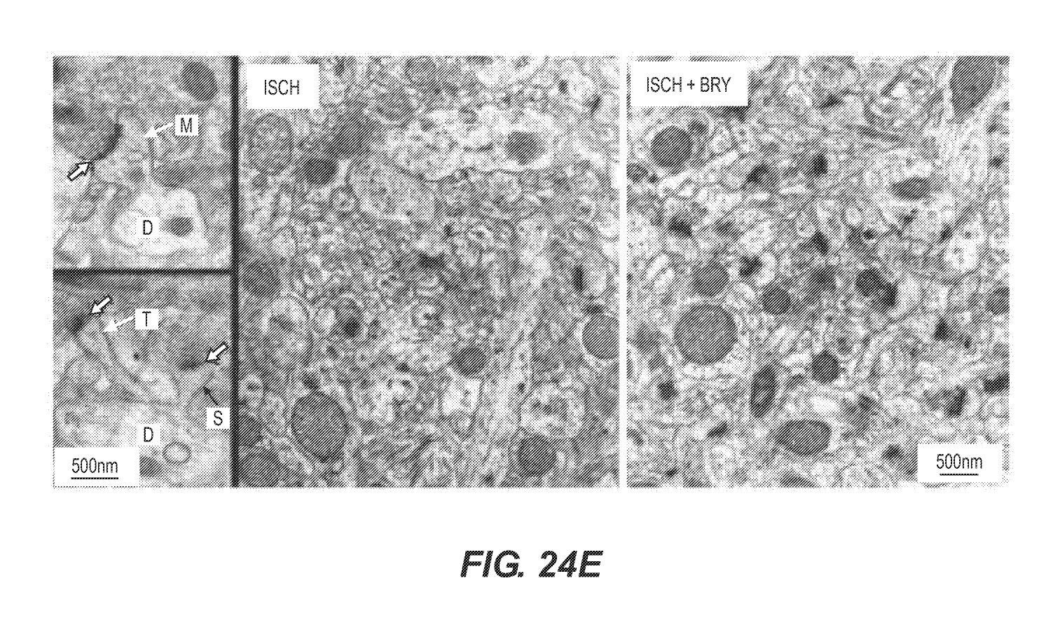

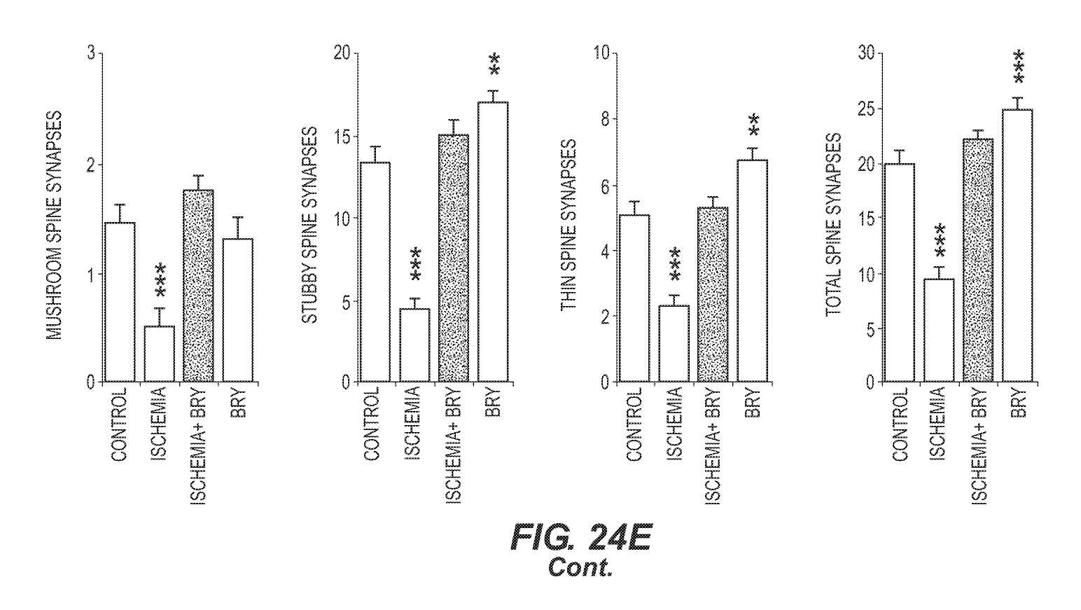

PKC activators in accordance with the present disclosure can reverse in vivo signs of neurodegeneration, such as rescue learning and memory loss associated with cerebral ischemia (FIGS. 22 and 23). The PKC activators can also prevent neuronal loss, increase neurotrophic activity and synaptic strength in the dorsal hippocampal CA1 area after cerebral ischemia-induced damage. (FIG. 24).

Protocol: An initial evaluation of the test rats was undertaken to observe their spatial learning (2 trials/day for 6 days) and memory (a probe test of 1 min, 24 hours after the last trial). Cerebral ischemia was induced 1 day after the probe test, followed by bryostatin-1 (15 μg/m²) administration through a tail vein (2 doses/week, for 10 doses), starting 24 hours after the end of the ischemic (2-VO)/hypoxic event. A second probe test was performed 2 weeks after the last bryostatin-1 dose. Results are shown in A for the spatial water maze performance of the rats over training trials (2 trials/day for 6 days) before the ischemia/treatment (means±SEM.; trials: F11,383=40.483, P<0.001; groups: F3,383=0.315, P>0.05). Results of the probe test after the training trials before the ischemia and/or treatment are shown in B-E (Quadrant 4 was the target quadrant). Results are shown for the target quadrant ratios before (pre-Isch) and after (post-Isch) the ischemia and/or treatment in F. Results are shown in G for the latency of the first crossing the target location before (pre-Isch) and after (post-Isch) the ischemia and/or treatment. There were eight rats/group (Bry—bryostatin-1; Isch.—cerebral ischemia) (*, P<0.05. NS: P>0.05).

Protocol: Bryostatin-1 (15 μg/m2) was administered through a tail vein (2 doses/week, for 10 doses), starting 24 hours after the end of the ischemic (2-VO)/hypoxic event. The ability of the rats in spatial learning (2 trials/day for 4 days) and memory (a probe test of 1 min, 24 hours after the last trial) was evaluated, with the first training started 9 days after the last dose of bryostatin-1. Results are shown in A for escape latency over training trials (mean±standard error of the mean), B-E depict results of the memory retention test after the training trials (Quadrant 4 was the target quadrant where the hidden platform was placed during the training trials), F shows results for the target quadrant ratio (calculated by dividing the target quadrant swim distance by the average swim distance in the non-target quadrants), and G shows results in a visible platform test (with a visible platform placed at a new location). (Bry—bryostatin-1; Isch—cerebral ischemia; NS—not significant) (*, P<0.05).

Protocol: Rats were administered bryostatin-1 (15 μg/m², tail vein injection) for 5 weeks beginning 24 hours after the end of the ischemic/hypoxic event. After 9 days after the last bryostatin-1 dose (approximately 7 weeks after the ischemic/hypoxic event), results indicate that bryostatin prevented neuronal loss (A). Bryostatin-1 also induced an increase in the immunofluorescence intensity of brain-derived neurotrophic factor (BDNF) induced by cerebral ischemia (B). Bryostatin-1 also protected the loss of dendritic spines and synapses, as shown in the confocal microscopy images depicted at C (immunohistochemistry), D (Dil staining of and with) E (electron microscopy). Non-treated groups received the same vehicle volumes, mechanism of delivery, and frequency of administration as the treated groups.

Traumatic Brain Injury:

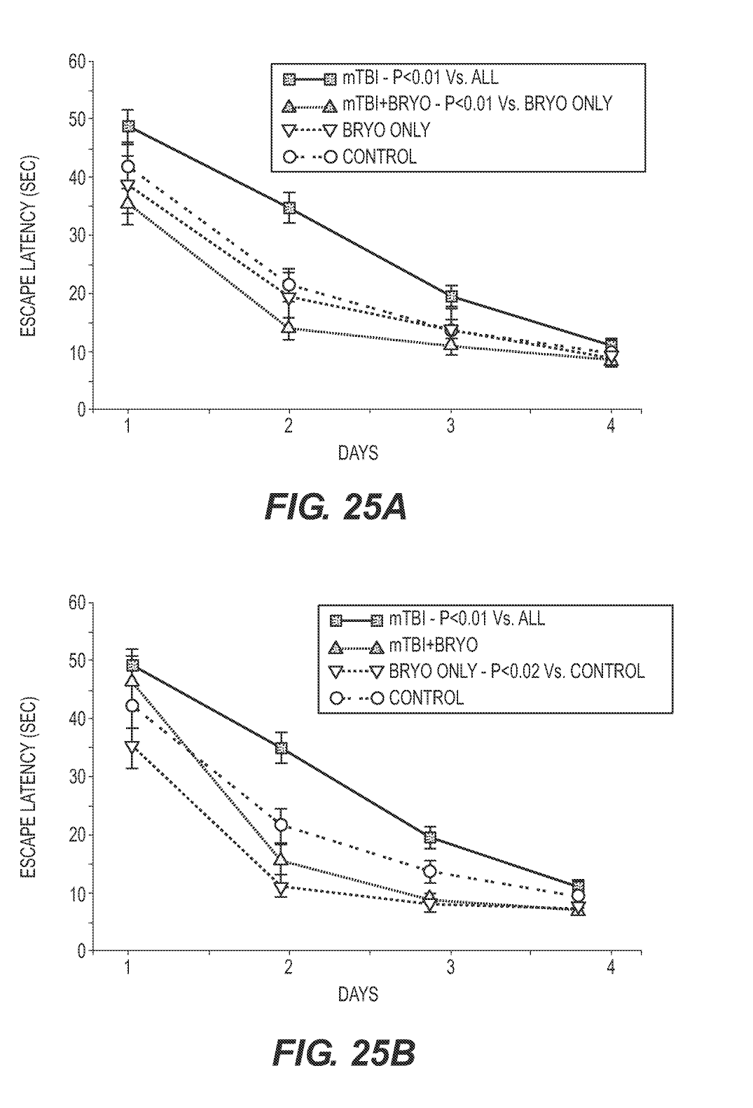

PKC activators in accordance with the present disclosure can reverse in vivo signs of neurodegeneration, such as protect against traumatic brain injury-induced cognitive deficits (FIG. 25).

Protocol: One hour after the minimal traumatic brain injury was induced, the mice received a 5×i.p. bryostatin-1 injection treatment over a period of 14 days, in two injection doses 20 and 30 μg/kg (N=9 in each group). One hour after the last injection of the series, the cognitive ability of the mice was tested in the MWM. Mice were tested for 4 days 6 times a day. On day 5 the platform was removed and the mice memory retention was tested. The results indicate that both doses completely protects against mTBI induced cognitive deficits. Data was analyzed using repeated measure one way ANOVA and presented as mean±S.E.M. Both doses protected the learning abilities of the injured mice (pb0.01). Repeated injections of both doses used here (20 and 30 μg/kg) protected against the mTBI induced learning deficits (pb0.01 and pb0.02 accordingly). The higher injections dose (30 μg/kg—“C”) had also improved the learning of control uninjured mice (pb0.02), while the lower dose (20 μg/kg—“A”) had no effect on uninjured mice. The lower dose administered to the mTBI group improved their acquisition of the learning task even beyond that of control mice (pb0.015).

Mental Retardation:

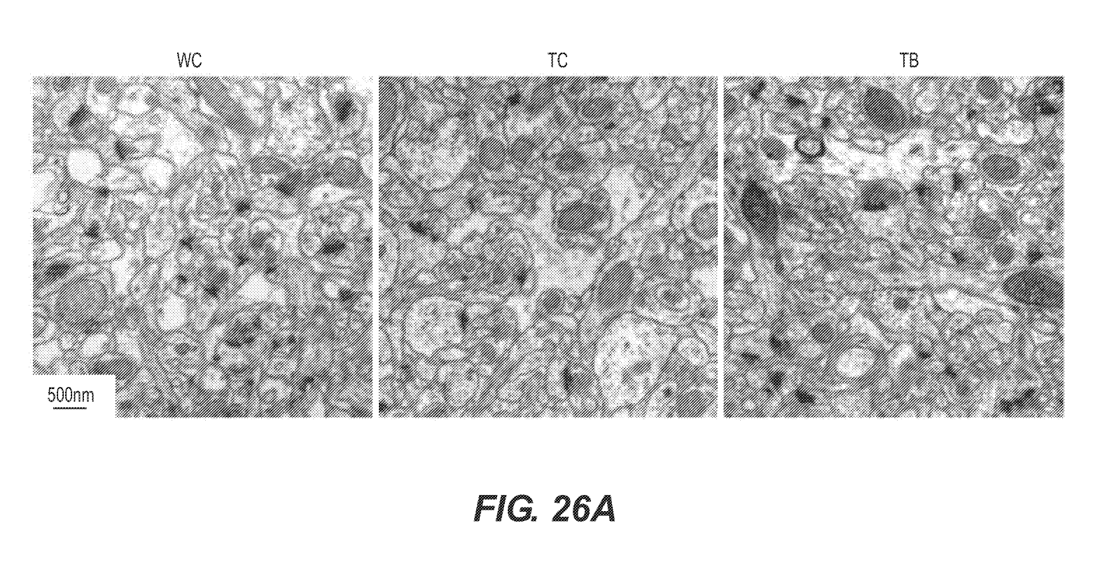

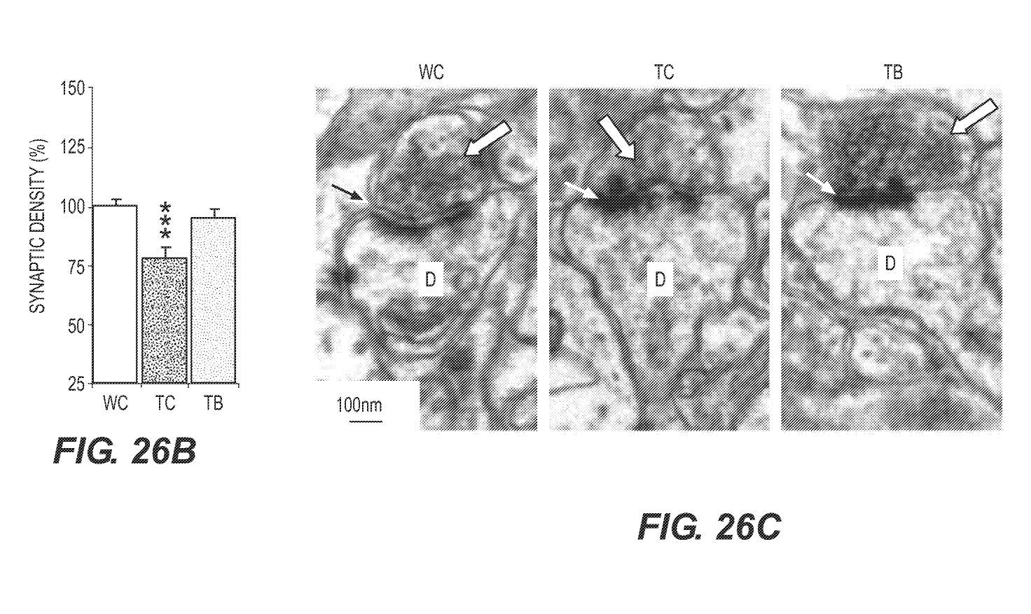

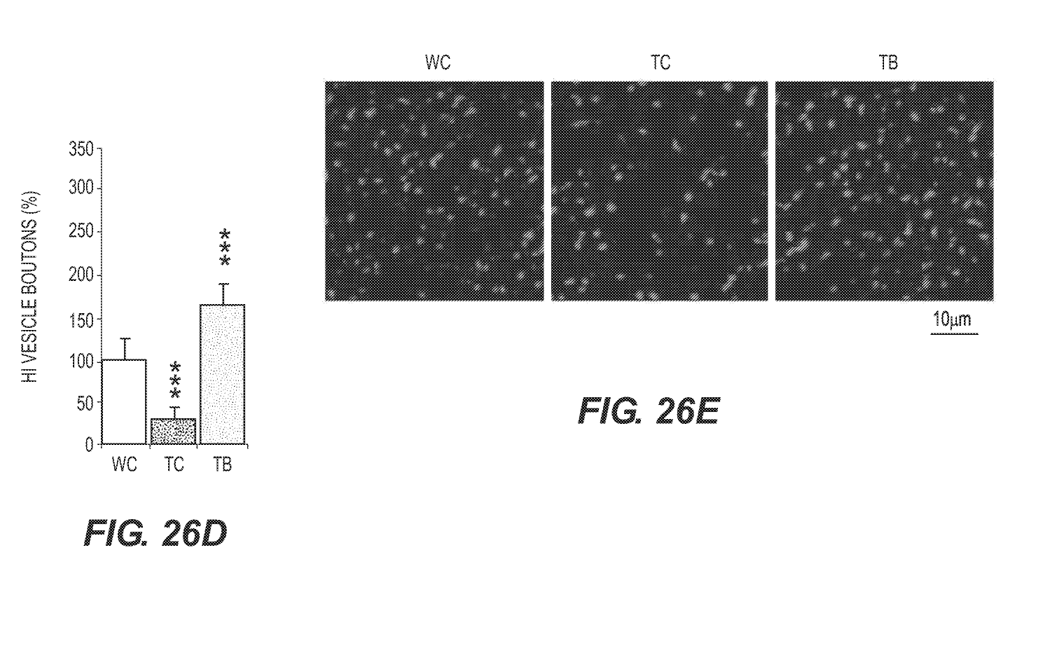

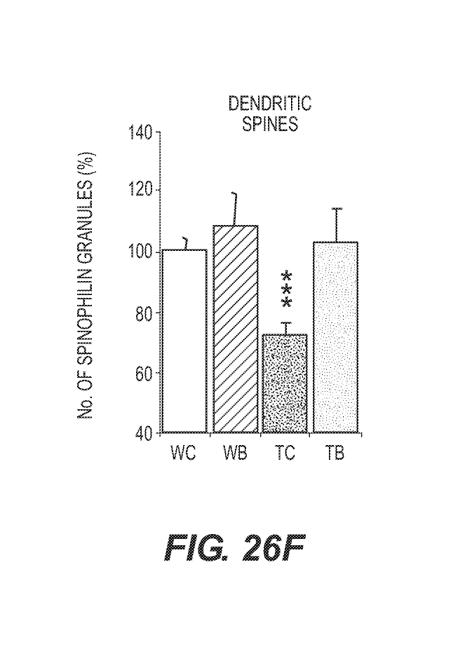

PKC activators in accordance with the present disclosure can reverse in vivo signs of neurodegeneration, such as restoring the number of synapses in fragile X transgenic mice (FIG. 26).

Protocol: Bryostatin (25 μg/kg body weight, intraperitoneal injection) was administered to 2 month old fragile X transgenic mice twice a week for 3 months. The results show that bryostatin rescued the losses of synapses (A, B), presynaptic vesicles within presynaptic axonal boutons (C, D), and postsynaptic dendritic spines (E, F). Non-treated groups (WC and TC) received the same vehicle volumes, mechanism of delivery, and frequency of administration as the treated groups.

Enhancement of Learning and Memory in Normal Animal Models

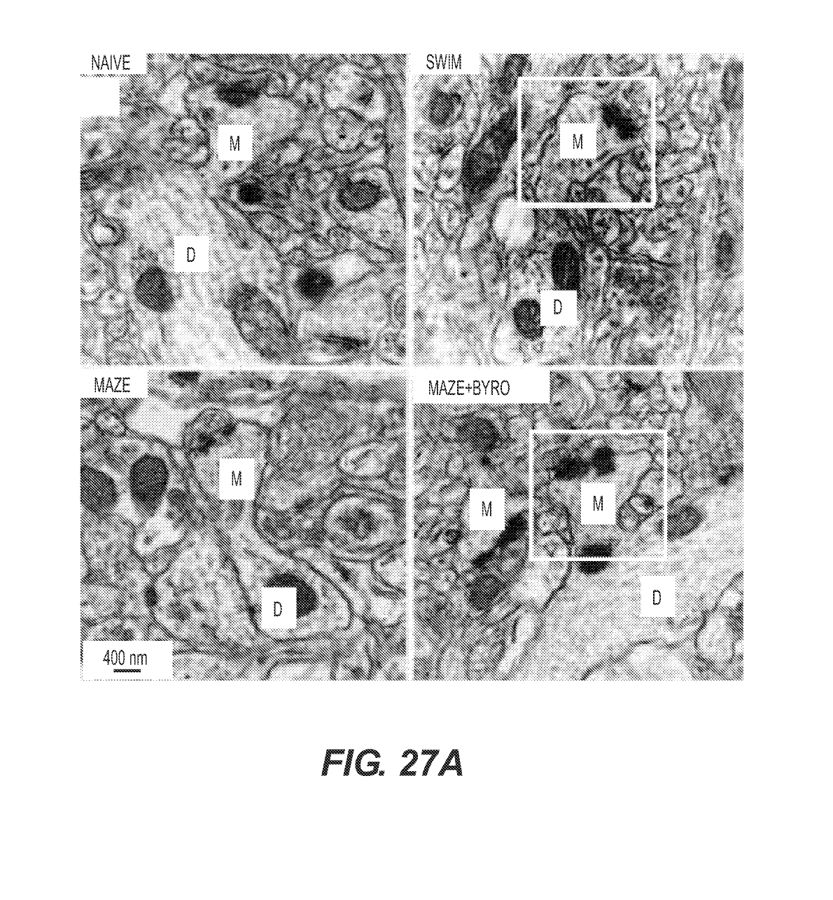

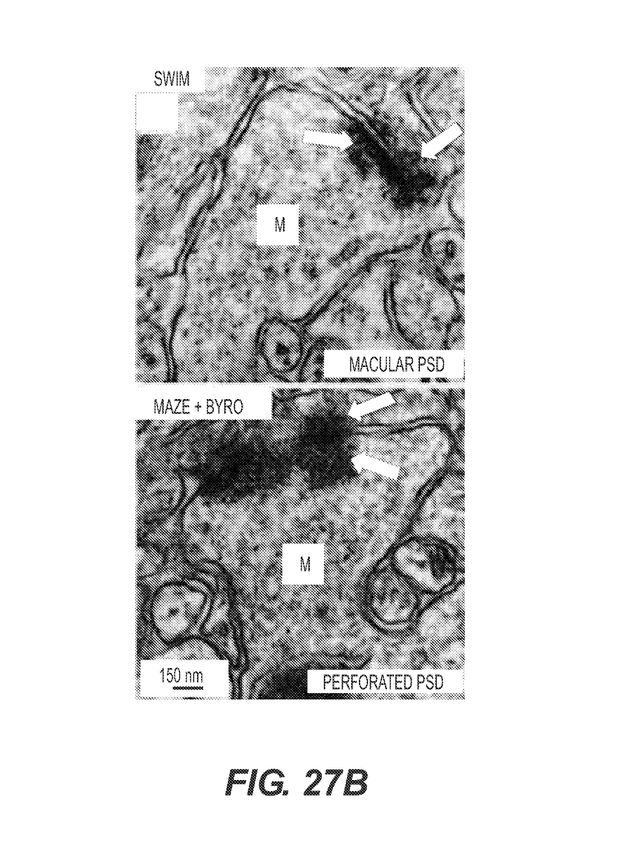

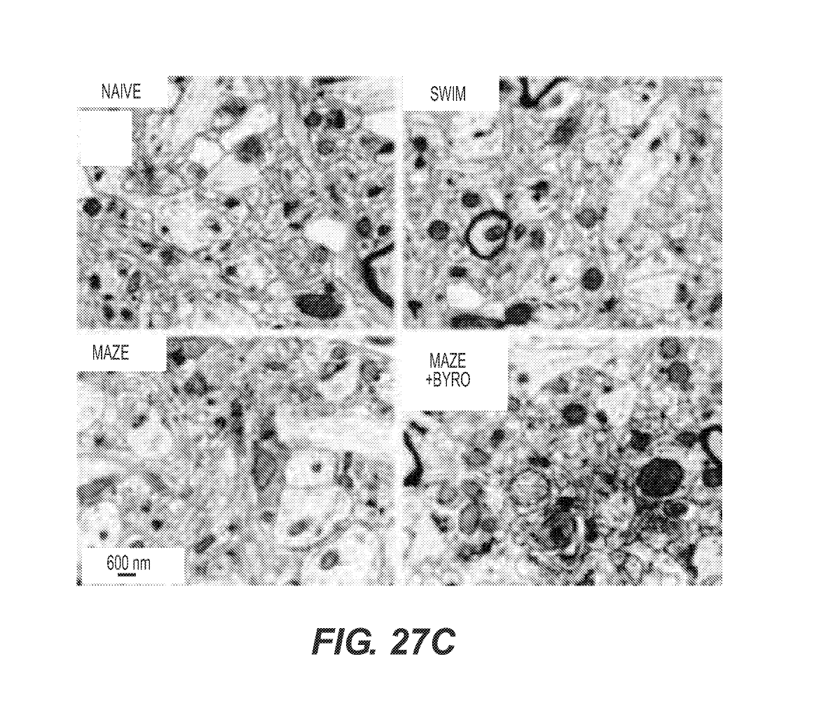

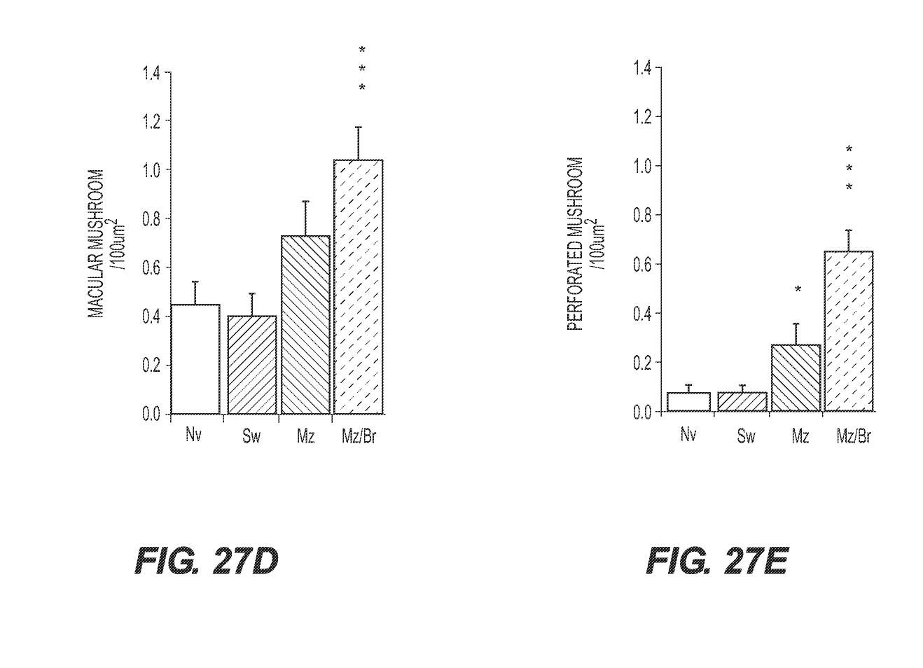

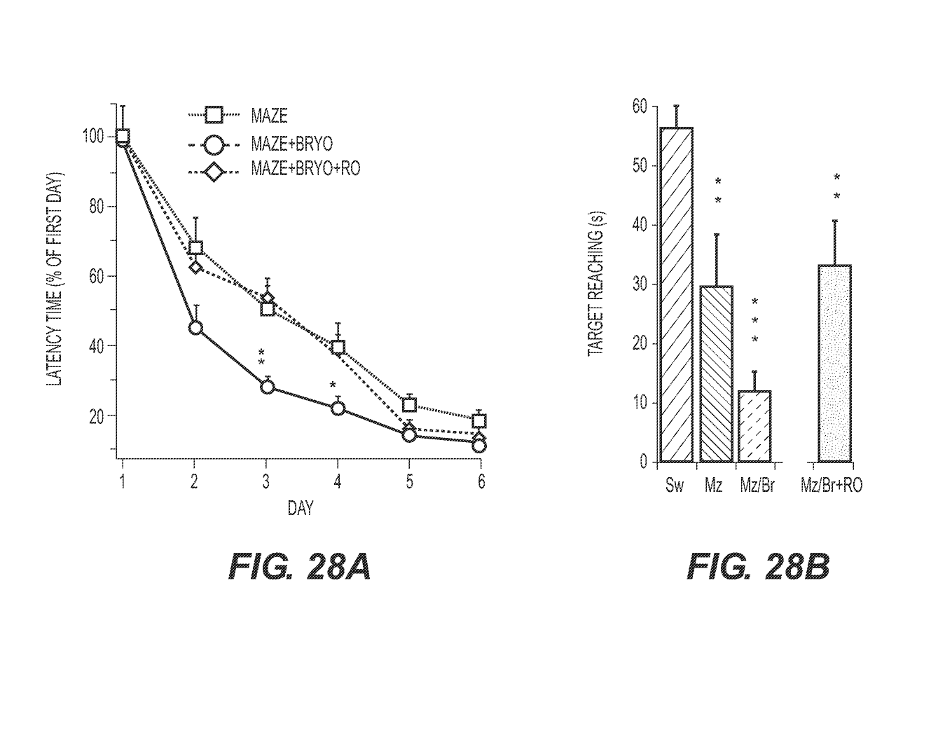

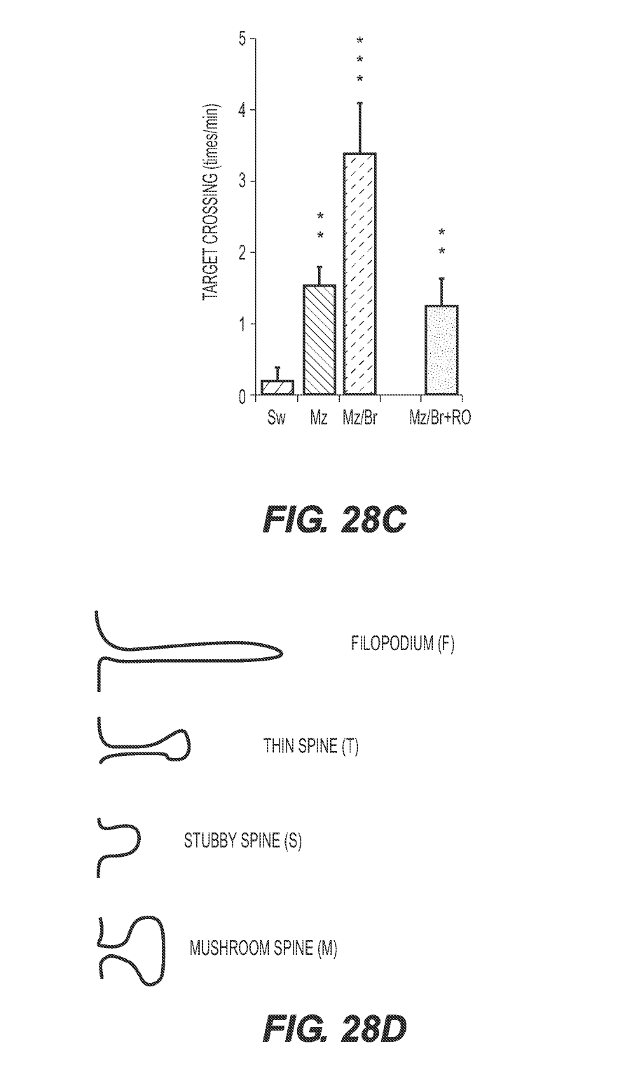

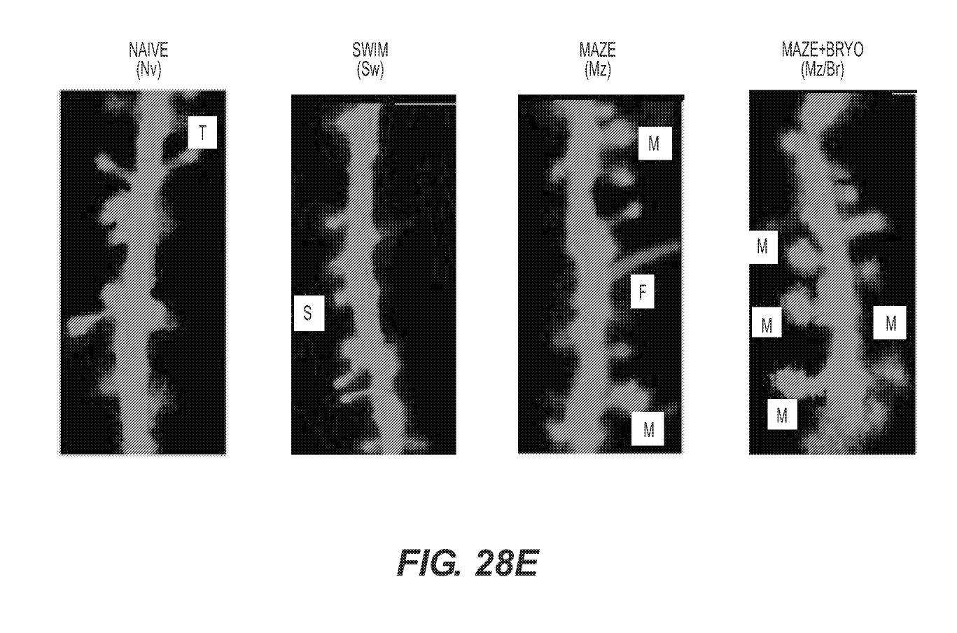

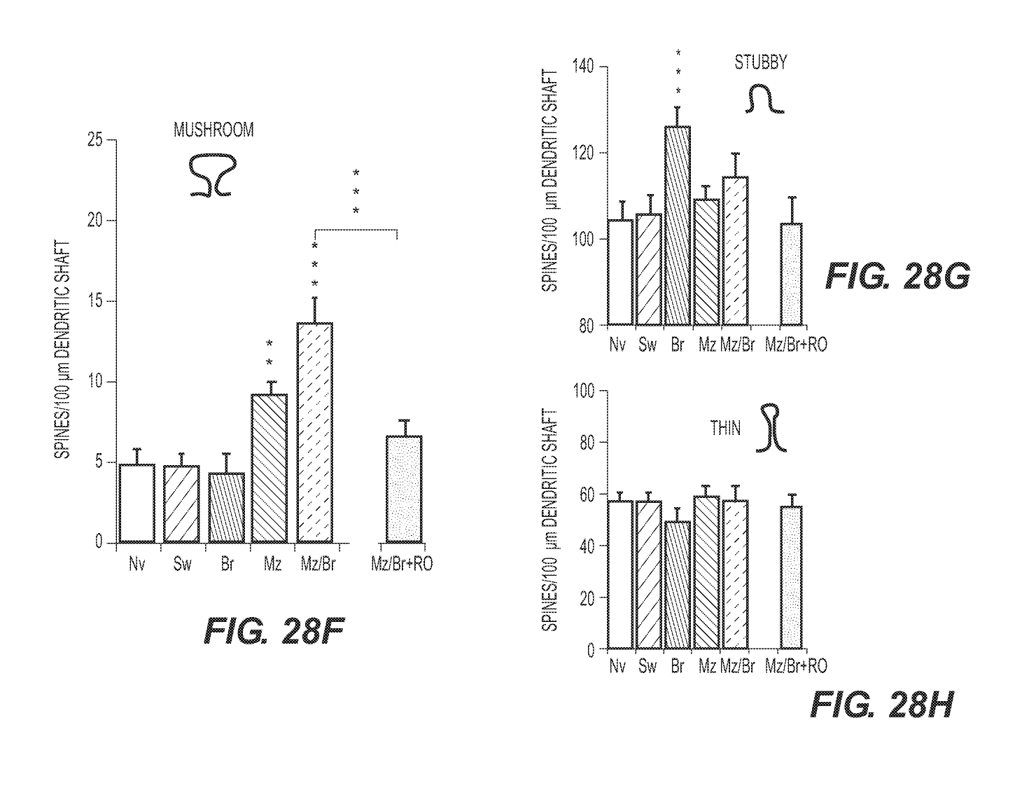

PKC activators in accordance with the present disclosure can enhance mushroom spine formation and synapses associated with learning and memory in healthy rats after water maze training (FIGS. 27 and 28).

Protocol: Non-diseased, healthy brown Norway rats (at 4-5 months old) were used in this study. Bryostatin enhanced the formation of mushroom spines in healthy rats after water maze training as shown in a-e. Memory retention after water maze training (4 swims per days for 5 days) increased the number of mushroom dendritic spine and synapses with (e) perforated postsynaptic densities (PSDs), but not with macular PSDs (d). Bryostatin given during water maze training significantly increased (d) mushroom spines with macular PSDs and enhanced (e) mushroom spines with perforated PSDs. Nv=naîve controls; Sw=swim controls; Mz=water maze treatment; and Mz/Br=water maze treatment plus bryostatin treatment.

Protocol: Non-disease, healthy brown Norway rats (at 4-5 months old) were used in this study. Bryostatin given during water maze training (10 mg/kg body weight, intraperitoneal injection, 3 doses every other days) promoted learning acquisition (a), memory retention (b, c), and promotes memory-specific induction of mushroom spine synapses that was inhibited with a PKCα inhibitor Ro 31-8220 (d-f). Bryostatin alone increased non-mushroom spine density in naïve rats (g). (Nv=naîve controls; Sw=swim controls; Mz=water maze treatment; and Mz/Br=water maze treatment plus bryostatin treatment).

Induction of Downstream Synaptogenic Biochemical Events

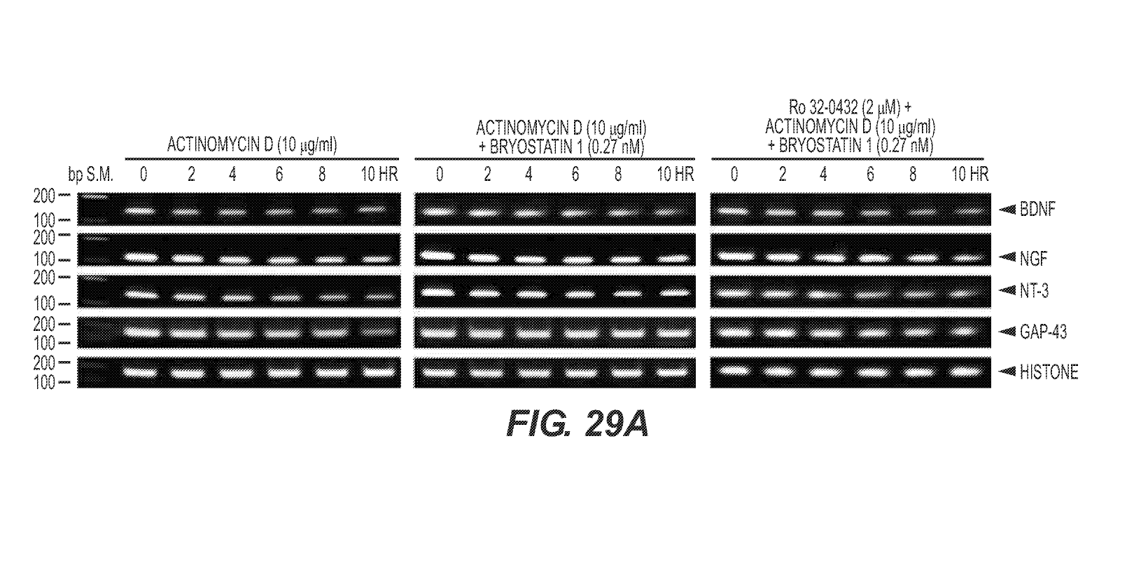

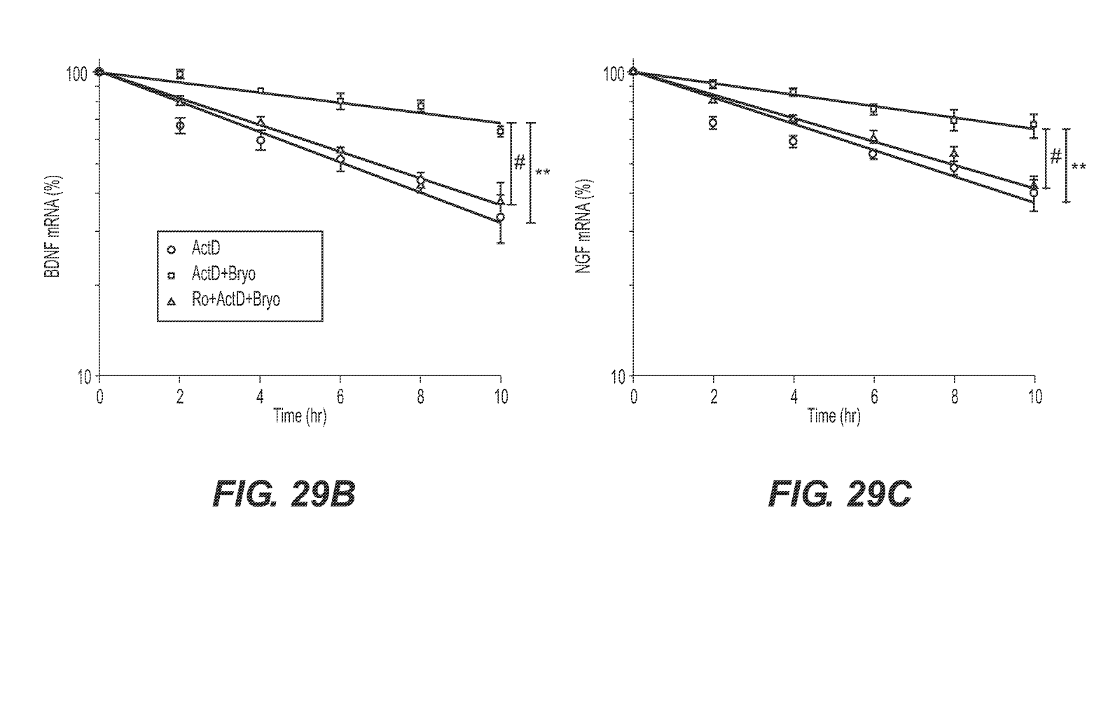

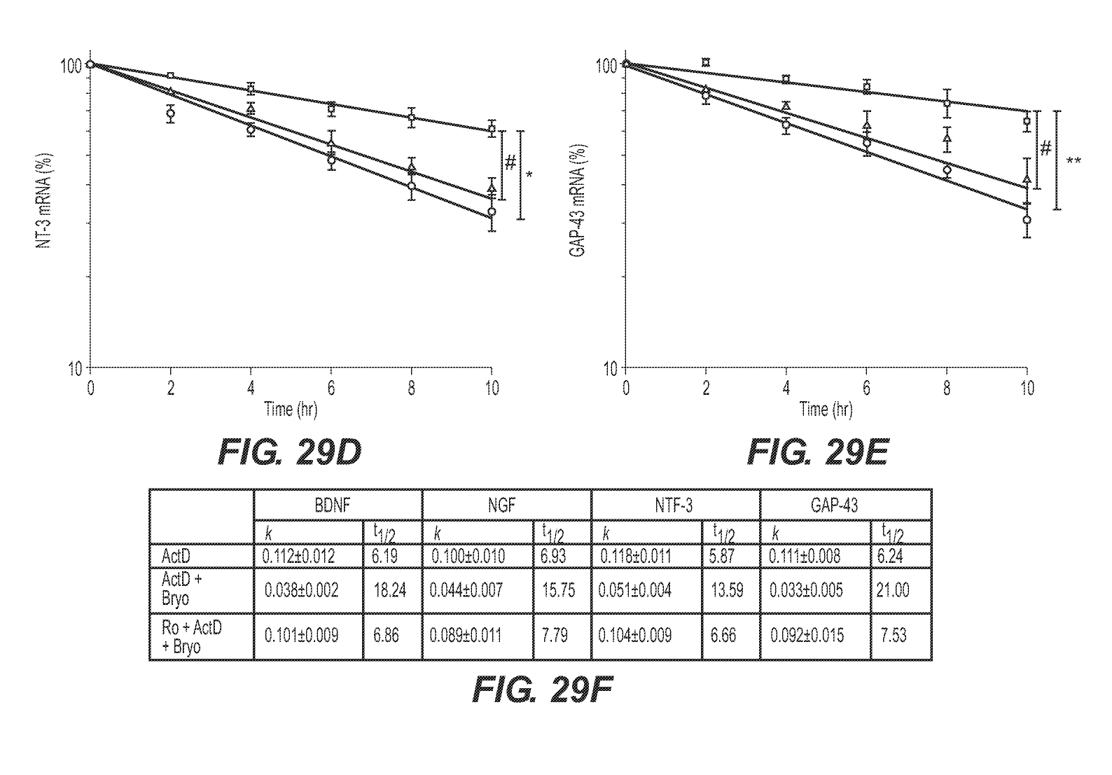

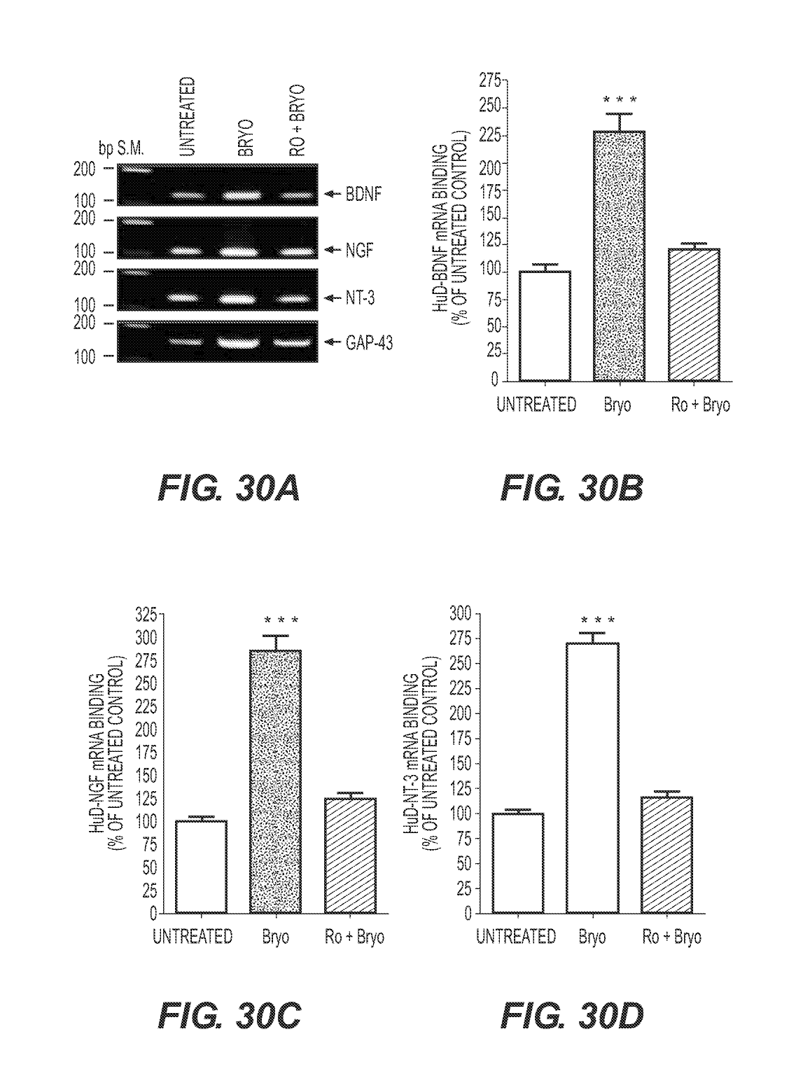

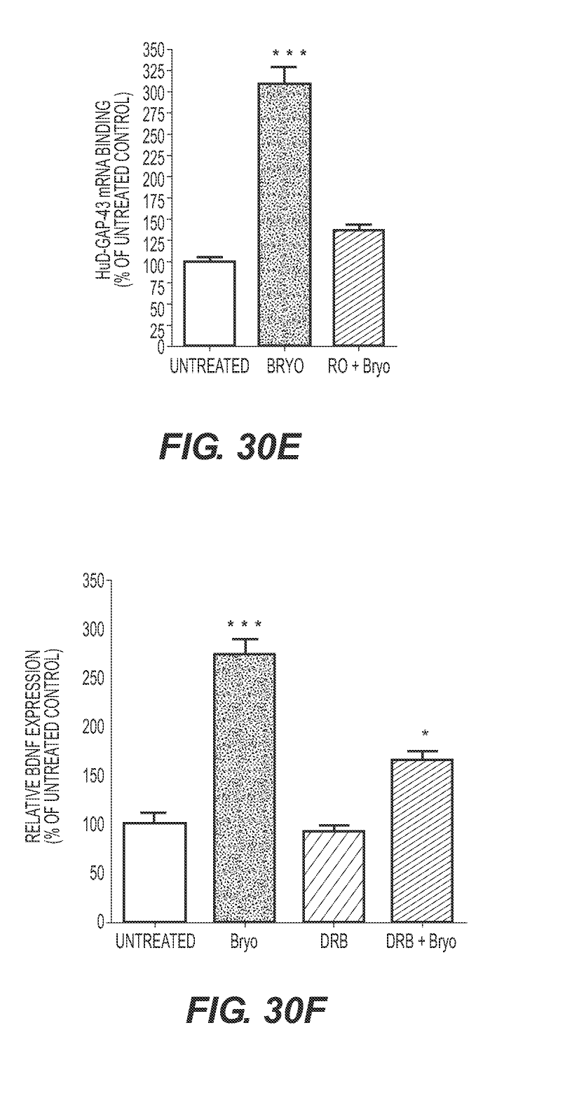

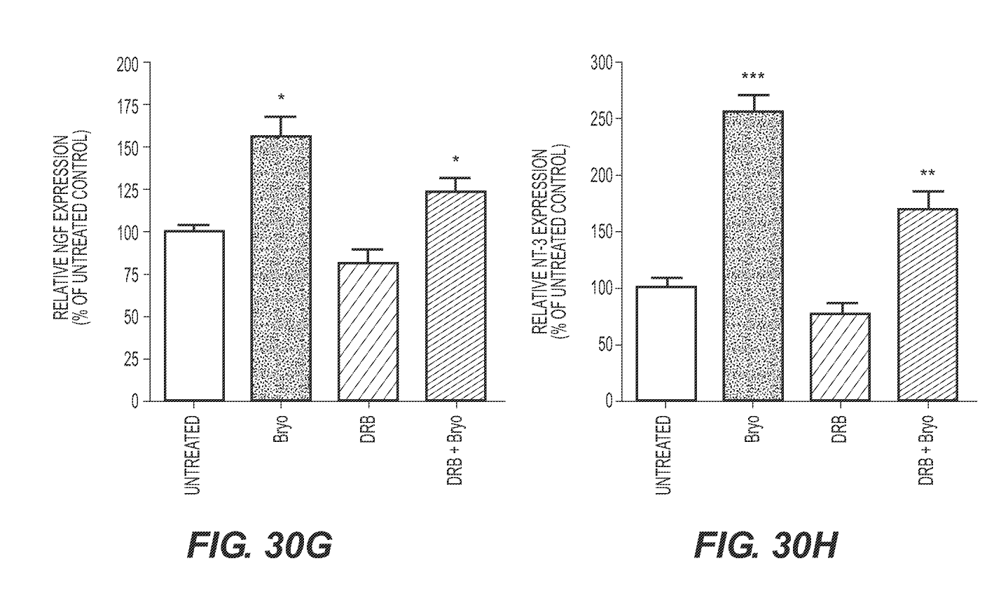

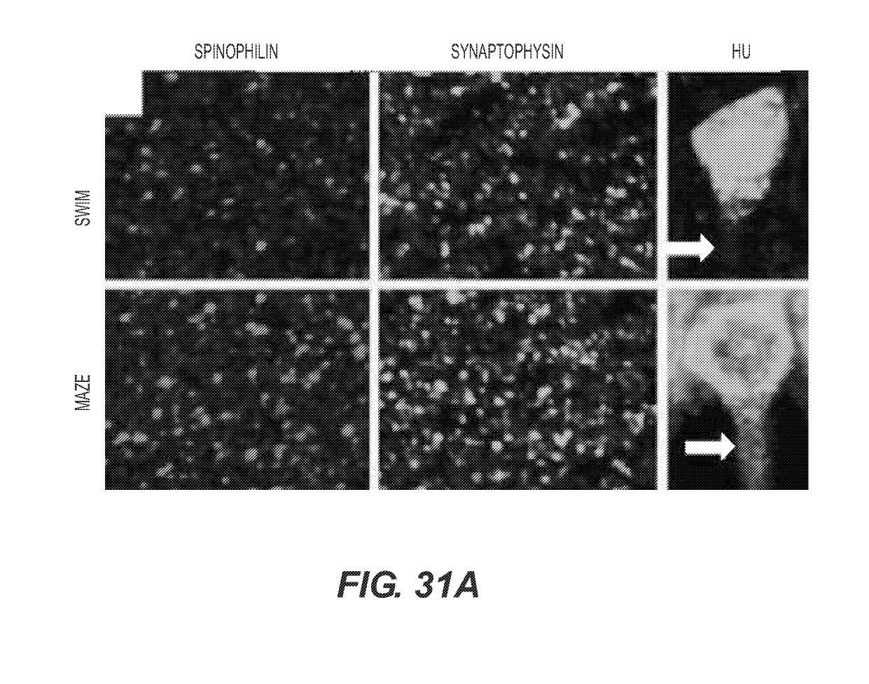

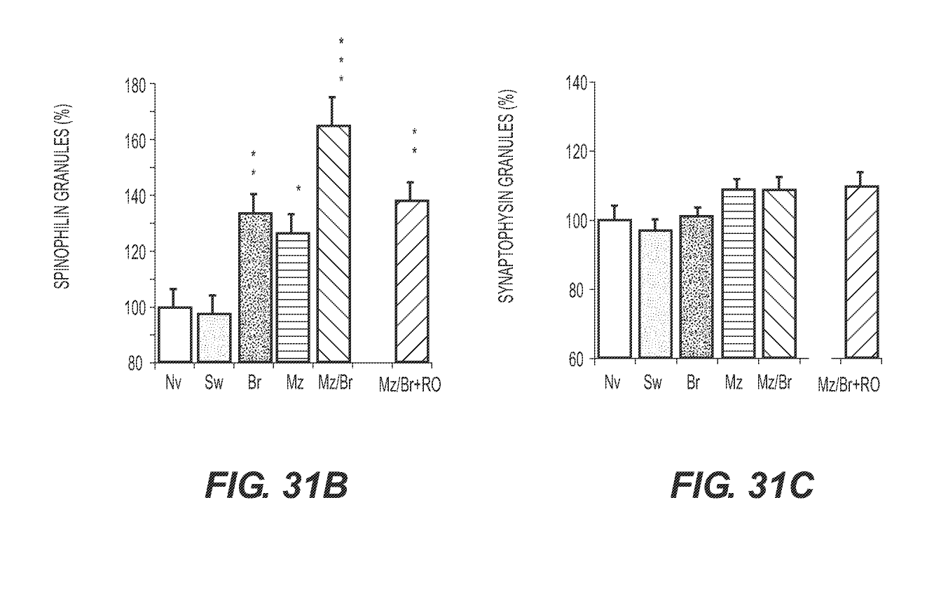

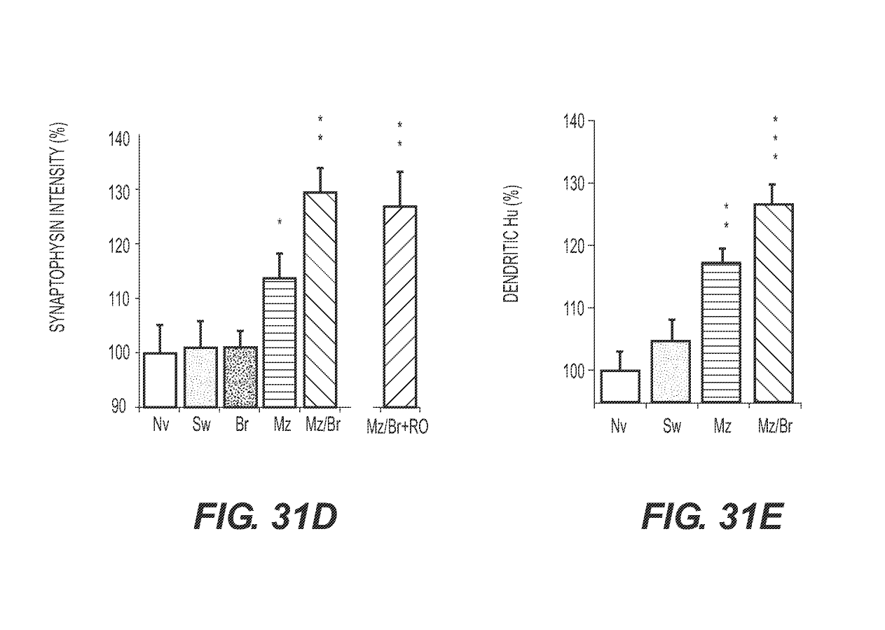

PKC activators in accordance with the present disclosure can induce downstream synaptogenic biochemical events such as enhance protein synthesis of neurotrophic factors (FIGS. 29-31).

Protocol: After primary rat hippocampal neurons were treated with actinomycin D (ActD; 10 mg/ml, a transcription inhibitor), ActD+bryostatin (0.27 nM), or pre-treated with Ro 32-0432 (Ro, 2 μM) for 2 hours and then treated with ActD+Bryostatin for 2, 4, 6, 8, and 10 hours, total RNA was isolated and used for quantitative RT-PCR using specific primers against BDNF, NGF, NT-3, GAP-43, or Histone mRNA as a control (A). Representative gels of RT-PCR from three independent experiments are shown in B-F. The content of NTFs mRNAs was quantified by real time RT-qPCR from neurons treated as in A. Each mRNA amount at each time point was compared with the initial mRNA level (100%). A nonlinear regression analysis was conducted, which gave a first-order decay constant (k). Average mRNA half-life (t_{1/2}) was calculated as 0.693/k and reported in the table F (Mean+SEM of three independent experiments, *P<0.05, **P<0.01, ActD+Bryostatin compared with ActD to assess the bryostatin effect; #P<0.05, ActD+Bryostatin compared with Ro+ActD+Bryostatin to assess the Ro 32-0432 effect).

Protocol: After primary hippocampal neurons were untreated or treated with bryostatin

(0.27 nM) or pre-treated with PKC inhibitor Ro 32-0432 (Ro, 2 μM) for 2 hours and then treated with bryostatin for 1 hour, cells were lysed and used for immunoprecipitation using HuD protein antibody. From immunoprecipitates, total RNA was isolated and used for RT-PCR to amplify BDNF, NGF, NT-3, and GAP-43 mRNAs associated with HuD proteins. A representative gel for RT-PCR data is shown from three different experiments. Relative amounts of (B) BDNF, (C) NGF, (D) NT-3, and (E) GAP-43 mRNAs bound to HuD are shown as % change (Mean+SEM, **P<0.01, ***P<0.001, compared with untreated control) were analyzed by real time RT-qPCR (A-E). After primary hippocampal neurons were untreated or treated with bryostatin (0.27 nM), 5,6-dichlorobenzimidazole-1 Dribofuranoside (DRB, 50 μM, a transcription inhibitor), or DRB for 1 hour prior to bryostatin for 6 hours, cells were lysed and then total amount of (F) BDNF, (G) NGF, or (H) NT-3 protein was quantitatively measured by ELISA and relative expression was presented as % change (F-H) (Mean+SEM, n>6 from three independent experiments, *P<0.05, **P<0.01, ***P<0.001, compared with untreated control).

Protocol: Non-diseased, healthy brown Norway rats (at 4-5 months old) were used in this study. Two days after 6-days of training, increases in dendritic spines (a, b) and presynaptic vesicle concentration (a, d) within unchanged axonal bouton density (a, c) were correlated with an increase in the nuclear export of HuC and HuD proteins into the dendritic shaft as compared with naive and swim controls (a, e). Those changes were enhanced with bryostatin treatment (10 μg/kg body weight, intraperitoneal injection, 3 doses every other day). Nv=naîve controls; Sw=swim controls; Mz=water maze treatment; and Mz/Br=water maze treatment plus bryostatin treatment).

Increases in Activity of A-β Degrading Enzymes

Neprilysin Activity

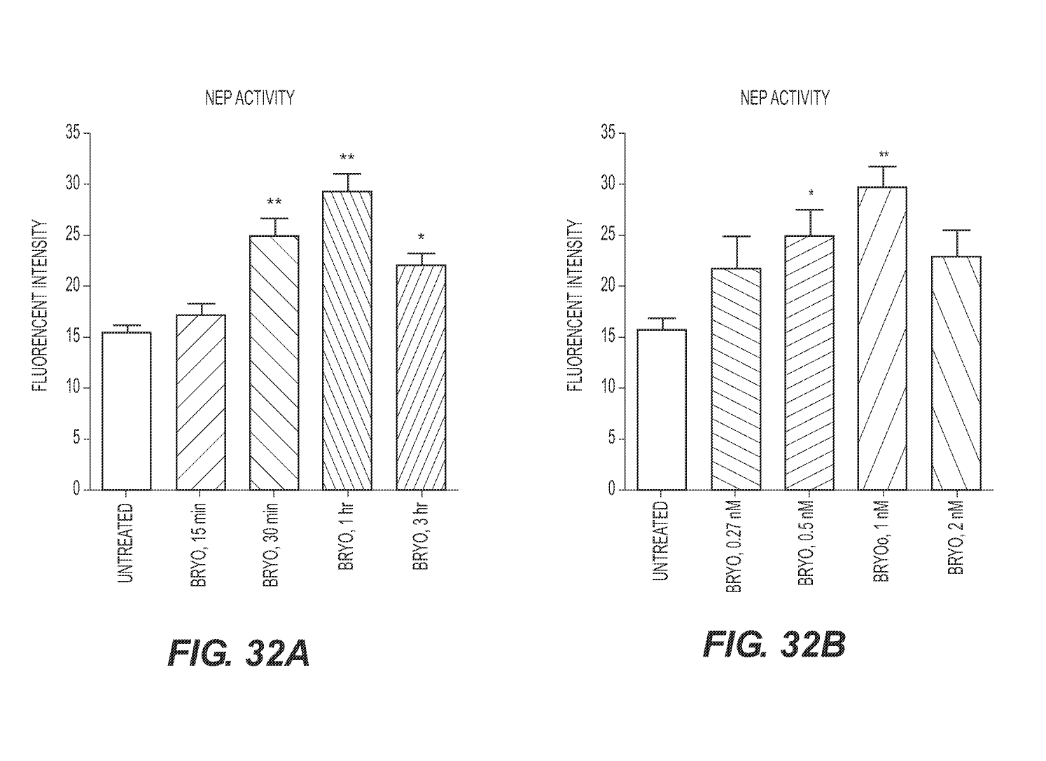

Protocol: Intact SH+hNEP cells were incubated in the absence or presence of bryostatin (1 nM) for 15 min, 30 min, 1 hour, or 3 hours. Cells were then lysed and neprilysin activity was measured. 50 μg of total lysates were separately incubated with 0.5 mM glutaryl-Ala-Ala-Phe-4-methoxy-2-naphthylamide as a substrate. Further incubation with leucine aminopeptidases released free 4-methoxy-2-naphthylamide that was measured fluorometrically at an emission of 425 nm (Mean+SEM of three independent experiments, *P<0.05; **P<0.01, Bryo compared with untreated condition). Intact SH+hNEP cells were incubated in the absence or presence of bryostatin (Bryo) at 0.27, 0.5, 1, or 2 nM concentration for 1 hr, lysed and then neprilysin activity for each condition was fluorometically measured as shown in (A) (Mean+SEM of three independent experiments, *P<0.05; **P<0.01, Bryo compared with untreated condition). Results are shown below in FIG. 32 (A—fluorometric results of free 4-methoxy-2-naphthylamide measured at different time points; B—fluorometric results of free 4-methoxy-2-naphthylamide measured at different concentrations of bryostatin).

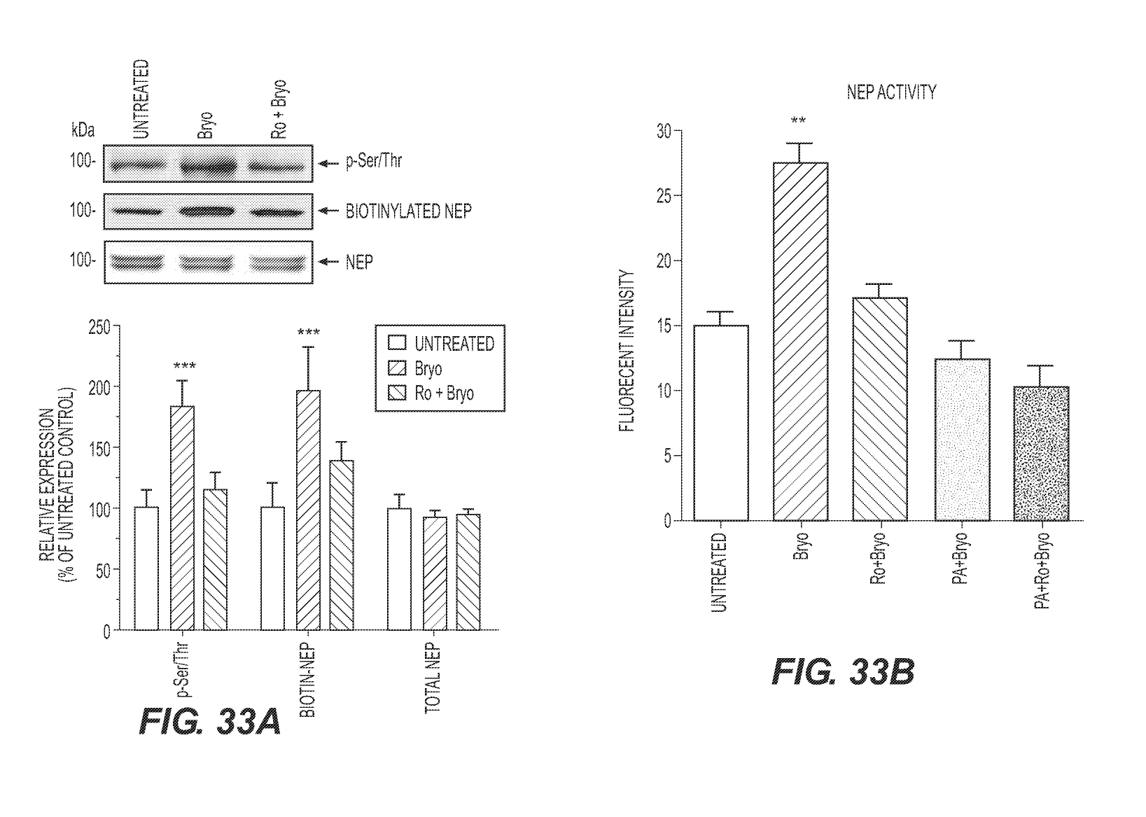

Protocol: After SH+hNEP cells were untreated or treated with bryostatin (Bryo, 1 nM), or pre-treated with PKC inhibitor Ro 32-0432 (Ro, 2 μM) for 30 min and then treated with Bryo for 1 hour, cell surface located proteins were biotinylated and pulled down using streptavidin beads, followed by immunoprecipitation using a neprilysin antibody. Immunoprecipitates were subjected to Western blot analysis using phospho-Ser/Thr or neprilysin antibody (Mean+SEM of three independent experiments, **P<0.01, Bryo compared with untreated; #P<0.01, Ro+Bryo compared with Bryo). Intact SH+hNEP cells were incubated in the absence or presence of bryostatin (Bryo, 1 μM) for 1 hr. Some cells were pre-treated with PKC inhibitor Ro 32-0432 (Ro, 2 μM) for 30 min before Bryo treatment. Cells were then lysed and neprilysin activity was measured. 50 μg of total lysates were separately incubated with 0.5 mM glutaryl-Ala-Ala-Phe-4-methoxy-2-naphthylamide as a substrate. Further incubation with leucine aminopeptidases released free 4-methoxy-2-naphthylamide that was measured fluorometrically at an emission of 425 nm. For the inhibition study cells were pre-incubated with phosphoramidon (PA, 10 μM) for 5 min before the addition of the substrate (Mean+SEM of three independent experiments, **P<0.01, Bryo compared with untreated; #P<0.01, Ro, PA, or PA+Ro compared with Bryo). Results are shown below in FIG. 33 (A—Western Blot results; B—fluorometric results).

ECE Activation

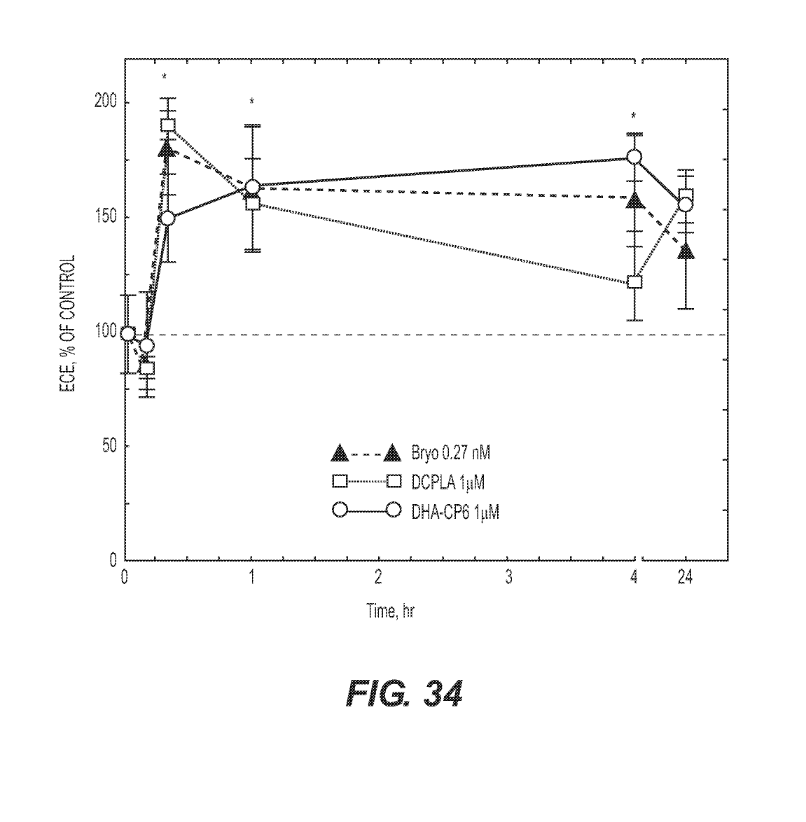

PKCε overexpression has been reported to activate endothelin converting enzyme (ECE). The effect of PKC activators on ECE was measured here with Bryostatin, DCP-LA, and DHA-CP6. All produced a sustained increase in ECE activity. Since ECE does not possess a diacylglycerol-binding C1 domain or a PKC-like phosphatidylserine-binding C2 domain, this suggests that the activation was not due to direct activation of ECE, but must have resulted from indirect activation of ECE or some ECE-activating intermediate by PKC. Protocol: Bryostatin (0.27 nM), DCP-LA (1 μM), DHACP6 (1 μM), EPA-CP5 (1 μM), AA-CP4 (1 μM), or ethanol alone were added to SH-SYSY cells growing on 12- or 24-well plates. After various periods of time, the cells were collected and ECE activity was measured fluorometrically as showing in FIG. 34 below (* p<0.05, ** p<0.001).

Inhibition of GSK3β-Phosphorylation of Tau

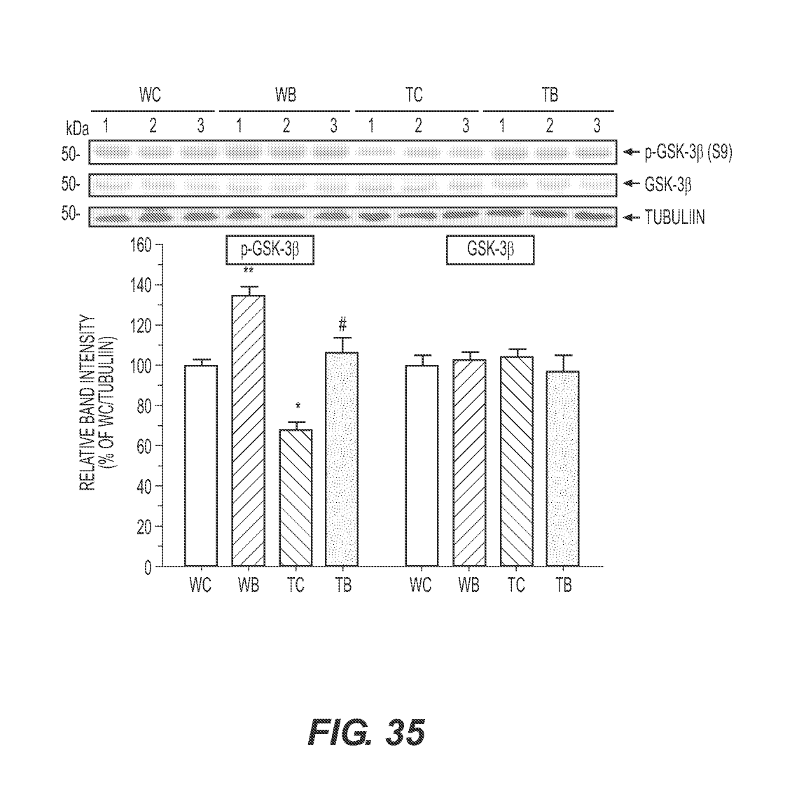

Protocol: Hippocampal tissue from wild type control mice with vehicle (WC), wild type mice with bryostatin-1 (WB, 20 μg/m2, i.v., 2 doses/wk for 13 wk), fragile X mice with vehicle (TC), and fragile X mice with bryostatin-1 (TB) were dissected and total GSK-3β protein was extracted and used for Western Blot analysis using GSK-3β and phospho-GSK-3β (Ser9) antibodies. A representative gel image is shown in FIG. 35 below from three independent experiments and all data are presented as % (Mean+SEM; **P<0.01, WB compared with WC; *P<0.05, TC compared with WC; #P<0.01, TB compare with TC).

Activation of α-Secretase

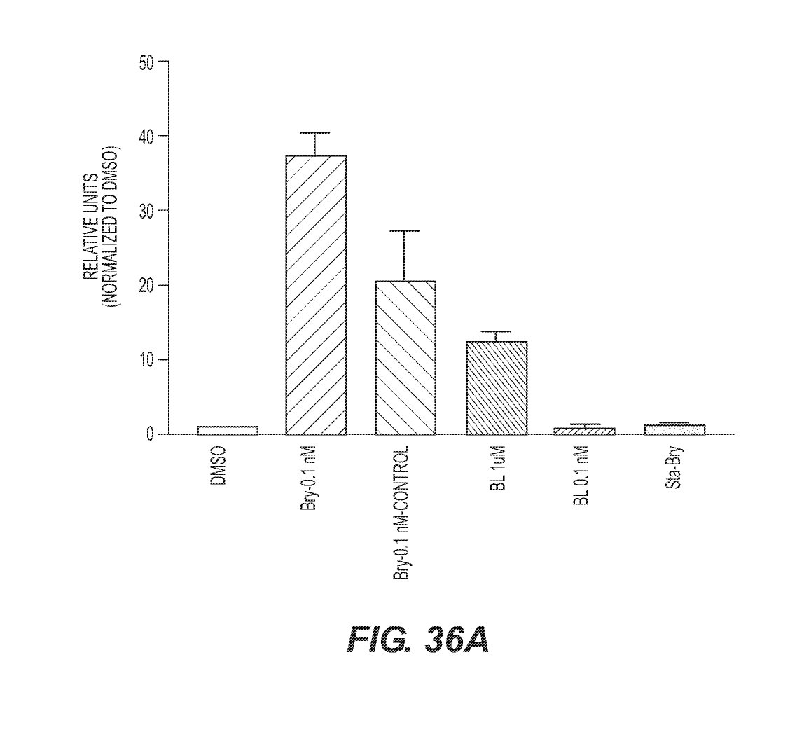

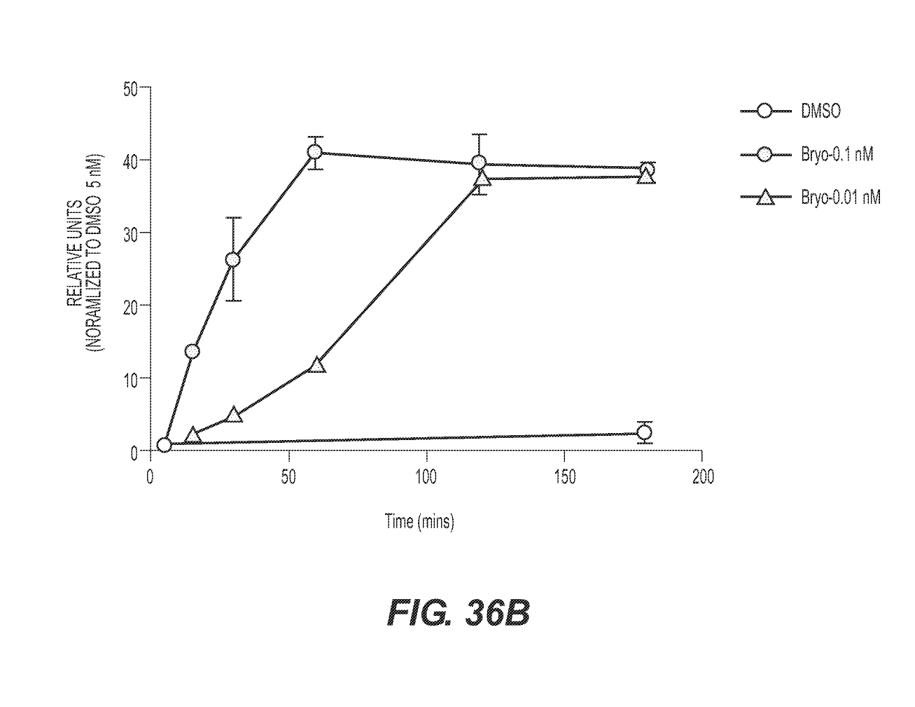

Protocol: An Alzheimer's disease cell line was incubated with bryostatin (0.1 nM), Benzolactam (0.1 nM or 1.0 μM), DMSO, pre-treated with staurosporin (100 nM) plus bryostatin (0.1 nM) for three hours. The amount of sAPP-α in the medium was measured with the results shown below in FIG. 36. The results in A demonstrate that bryostatin (Bry, 0.1 nM, solid bar) dramatically enhanced the amount of sAPP-α in the medium after 3 h of incubation in a well characterized autopsy confirmed AD cell line (P<0.0001, ANOVA). The graph units are relative to the vehicle, DMSO, alone (1). Bryostatin was significantly (P<0.001, Tukey's posttest) more potent than another PKC activator, BL, at the same concentration (0.1 nM). Pretreatment (rightmost bar) with staurosporin (Sta, 100 nM) completely abolished the effect of bryostatin (0.1 nM). Bryostatin was also effective in enhancing secretion in two control cell lines, although to a lesser extent than in the AD cell line (hatched bar). A time course (for the AD cell line) is depicted in B in FIG. 36. The secretion is clearly near enhanced by 15 min of incubation (bryostatin (Bryo), 0.1 nM) and near maximal at 160 min of incubation, remaining elevated up to 3 hours. Bryostatin at a lower concentration, 0.01 nM, was much slower but had about the same effect on secretion after 120 min of incubation.

What is claimed is:

1. A method of identifying a neuroprotective PKC activator comprising:
analyzing a compound to determine whether the compound comprises the following attributes: (a) non-tumorigeneic, (b) non-toxic, (c) brain accessible, (d) α and ε specificity or ε specificity, with ±30% delta activity, (e) results in minimal down regulation of the ε-isozyme, (f) synaptogenic, and (g) anti-apoptoic, wherein when the compound comprises attributes (a) through (g), the compound is a neuroprotective PKC activator.

2. The method of claim 1, further comprising analyzing the compound to determine whether the compound is protective against ASPD.

3. The method of claim 1 or 2, further comprising analyzing the compound to determine whether the compound is protective against in vivo neurodegeneration.

4. The method of any one of claims 1-3, further comprising analyzing the compound to determine whether the compound enhances learning and memory in a normal animal model.

5. The method of any one of claims 1-4, further comprising analyzing the compound to determine whether the compound induces downstream synaptogenic biochemical events.

6. The method of any one of claims 1-5, further comprising analyzing the compound to determine whether the compound increases of Activity of A-β degrading enzymes.

7. The method of any one of claims 1-6, further comprising analyzing the compound to determine whether the compound inhibits GSK3β, wherein when the compound comprises at least 5 of the attributes the compound is a neuroprotective PKC activator.

8. The method of any one of claims 1-7, further comprising analyzing the compound to determine whether it activates alpha-secretase.
